(19) United States
(12) Patent Application Publication
(10) Pub. No.: US 2024/0148771 A1
(43) Pub. Date: May 9, 2024

(54) COMPOSITIONS AND METHODS FOR MODIFYING A PREDETERMINED TARGET NUCLEIC ACID SEQUENCE

(71) Applicant: TARGETGENE BIOTECHNOLOGIES LTD., D.N. HEFER (IL)

(72) Inventors: Yoel Moshe SHIBOLETH, D.N. HEFER (IL); Dan Michael WEINTHAL, Be'er Sheva (IL)

(73) Assignee: TARGETGENE BIOTECHNOLOGIES LTD., KIBBUTZ MAGAL (IL)

(21) Appl. No.: 18/317,447

(22) Filed: May 15, 2023

Related U.S. Application Data
(63) Continuation of application No. 15/449,468, filed on Mar. 3, 2017, now Pat. No. 11,690,866.
Application No. 15/449,468 is a continuation of application No. 14/364,216, filed on Jun. 10, 2014, now Pat. No. 11,458,157.
(60) Provisional application No. 61/576,423, filed on Dec. 16, 2011.

(51) Int. Cl. A61K 31/7105; A61K 31/711; A61K 31/7115; A61K 31/712; A61K 31/7125; A61K 31/713; A61K 38/00; C07K 19/00; C12N 9/22; C12N 15/00; C12N 15/10; C12N 15/62; C12N 15/82; C12N 15/90

(52) U.S. Cl. CPC A61K 31/7105; A61K 31/711; A61K 31/7115; A61K 31/712; A61K 31/7125; A61K 31/713; A61K 38/00; C07K 19/00; C12N 9/22; C12N 15/00; C12N 15/102; C12N 15/62; C12N 15/8213; C12N 15/825; C12N 15/8251; C12N 15/902; C12N 15/907; A61K 48/00; C07K 2319/01

(57) ABSTRACT

Provided herein are compositions and methods for modifying a predetermined nucleic acid sequence. A programmable nucleoprotein molecular complex containing a polypeptide moiety and a specificity conferring nucleic acid (SCNA) which assembles in-vivo, in a target cell, and is capable of interacting with the predetermined target nucleic acid sequence is provided. The programmable nucleoprotein molecular complex is capable of specifically modifying and/or editing a target site within the target nucleic acid sequence and/or modifying the function of the target nucleic acid sequence.

### CROSS-REFERENCE TO RELATED APPLICATIONS

This application is a Continuation of U.S. patent application Ser. No. 15/449,468, filed Mar. 3, 2017; which is a Continuation of U.S. patent application Ser. No. 14/364,216, filed Jun. 10, 2014, now U.S. Pat. No. 11,458,157, issued on Oct. 4, 2022; which is a U.S. National Stage of international Application No. PCT/IL2012/050528, filed Dec. 16, 2012; which claims benefit of U.S. Provisional Patent Application No. 61/576,423, filed Dec. 16, 2011. All the foregoing applications are incorporated by reference in their entirety herein.

### INCORPORATION OF SEQUENCE LISTINGS

A sequence listing conforming to the rules of WIPO Standard ST.26 is hereby incorporated by reference in its entirety. Said sequence listing has been filed as an electronic document via PatentCenter encoded as XML in UTF-8 text. The electronic document is named “P34507US05_SL.xml”, and is 353,931 bytes in size (measured in MS-Windows®) and created on Jul. 6, 2023.

### FIELD OF THE INVENTION

The present invention relates to compositions and methods for targeting and modifying nucleic acid sequences utilizing a programmable molecular complex.

### BACKGROUND OF THE INVENTION

A major area of interest in biology and medicine is targeted alteration of genomic nucleotide sequences. Such alterations include insertion, deletion and replacement of endogenous chromosomal nucleic acid sequences. Past attempts have been made by others to alter genomic sequences by different techniques.

Gene targeting is a biotechnological tool desired for genome manipulation or genome functional modification. Gene targeting can induce a change in a specific genomic location which may or may not, be related to coding sequences.

In a gene targeting event, a predefined endogenous gene, or another predefined endogenous nucleic acid sequence, is either targeted for cleavage resulting in deletion, mutation, insertion or replacement or targeted for chemical modification by targeted gene-functional modification. One advantage of gene-targeting over untargeted transgenic organism production is the possibility to modify or delete existing genomic sequences without insertion of foreign DNA, or alternatively, place a foreign donor DNA, by insertion or replacement, in a predefined locus. It is advantageous to be able to thus manipulate a sequence without superfluous sequences, as these are undesired by breeders, farmers, consumers and regulatory agencies, and while many techniques for avoiding such sequences have been suggested, each suffers from its own shortcomings.

The strategies for gene targeting in Eukaryotes are dependent on two cellular dsDNA break repair mechanisms: The homologous recombination (HR) and non-homologous-end-joining (NHEJ) repair pathways. In NHEJ gene insertions depend on the existence of a dsDNA break which may occur randomly (e.g. through radiation or oxidative damage) or be directed by a nuclease such as a TALE nuclease (TALEN), meganuclease or a zinc-finger nuclease (ZFN). HR can be induced by dsDNA breaks. In HR, a dsDNA break is not essential, but may improve the efficiency if located near the recombination site.

Extensive research has been conducted on HR mediated gene targeting which functions usefully well in many organisms such as bacteria, yeast and the primitive plant, moss. HR has also been utilized in higher organisms such as drosophila, mice, and humans. Rates of HR in these organisms are about 10\hat{ }−6, and can be increased to over 10\hat{ }−2, in assisted HR, by creating a gene specific DSB. Low rates of transformants are one reason these methods are not prevalent in gene therapy or breeding programs.

Various techniques for modifying nucleic acids in-vivo have been suggested and can be divided into enzyme based or nucleotide based methods. In general, enzyme based methods use a DNA-binding protein which has both a desired catalytic activity and the ability to bind the desired target sequence through a protein-nucleic-acid interaction in a manner similar to restriction enzymes. Examples include meganucleases which are naturally occurring or engineered rare sequence cutting enzymes, zinc finger nucleases (ZFNs) or transcription activator-like nucleases (TALENs) which contain the FokI catalytic nuclease subunit linked to a modified DNA binding domain and can cut one predetermined sequence each. In ZFNs the binding domain is comprised of chains of amino-acids folding into customized zinc finger domains. In TALENs, similarly, 34 amino acid repeats originating from transcription factors fold into a huge DNA-binding domain. In the event of gene targeting, these enzymes can cleave genomic DNA to form a double strand break (DSB) or create a nick which can be repaired by one of two repair pathways, non-homologous end joining (NHEJ) or homologous recombination (HR). The NHEJ pathway can potentially result in specific mutations, deletions, insertions or replacement events. The HR pathway results in replacement of the targeted sequence by a supplied donor sequence. One disadvantage of these protein-only based methods is the long and laborious necessity to design and supply a different protein for every desired target sequence. Other disadvantages include the somewhat limited subset of nucleic acid triplets or sequences recognized by ZFNs and meganucleases respectively. Moreover, even a six-Zinc-finger ZFN, which is very difficult to construct, is limited to a binding site of only 18 nucleotides, and as 18 nucleotides are statistically not sufficient to confer sequence specificity in the sequence space, or complexity, of a whole genome these must be supplied as heterodimers. Moreover, the nature of ZFNs and TALENs requires functionality screening and even successful nucleases may show poor gene-targeting efficiency.

For nucleotide based methods, nucleic acids are supplied to the organism and endogenous processes bring about DNA repair or gene-targeting through unassisted homologous recombination or integration of the oligonucleotide into the genome. These nucleic acids can be supplied using viral-vectors, plasmid vectors, T-DNA vectors and double-stranded DNA oligonucleotides. Shorter nucleotides termed Triple-helix forming oligonucleotides (TFOs) are used for Oligonucleotide-based mismatch repair, and can attain repair of point mutations or up to 4 nucleotide repair. There is ample evidence that these methods too are dependent on the formation of DSBs which can be random, randomly induced or locally induced by enzymatic or chemical modifications through enzymes or reactive chemicals covalently bound to the supplied nucleic acid. Double strand breaks (DSB) in DNA are necessary for HR. Specific pre-existing DSBs are not essential but improve efficiency. Natural breaks in DNA are randomly located and rare, and thus efficiency, thus, must be low (10\hat{ }−6). DSBs can be randomly induced by ionizing radiation or oxidizing chemicals, improving efficiency at the expense of genotoxicity. In an improvement to this system, assisted HR or repair has been performed in the past using non-enzymatic DNA cleavage assisted by chemical modification of the terminus of a nucleic acid. These modifications include EDTA-Fe or photoactivatable Psoralen and were used for the production of a sequence specific DSB in dsDNA when incorporated in vitro to form a triple helix. An additional method uses oligonucleotides, or modified oligonucleotides, derived from single-stranded DNA (ssDNA), otherwise known as “small synthetic single-stranded oligodeoxynucleotides (ODNs or ssODNs). However, while oligonucleotide based methods may result in relatively efficient point mutations in mammalian cell genomes, these are restricted to this mode of editing.

Oligonucleotide-enzyme conjugates are a combination of the two methods comprising of a nucleic acid covalently bound in-vitro to a catalytic enzyme prior to supplying the conjugate to the organism. These methods, in contrast to enzyme-only methods are modular, allowing preparation of conjugates aimed at a diversity of target sequences. The main disadvantage of oligonucleotide-enzyme conjugates is that they cannot self-assemble in vivo, thereby severely limiting their usefulness for genome editing in vivo. Additional critical disadvantage of such systems known in the art is that in uses of these conjugates the enzyme component is active as a monomer, and thus any binding of the enzyme to a nucleic acid, specific or not, will result in cleavage. Such non-specific cleavage severely reduces the safety of such systems, as they might introduce undesired changes/mutations at undesired locations.

Non-conjugated oligonucleotide-protein systems have also been used to cleave a ssDNA substrate. In this system a Class-IIS Restriction Enzyme, FokI, which cleaves outside its recognition site was used in vitro, in conjunction with a hairpin forming oligonucleotide which reconstitutes the FokI recognition sequence, with a PolIk enzyme and dNTPs to create a double-strand section of DNA primed by the oligonucleotide to be cleaved. In this system, not only the intended sequence is cleaved, but any naturally occurring FokI site will be recognized and the sequence adjacent to it will be cleaved. As FokI has only a 5-nucleotide recognition site this implies there are thousands of potential cleavage sites in a whole genome, rendering this system useless for genome editing.

In higher plants and humans, in contrast to other organisms where HR can be used for gene-targeting, the NHEJ pathway is the predominant endogenous mechanism. The plant DNA-repair machinery does not permit efficient HR between donor and chromosomal DNA. Indeed, it is widely accepted that foreign donor DNA molecules, which are often delivered by Agrobacterium-mediated genetic transformation, are recognized by the plant Non-Homologous End Joining (NHEJ) pathway, which leads to their random integration throughout the host genome. Most current plant transformation methods, thus, are not considered gene targeting, as in these methods, sequences are randomly inserted in the genome, and as an undesirable side effect, may disrupt an existing gene, and are often inserted in multiple copies, or contain undesired plasmid, marker or bacterial sequence remnants.

Methods for induction of specific dsDNA breaks, useful for assisted HR and directed NHEJ, utilize expression of nucleases in vivo. These include rare-sequence cutting nucleases (rare-cutters) such as meganucleases or chimeric meganucleases, derived from homing endonucleases, custom-made recombinant Zinc-Finger-Nucleases (ZFNs), or custom-made recombinant TAL effector nucleases. In these methods, recognition of the cleaved target site, is achieved by the interaction of a protein domain or subunit which naturally recognizes a specific nucleotide sequence, or is engineered specifically to recognize a specific nucleotide sequence and is not based on polynucleotide-polynucleotide hybridization or base-pairing. For example, Zinc Finger Nucleases are chimeric proteins, constructed as hybrids between the FokI nuclease subunit and synthetic zinc-finger (ZF) domains. Zinc Finger Nucleases do not contain a nucleic acid component. ZFNs are designed to specifically recognize nucleotide triplets through a combination of several ZF motifs. ZFNs cannot be constructed to recognize all sequences due to their inherent ability to recognize only a limited subset of nucleotide triplets. Use of ZFN heterodimers, whereby two different ZFNs, which are inactive as a monomer are delivered concomitantly, has a positive effect on specificity, though this complicates the design further and reduces the choice of target sequences. ZFNs have also been utilized to create artificial transcription factors both for activation and for repression of genes, for altering gene regulation. However, such Zinc finger based transcription factors cannot bind all sequences, being limited in length of recognition site and limited to several specific tri-nucleotide motifs, and thus cannot be utilized to activate or suppress all possible genes.

For example, Schierling et. al., disclose a novel zinc finger nuclease platform with a sequence-specific cleavage module. For example, Eisenschmidt K, et. al. disclose a programmed restriction endonuclease for highly specific DNA cleavage. For example, WO 2006/027099 is directed to enzyme conjugates with a programmable specificity, which react in a highly specific manner with DNA.

Kubo et. al., for example, disclose the control of intracellular delivery of oligonucleotides by signal peptides and genetic expression in human cells. Jinek et. al., disclose a programmable Dual-RNA-Guided DNA endonuclease in adaptive bacterial immunity.

WO 2012/129373, for example, is directed to methods for producing a complex transgenic trait locus.

Nevertheless, there is still an unmet need in the art for safe, reliable, modular, and inexpensive compositions and methods that allow the specific targeting and modifying of target nucleic acid sequences in-vivo.

### SUMMARY OF THE INVENTION

The present invention provides compositions and methods for targeting and modifying nucleic acid sequences, in-vivo or in-vitro. According to some embodiments the novel composite programmable molecular complex (nucleo-protein complex) provided herein is used to edit or functionally modify a predetermined nucleic acid sequence target precisely, reliably and cost effectively.

In some embodiments, the molecular complex disclosed herein is used for gene-targeting and/or targeted gene-functional modification including, but not limited to, generation of breaks in one or two strands of the target nucleic acid to initiate gene mutation, deletion, gene replacement, and integration of a foreign nucleic acid molecule, or for its chemical, conformational, or biological functional modification.

According to some embodiments, the molecular complex disclosed herein comprises a) a chimeric polypeptide (that may be encoded by a polynucleotide molecule), the chimeric polypeptide comprising: (i) a functional (effector) domain (FD) capable of modifying a target site; and (ii) a linking domain (LD); and (b) a specificity conferring nucleic acid (SCNA), the SCNA comprising: (i) a nucleotide sequence complementary to a region of a target nucleic acid flanking the target site; and (ii) a recognition region capable of specifically attaching to the linking domain of the polypeptide; whereby assembly of the polypeptide and the SCNA within a host/target cell forms a functional, programmable, nucleoprotein molecular complex, capable of specifically modifying the target nucleic acid at the target site.

In some embodiments, the present invention provides an advantageous composition comprising a protein effector module (or a nucleic acid molecule encoding the same) and a programming/targeting nucleic-acid module which can self-assemble in-vivo into a specific, active nucleic acid modifying molecular nucleoprotein complex. In this complex, the nucleic acid, also termed herein as a “programming moiety”, “programming oligonucleotide” or “specificity-conferring nucleic acid” (SCNA) provides the specificity and binding capabilities of the molecular complex to the target nucleic acid through base-pairing of said specificity-conferring nucleic acid and a target nucleic acid. The protein effector component or module of this complex is designed to bind/link/attach to the specificity determining nucleic acid by a chemical moiety attached to the oligonucleotide, a modification of a nucleotide or nucleotides on the oligonucleotide, a specific recognition sequence on the oligonucleotide, and the like, or combinations thereof. Advantageously, the compositions and methods disclosed herein confer higher specificity with a wide range of desired target sequences, are less genotoxic, modular in their assembly, reliable, utilize a single platform without customization, practical for independent use outside of specialized core-facilities, and have a shorter development time frame and reduced costs.

The activity of the protein module may result in the modification of the target nucleic acid sequence and/or the functional modification of the target nucleic acid. Target nucleic acid modification may include, but is not limited to: mutation, deletion, insertion, replacement, binding, digestion, nicking, methylation, acetylation, ligation, recombination, helix unwinding, chemical modification, labeling, activation, and inactivation or any combinations thereof. Target nucleic acid functional modification may lead to, but is not limited to: changes in transcriptional activation, transcriptional inactivation, alternative splicing, chromatin rearrangement, pathogen inactivation, virus inactivation, change in cellular localization, compartmentalization of nucleic acid, and the like, or combinations thereof. Any editing action or other modification effected by the protein moiety is directed or guided to an intended (predefined) specific target nucleic acid by its linkage to the specificity-conferring nucleic acid. Advantageously, use of each single type of protein component may be combined with an unlimited assortment of nucleotide-sequences of specificity determining nucleic acids concomitantly or separately, to allow similar action on different sections of intended target nucleic acid. This allows overcoming shortcoming of state of the art methods, by providing a versatile, reliable and cost effective methods and compositions for modifying predetermined nucleic acid sequence targets. Thus, if used in one receptacle or organism, only one type of protein is to be provided with any combination or multiplicity of specificity determining nucleic acid types. This also includes the possibility to concomitantly use more than one type of protein component with more than one type of specificity determining nucleic acids.

According to some embodiments, the complex disclosed herein is modular and can self-assemble within a target cell either in vivo or in vitro, allowing the supply of one type of protein moiety at a time with one or a multiplicity of specificity-determining oligonucleotides concomitantly. Furthermore, in some embodiments, the protein component can be delivered to a desired cell(s) and expressed in vivo, awaiting the delivery of any appropriate SCNA at a later time. In some embodiments, the protein component and the SCNA may be delivered simultaneously, or essentially simultaneously. Thus, the combination of the protein component and the SCNA, preferably within the desired target cell, may accomplish the induction of specific genomic double strand breaks (DSBs), or any other desired nucleic acid modification, in vivo. The methods of the present invention are not limited to the introduction of point mutations to the target nucleic acid, as the molecular complex can target any nucleic-acid sequence or pair of sequences, cut/restrict/cleave in close proximity to them, and consequentially delete a small or large nucleic acid section, or cut/restrict/cleave the sequence in order to initiate a removal, or an insertion, or a replacement of any nucleic acid sequence.

Advantageously, the present invention, in embodiments thereof, discloses for the first time expression of a protein component in-vivo and its binding/attachment to the SCNA(s) by self-assembling in-vivo to form a molecular complex in-vivo, without the need of prior covalent/chemical linking between the protein moiety and the targeting nucleic acid. According to embodiments of the present invention, in contrast to the oligonucleotide-based systems known in the art, the SCNA bound to the protein is not intended to function as a donor, but rather as a specificity conferring moiety, and does not become part of the modified nucleic acid. Furthermore, in some embodiments of the present invention, the SCNA can be expressed in vivo in a manner that causes the assembly of all the components of the molecular complex with a single delivery event. Furthermore, according to some embodiments, the effector protein can be designed to be active only upon it's dimerization (i.e. it must form a dimer to be active), whereby the dimerization can be controlled such that an active dimer can only form when it is targeted/programmed by an SCNA and bound to its target site, for example, when the molecular distances between the monomeric partners (proteins) of the dimer are precise enough. Thus, advantageously, the molecular complex is activated only at its intended target site, thereby enhancing specificity and reliability. According to further embodiments, one protein component may be expressed to form/produce homo-dimers, each programmed/targeted by a different oligonucleotide conferring specificity. Additionally, as viral expression systems, which are known in the art for use for protein expression in-vivo, are often limited to the production of one protein due to size constraints, and are often exclusive to similar viruses due to cross protection, using one protein component has thus a critical advantage for that mode of delivery. Furthermore, in contrast to other methods known in the art (such as ZFNs and meganucleases), which have a limited subset of recognition sequences, the programming oligonucleotides (SCNAs) disclosed herein, have an infinite repertoire of sequences, thus conceivably achieving extreme sequence specificity in high complexity genomes. Moreover, as many programming oligonucleotides can be supplied concomitantly with a single protein effector moiety, it is possible to modify more than one target at the same time, providing additional advantages over methods know in the art. This can be useful, for example, for rapidly knocking out a multiplicity of genes, or for inserting several different traits in different locations, or for tagging several different locations with one donor nucleotide tag.

According to some embodiments, since an unprogrammed protein component (i.e. a protein not attached/linked to a programming oligonucleotide) has no or very low affinity to target nucleic acids, improved specificity and safety and reduced genotoxicity are advantageously obtained. As detailed above, the effector or catalytic domain of the protein component is only active upon dimerization, whereby at least two programming oligonucleotides (SCNAs) must bind the target flanking sequences to cause protein dimerization and activation. Two sufficiently long programming oligonucleotides can impart the very high theoretical specificity needed in high complexity genomes by creating extensive complementarity with the binding sites. Since the unprogrammed expressed protein has no affinity to the target nucleic acid it does not bind, and/or modify the target nucleic acid. Thus, in applications where, for example, the programming oligonucleotides are delivered/supplied separately to the target cell (which already expresses the unprogrammed protein component), or in conditions where oligonucleotides are depleted from the target cell (for example, by dilution or degradation) no unspecific cleavage can occur, thereby increasing safety and reducing genotoxicity.

Thus, according to embodiments of the present invention, both directed non-homologous-end-joining (NHEJ) and assisted homologous recombination (HR) may be utilized specifically and in a programmable manner to achieve one or more of the following:
1) Mutate a DNA sequence by cleaving inside it, creating a double strand break (DSB), to be somewhat degraded by the endogenous nucleases and re-ligated by the endogenous NHEJ DNA repair mechanism to create either an in-frame deletion and/or a frame-shift mutation of the DNA. As opposed to T-DNA or transposon insertion lines in plants, this method of deletion or mutation of an endogenous gene leaves behind no foreign DNA and the plant might be termed non-transgenic by some definitions. In NHEJ one or more nucleotides may also be added in the DSB in a yet uncharacterized endogenous mechanism, essentially achieving the same effect of frame shifting or mutation.
2) Delete a stretch of DNA sequence by cleaving two sequences flanking it, to be re-ligated by the endogenous NHEJ DNA repair mechanism, or by assisted HR by cleaving in or near the sequence to be deleted and supplying a donor DNA which is subsequently recombined into the target, and which contains sequences flanking the sequence to be deleted in the target.
3) Insert a donor nucleic acid into a DSB by cleaving a target nucleic acid and supplying a Donor DNA to be either ligated directly into the gap by the NHEJ mechanism, or preferably, supplying a donor that has homology to the ends of the gap to be recombined and ligated into the gap by assisted HR.
4) Replace a target nucleic acid sequence by cleaving both sequences flanking it, and supplying a donor nucleic acid to be inserted, to be ligated within the target flanking sequence either by NHEJ, or preferably, recombined and ligated by HR, by adding sequences similar to the target nucleic acid, or those flanking it, at the termini of the donor.

According to some embodiments, and without wishing to be bound to theory or mechanisms, the advantages of the compositions and methods disclosed herein, include the creation of a general enzymatic complex construction scheme that can target an unlimited selection of sequences. Once a protein component has been optimized for a specific purpose (e.g. dsDNA cleavage), this same protein can be used with an unlimited selection of programming nucleic acid (SCNA) sequences. Thus, the diversity of target sequences to be affected is achieved by the design of the SCNA, without the difficult and time-consuming necessity of protein re-design and optimization, which is inherent in other methods known in the art, such as, TALENs, ZFNs and Meganucleases, where the protein itself must be changed and adapted for every target sequence. Designing and preparing synthetic SCNAs is relatively simple, rapid and relatively inexpensive. It is also possible, in some embodiments of this invention, to produce SCNAs in-vivo, circumventing the necessity to deliver chemically synthesized SCNAs to a cell. Furthermore, SCNAs can be designed to base pair to almost any desired target sequence, and thus, can direct the molecular complex to almost any target sequence. Moreover, several target sequences may be used in the same cell concomitantly. For example, in editing functions which require more than one cleavage site, such as deletion or replacement of specific stretches of nucleic acid, by simply providing four different SCNAs and one protein moiety.

According to some embodiments, there is thus provided a nucleo-protein composition for modifying a predetermined target site in a target nucleic acid sequence in a target cell, the composition comprising: a polynucleotide molecule encoding a polypeptide, or a polypeptide, said polypeptide comprising: (i) a functional (effector) domain (FD) capable of modifying said target site, the functional domain being devoid of a specific nucleic acid binding site; and (ii) a linking domain (LD), capable of interacting with a specificity conferring nucleic acid (SCNA), wherein the linking domain being devoid of a specific target nucleic acid binding site; and; (b) the specificity conferring nucleic acid (SCNA) or a nucleic acid encoding for the SCNA, the SCNA comprising: (i) a nucleotide sequence complementary to a region of the target nucleic acid flanking the target site; and (ii) a recognition region capable of specifically attaching to the linking domain of the polypeptide with high binding affinity; whereby assembly of the polypeptide and the SCNA within the target cell forms a functional nucleoprotein complex, capable of specifically modifying said target nucleic acid at the target site.

In some embodiments, the functional domain comprises a catalytic domain. In some embodiments, the polypeptide further comprises a subcellular localization domain.

In some embodiments, modifying the target nucleic acid is selected from: mutation, deletion, insertion, replacement, binding, digestion, double-strand-break creation, nicking, methylation, acetylation, ligation, recombination, helix unwinding, chemical modification, labelling, activation and inactivation.

According to some embodiments, the SCNA comprises a nucleic acid molecule selected from the group consisting of a single-strand DNA, a single strand RNA, a double strand RNA, a modified DNA, a modified RNA, a locked-nucleic acid (LNA) and a peptide-nucleic acid (PNA) or combinations thereof.

In some embodiments, the recognition region of the SCNA comprises a modification selected from 5′-end modification, 3′-end modification, and internal modification. In some embodiments, the chemical modification is selected from the group consisting of a nucleotide modification, and addition of a non nucleotide moiety. In some embodiments, the the non nucleotide moiety is selected from: Biotin, Fluorescein, Amine-linkers, oligo-peptides, Aminoallyl, a dye molecule, fluorophores, Digoxigenin, Acrydite, Adenylation, Azide, NHS-Ester, Cholesteryl-TEG, Alkynes, Photocleavable Biotin, Thiol, Dithiol. In some embodiments, the nucleotide modification is selected from the group consisting of phosphate, 2-Aminopurine, Trimer-20, 2,6-Diaminopurine, 5-Bromo-deoxiUridine, DeoxiUridine, Inverted dT, dideoxi-nucleotides, 5-methyl deoxyCytidine, deoxyInosine, 5-nitroindole, 2-O-methyl RNA bases, Iso-dC, Iso-dG, Fluorine modified bases and Phosphorothioate bonds. In some embodiments, the modification is selected from the group consisting of a nucleotide modification, Biotin, Fluorescein, Amine-linkers, oligo-peptides, Aminoallyl, a dye molecule, fluorophores, Digoxygenin, Acrydite, Adenylation, Azide, NHS-Ester, Cholesteryl-TEG, Alkynes, Photocleavable Biotin, Thiol, Dithiol, Modified bases, phosphate, 2-Aminopurine, Trimer-20, 2,6-Diaminopurine, 5-Bromo-deoxiUridine, DeoxiUridine, Inverted dT, dideoxi-nucleotides, 5-methyl deoxyCytidine, deoxyInosine, 5-nitroindole, 2-O-methyl RNA bases, Iso-dC, Iso-dG, Flourine modified bases and Phosphorothioate bonds, and proteins covalently bound by their interaction with the specific nucleotide sequences. In some embodiments, proteins covalently bound by their interaction with the specific nucleotide sequences may be selected from, but not limited to: Agrobacterium VirD2 protein, Picornavirus VPg, Topoisomerase, PhiX174 phage A protein, PhiX A* protein and any variants thereof.

In some embodiments, the attachment/binding/association between the modification on the SCNA and the linking domain results from an interaction of a binding-pair selected from non-covalent interaction of a binding-pair selected from, but not limited to: Biotin-Avidin; Biotin-Streptavidin; Biotin-modified forms of Avidin; protein-protein; protein-nucleic acid interactions; ligand-receptor interactions; ligand-substrate interactions; antibody-antigen; single chain antibody-antigen; antibody or single chain antibody-hapten; hormone-hormone binding protein; receptor-agonist; receptor-receptor antagonist; IgG-protein A; enzyme-enzyme cofactor; enzyme-enzyme inhibitor; single-strand DNA-VirE2; StickyC—dsDNA; RISC—RNA; viral coat protein-nucleic acid; anti-Fluorescein single-chain variable fragment antibody (anti-FAM ScFV)—Fluorescein; anti-DIG single-chain variable fragment (scFv) immunoglobin (DIG-ScFv)—Digoxigenin (DIG) and Agrobacterium VirD2—VirD2 binding protein; and any variants thereof.

In some embodiments, the recognition region of the SCNA comprises a nucleotide motif capable of specifically attaching/binding/associating to the linking domain of the chimeric protein. In some embodiments, the attachment/association/binding between the nucleotide motif and the linking domain is selected from, but not limited to: Zinc finger protein—Zinc finger motif, restriction enzyme recognition domain-restriction enzyme recognition sequence; DNA binding domain of transcription factor—DNA motif, repressor-operator; Leucine zipper—promoter; Helix loop helix—E box domain; RNA binding motifs comprising Arginine-Rich Motif domains, αβ protein domains, RNA Recognition Motif (RRM) domains, K-Homology Domains, Double Stranded RNA Binding Motifs, RNA-binding Zinc Fingers, and RNA-Targeting Enzymes—cognate specific RNA sequence; HIV-rev protein—Stem IIB of the HIV rev response element (RRE); Bovine immunodeficiency virus (BIV) Tat main binding domain—loop 1 of the BIV trans-acting response element (TAR) sequence; Phage lambda, phi21, and P22 Nproteins—The boxB loop hairpins in the N-utilization (nut) sites in their respective RNAs.

According to some embodiments, there is provided a method for modifying a predetermined target site within a target nucleic acid sequence by a programmable nucleoprotein molecular complex, the method comprising the steps of: a) delivering a nucleic acid sequence encoding a programmable chimeric protein (polypeptide) or the protein (polypeptide) to a host cell; b) delivering a specificity-conferring nucleic acid (SCNA) molecule, or a nucleic acid encoding for the SCNA to said host cell; c) binding of said chimeric protein to the SCNA, thereby targeting the chimeric protein to the predetermined target nucleic acid sequence within the host cell, to form an active programmed nucleoprotein complex; and d) allowing the modification of the predetermined target site of the target nucleic acid sequence by said active programmed nucleoprotein molecular complex.

In some embodiments, there is provided a method for modifying a predetermined target site within a target nucleic acid sequence by a programmable nucleoprotein molecular complex, the method comprising the steps of:
a. delivering a nucleic acid sequence encoding a programmable chimeric polypeptide to a host cell, said chimeric polypeptide comprising:
   (i) a functional domain capable of modifying said target site, the functional domain being devoid of a specific nucleic acid binding site; and
   (ii) a linking domain that is capable of interacting with a specificity conferring nucleic acid, wherein the linking domain being devoid of a specific target-nucleic acid binding site;
b. delivering a specificity-conferring nucleic acid (SCNA) molecule, or a nucleic acid encoding the SCNA to said host cell, said SCNA molecule comprising:
   (i) a nucleotide sequence complementary to a region of the target nucleic acid flanking the target site; and
   (ii) a recognition region capable of specifically attaching to the linking domain of the polypeptide with high binding affinity;
wherein expression of the polypeptide in the cell harboring the SCNA enables attachment of said chimeric polypeptide to the SCNA, forming an active programmed nucleoprotein complex, thereby targeting the chimeric polypeptide to the predetermined target nucleic acid sequence within the host cell, enabling the modification of the predetermined target site of the target nucleic acid sequence by said active programmed nucleoprotein molecular complex.

In some embodiments, the target nucleic acid is DNA. In some embodiments, the target DNA is genomic DNA. In some embodiments, the target nucleic acid sequence is an extra-chromosomal nucleic acid sequence. In some embodiments, the extra-chromosomal target nucleic acid sequence resides in an organelle selected from the group consisting of mitochondria, chloroplast, amyloplast and chromoplast. In some embodiments, the target nucleic acid sequence is a viral nucleic acid sequence. In some embodiments, the target nucleic acid sequence is a prokaryotic nucleic acid sequence. In some embodiments, the target nucleic acid sequence is a synthetic nucleic acid sequence.

In some embodiments, the modification is selected from mutation, deletion, insertion, replacement, binding, digestion, double-strand-break creation, nicking, methylation, acetylation, ligation, recombination, helix unwinding, chemical modification, labelling, activation and inactivation.

In some embodiments, the chimeric protein (polypeptide) comprises a protein moiety having a nucleic acid modifying activity. In some embodiments, the chimeric protein comprises a protein moiety having a nucleic acid functional modifier, wherein the functional modification is selected from the group consisting of transcriptional activation, transcriptional inactivation, RNA transcript silencing, alternative RNA splicing, chromatin rearrangement, cellular parasite and virus inactivation and change in cellular localization or compartmentalization of said target nucleic acid sequence.

In some embodiments, the SCNA comprises a molecule selected from the group consisting of a single-strand DNA, a single strand RNA, a double strand RNA, a modified DNA, a modified RNA, a locked-nucleic acid (LNA) and a peptide-nucleic acid (PNA) or combinations thereof. In some embodiments, the SCNA comprises a specificity-defining sequence configured to specifically interact with the target nucleic acid. The interaction between the SCNA and the target nucleic acid is through base pairing, selected from the group consisting of a full double helix base pairing, a partial double helix base pairing, a full triple helix base pairing, a partial triple helix base pairing, and D-loops or branched forms, formed by said base pairing.

In additional embodiments, the SCNA comprises a recognition region, configured to associate/bind/attach with a linking domain of the chimeric protein. In some embodiments, the recognition region comprises a modification selected from the group consisting of 5′-end modification, 3′-end modification, and internal modification. The modification may be selected from, but not limited to nucleotide modification, Biotin, Fluorescein, Amine-linkers, oligo-peptides, Aminoallyl, a dye molecule, fluorophores, Digoxygenin, Acrydite, Adenylation, Azide, NHS-Ester, Cholesteryl-TEG, Alkynes, Photocleavable Biotin, Thiol, Dithiol, Modified bases, phosphate, 2-Aminopurine, Trimer-20, 2,6-Diaminopurine, 5-Bromo-deoxiUridine, DeoxiUridine, Inverted dT, dideoxi-nucleotides, 5-methyl deoxyCytidine, deoxyInosine, 5-nitroindole, 2-O-methyl RNA bases, Iso-dC, Iso-dG, Flourine modified bases and Phosphorothioate bonds, and proteins covalently bound by their interaction with the specific nucleotide sequences. The proteins covalently bound by their interaction with the specific nucleotide sequences are selected from Agrobacterium VirD2 protein, Picornavirus VPg, Topoisomerase, PhiX174 phage A protein, PhiX A* protein and any variants thereof.

In some embodiments, the association/binding/attachment between the modification on the SCNA and the linking domain results from a non-covalent interaction of a binding-pair selected from: Biotin-Avidin; Biotin-Streptavidin; Biotin-modified forms of Avidin; Protein-protein interactions; protein-nucleic acid interactions; ligand-receptor interactions; ligand-substrate interactions; antibody-antigen interactions; single chain antibody-antigen; antibody or single chain antibody-hapten interactions; hormone-hormone binding protein; receptor-agonist; receptor-receptor antagonist; anti-Fluorescein single-chain variable fragment antibody (anti-FAM ScFV)—Fluorescein; anti-DIG single-chain variable fragment (scFv) immunoglobin (DIG-ScFv)—Digoxigenin (DIG); IgG—protein A; enzyme-enzyme cofactor; enzyme-enzyme inhibitor; single-strand DNA-VirE2; StickyC—dsDNA; RISC—RNA; viral coat protein-nucleic acid and Agrobacterium VirD2—VirD2 binding protein; and any variants thereof.

In some embodiments, binding/association between the specificity conferring nucleic acid sequence and the linking domain of the protein moiety is covalently created in vivo. In some embodiments, the covalent association of the linking domain and the SCNA results from a biological interaction of Agrobacterium VirD2—Right border sequence or any variants thereof, and is created in a bacterium comprising Agrobacterium.

In some embodiments, the recognition region comprises a nucleotide motif capable of interacting/attaching/binding with the linking domain of the chimeric protein. In some embodiments, the interaction pair is selected from: Zinc finger protein—Zinc finger motif; restriction enzyme recognition domain—restriction enzyme recognition sequence; DNA binding domain of transcription factor—DNA motif; repressor—operator; Leucine zipper—promoter; Helix loop helix—E box domain; RNA binding motifs comprising Arginine-Rich Motif domains, αβ protein domains, RNA Recognition Motif (RRM) domains, K-Homology Domains, Double Stranded RNA Binding Motifs, RNA-binding Zinc Fingers, and RNA-Targeting Enzymes—cognate specific RNA sequence; HIV-rev protein—Stem IIB of the HIV rev response element (RRE); Bovine immunodeficiency virus (BIV) Tat main binding domain—loop 1 of the BIV trans-acting response element (TAR) sequence; Phage lambda, phi21, and P22 Nproteins—The boxB loop hairpins in the N-utilization (nut) sites in their respective RNAs.

According to some embodiments, the predetermined target nucleic acid sequence is involved in a genetic trait, and the modification results in changes in the transcription or translation of a genetic element, by a technical procedure selected from the group consisting of permanently replacing, knocking-out, temporarily or permanently enhancing, shutting-off, knocking-down, and frameshifting. In some embodiments, the genetic trait is modified by editing the genetic element sequence itself, its regulatory sequences, genes regulating the gene of interest or their regulatory sequences in a regulatory chain of events.

According to further embodiments, there is provided a nucleo-protein complex, wherein a physical association between the protein moiety and the specificity conferring nucleic acid moiety form a programmed functional complex. In some embodiments, the physical association between the linking domain of the protein moiety and the SCNA is based on an affinity interaction selected from the group consisting of ligand-receptor, ligand-substrate, hydrogen bonds, van der Waals bonds, ionic bonds and hydrophobic interaction.

According to some embodiments, there is provided a host cell having a predetermined genetic modification in a predetermined target site, created by the method disclosed herein. In some embodiments, the host cell may be any type of cell, such as, but not limited to: vertebrate cell, mammalian cell, human cell, animal cell, plant cell, invertebrate cell, nematodal cell, insect cell, and a stem cell.

According to some embodiments, there is provided a transgenic organism or knock out organism, having a predetermined genetic modification formed by the method described herein. In some embodiments, the organism is a plant or an animal.

According to some embodiments, there is provided a method of treating a genetic disease in an organism, the method comprising introducing into a cell of the organism the nucleoprotein programmable molecular complex.

According to some embodiments, there is provided a host cell comprising:
a) a polypeptide comprising: (i) a functional domain capable of modifying a target site in a target nucleic acid sequence in the cell, the functional domain being devoid of a specific nucleic acid binding site; and (ii) a linking domain that is capable of interacting with a specificity conferring nucleic acid and being devoid of a specific target-nucleic acid binding site; and;
(b) a specificity conferring nucleic acid (SCNA) comprising:
   (i) a nucleotide sequence complementary to a region of the target nucleic acid flanking the target site; and (ii) a recognition region capable of specifically attaching to the linking domain of the polypeptide;
whereby assembly of the polypeptide and the SCNA within the host cell forms a functional nucleoprotein complex, capable of specifically modifying the target nucleic acid at the target site.

In some embodiments, there is provided a host cell harbouring: (a) a polynucleotide molecule encoding for a polypeptide, the polypeptide comprising: (i) a functional domain capable of modifying a target site in a target nucleic acid sequence in the cell, the functional domain being devoid of a specific nucleic acid binding site; and (ii) a linking domain that is capable of interacting with a specificity conferring nucleic acid and being devoid of a specific target-nucleic acid binding site; and (b) a specificity conferring nucleic acid (SCNA) comprising: (i) a nucleotide sequence complementary to a region of the target nucleic acid flanking the target site; and (ii) a recognition region capable of specifically attaching to the linking domain of the polypeptide; whereby assembly of the polypeptide and the SCNA within the host cell forms a functional nucleoprotein complex, capable of specifically modifying the target nucleic acid at the target site.

### BRIEF DESCRIPTION OF THE DRAWINGS

FIGS. 1A-B are schematic cartoons showing elements/components of a programmable molecular complex, according to some embodiments comprising a programmable protein moiety (comprising a cellular localization domain, a function domain, an interdomain connector, and a linking domain) and a specificity conferring nucleic acid (SCNA); FIG. 1A depicts a specificity conferring SCNA comprising a modification or label, and FIG. 1B depicts an SCNA without a modification or label;

FIGS. 2A-B are schematic cartoons showing the assembly of the programmable molecular complex, according to some embodiments; FIG. 2A depicts assembly of the programmable molecular complex using the SCNAs of FIG. 1A, and FIG. 2B depicts assembly of the programmable molecular complex using the SCNAs of FIG. 1B;

FIG. 3 demonstrates a 3D Modeled example of a molecular complex designed for cleavage of a predefined nuclear dsDNA target sequence, according to some embodiments;

FIGS. 4A-B are schematic drawings (not to scale) of exemplary mode of assembly of the components of the programmable molecular complex on a target nucleic acid, according to some embodiments; FIG. 4A depicts one mode of assembly of the components of the programmable molecular complex on a target nucleic acid using the SCNAs of FIG. 1B, and FIG. 4B depicts one mode of assembly of the components of the programmable molecular complex on a target nucleic acid using the SCNAs of FIG. 1A;

FIG. 5 is a schematic scheme demonstrating the delivery of the programmable molecular complex to a cell using in-vitro produced SCNAs, according to some embodiments;

FIG. 6 is a general scheme demonstrating the delivery of the programmable molecular complex to a cell using an in-vivo produced SCNA, according to some embodiments;

FIGS. 7A-B are schemes showing non-limiting examples of the delivery of the programming nucleic acid moiety of the molecular complex to a cell using a single-strand DNA SCNA; FIG. 7A depicts a split binary vector encoding a single-strand DNA SCNA in an Agrobacterium cell, and FIG. 7B depicts the cellular import of single-strand DNA SCNAs produced in Agrobacteria and the assembly of the SCNAs with a protein moiety expressed in a cell at a target site, according to some embodiments;

FIGS. 8A-B schematic illustration demonstrating the delivery of the programming moiety of the programmable molecular complex to a cell using RNA SCNAs; FIG. 8A depicts RNA SCNAs derived from an Agrobacterium delivered T-DNA in a cell that expresses a protein moiety and the assembly of the SCNAs with the protein moiety at a target site; and FIG. 8B depicts RNA SCNAs derived from viral genomic RNA in a cell that expresses a protein moiety and the assembly of the SCNAs with the protein moiety at a target site, according to some embodiments;

FIG. 9 shows a schematic illustration (not to scale) of a non-limiting example of a delivery vehicle or vector for concomitant delivery of the composition comprising the components necessary for the assembly of a programmable molecular complex to a susceptible target Eukaryotic cell in a single delivery event, according to some embodiments;

FIG. 10 is a schematic illustration (not to scale) demonstrating the use of a programmed molecular complex to create a mutation in a Target nucleic acid, according to some embodiments.

FIG. 11 is a schematic illustration (not to scale) demonstrating the use of a programmed molecular complex to insert one or a multiplicity of nucleotides into a Target nucleic acid using a supplied Donor nucleic acid, according to some embodiments.

FIG. 12 is a schematic illustration (not to scale) demonstrating the use of a programmed molecular complex to replace one or a multiplicity of nucleotides in a Target nucleic acid using a supplied Donor nucleic acid, according to some embodiments

FIG. 13 is a schematic illustration (not to scale) demonstrating the use of a programmed molecular complex to create a deletion of one or a consecutive multiplicity of nucleotides from a Target nucleic acid, according to some embodiments.

FIG. 14 is a schematic illustration (not to scale) demonstrating the use of a programmed molecular complex to replace one or a multiplicity of nucleotides in a Target nucleic acid using a supplied Donor nucleic acid, according to some embodiments.

FIG. 15 shows a schematic illustration of a non-limiting example of a delivery vehicle or vector (not to scale) for concomitant delivery of the programmable molecular complex protein (PMCP) to a susceptible target Eukaryotic cell together with a target sequence to test its activity, according to some embodiments, and as detailed in Example 10.

FIG. 16 shows a schematic drawing (not to scale) of parameters to empirically determine the optimal distance between SCNA pairs and to test capability of different types of programmed molecular complexes to specifically cleave a target DNA, as detailed in Example 12.

### DETAILED DESCRIPTION OF THE INVENTION

According to some embodiments, there are provided compositions and methods for modifying a predetermined target nucleic acid. Specifically disclosed are methods for modifying a target sequence in-vivo, using a composition which comprises a programmable molecular complex. The programmable molecular complex (also referred to herein as a “nucleo-protein complex”) comprises a protein moiety, (also referred to herein as a “programmable moiety”), and a nucleic acid moiety, (also referred to herein as a “specificity-conferring nucleic acid” (SCNA) or “the programming nucleic acid”). According to some embodiments, the components of the molecular complex self-assemble in-vivo in a living cell, organism, tissue, callus, organ or part thereof, whether differentiated or not, in the presence of a target nucleic acid sequence(s) to form an active, programmed functional molecular complex.

It is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9 and 7.0 are explicitly contemplated.

### Definitions

### About

As used herein, the term “about” refers to +/−10%.

### Administering

“Administering” is directed to providing a pharmaceutical agent or composition to a subject, and includes, but is not limited to, administering by a medical professional and self-administering.

“Parenteral administration,” means administration not through the intestines. Parenteral administration includes, but is not limited to, subcutaneous administration, intravenous administration, or intramuscular administration.

“Subcutaneous administration” means administration just below the skin.

“Intravenous administration” means administration into a vein.

“Intratumoral administration” means administration within a tumor.

“Chemoembolization” means a procedure in which the blood supply to a tumor is blocked surgically or mechanically and chemotherapeutic agents are administered directly into the tumor.

### Antisense

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

### Autonomously Replicating Vectors

“Autonomously replicating vectors” are defined here as to comprise any natural or un-natural nucleic acid sequence capable of replicating within a host, comprising but not limited to viruses, modified viruses, certain recombinant vectors and plasmids, replicons and intracellular parasites.

### Cell

“Cell” is defined here as to comprise any type of cell, prokaryotic or a eukaryotic cell, isolated or not, cultured or not, differentiated or not, and comprising also higher level organizations of cells such as tissues, organs, calli, organisms or parts thereof. Exemplary cells include, but are not limited to: vertebrate cells, mammalian cells, human cells, plant cells, animal cells, invertebrate cells, nematodal cells, insect cells, stem cells, and the like.

### Complement

“Complement” or “complementary” as used herein means Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules. Partial complementary may mean less than 100% complementarity, for example 80% complementarity.

### Delivery Vector

“delivery vector” or “delivery vectors” is directed to any delivery vector which can be used in the present invention to put into cell contact or deliver inside cells or subcellular compartments agents/chemicals and molecules (proteins or nucleic acids) needed in the present invention. It includes, but is not limited to, transducing vectors, liposomal delivery vectors, plasmid delivery vectors, viral delivery vectors, bacterial delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, nucleic acid(s), proteins), or other vectors such as plasmids and T-DNA. These delivery vectors are molecule carriers.

### Dose

“Dose” as used herein means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

### Dosage Unit

“Dosage unit” as used herein means a form in which a pharmaceutical agent is provided. In certain embodiments, a dosage unit is a vial containing lyophilized oligonucleotide. In certain embodiments, a dosage unit is a vial containing reconstituted oligonucleotide.

### Donor Nucleic Acid

“Donor nucleic acid” is defined here as any nucleic acid supplied to an organism or receptacle to be inserted or recombined wholly or partially into the target sequence either by DNA repair mechanisms, homologous recombination (HR), or by non-homologous end-joining (NHEJ).

### Duration

“Duration” as used herein means the period of time during which an activity or event continues. In certain embodiments, the duration of treatment is the period of time during which doses of a pharmaceutical agent or pharmaceutical composition are administered.

### Expression Vector

“Expression vector” as used herein means any nucleic acid designed to artificially encode an exogenous protein or proteins in a host cell. Examples for expression vectors comprise plasmid DNA, T-DNA, Virus—RNA, ssDNA or dsDNA, Replicons, autonomously replicating vectors, linear ssDNA, linear dsDNA, phi polymerase products, RNA transcript, circular RNA, and in some applications of this invention, genomic and organellar DNA transferred into the host cell.

### Fragment

“Fragment” is used herein to indicate a non-full length part of a nucleic acid or polypeptide. Thus, a fragment is itself also a nucleic acid or polypeptide, respectively.

### Gene

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

### Gene Targeting

“Gene targeting” is used herein as any genetic technique that induces a permanent change to a target nucleic acid sequence including deletion, insertion, mutation and replacement of nucleotides in a target sequence.

### Genomic Modification

“Genomic modification” is used herein as any modification generated in a genome or a chromosome or extra-chromosomal DNA or organellar DNA of an organism as the result of gene targeting or gene-functional modification.

### Host Cell

“Host cell” used herein may be a naturally occurring cell or a transformed cell that may contain a vector. Host cells may be cultured cells, explants, cells in vivo, and the like. Host cells may be prokaryotic cells such as E. coli, or eukaryotic cells such as plant, yeast, insect, amphibian, or mammalian cells, such as CHO and HeLa.

According to some embodiments, said host cell is a whole or partial, differentiated or undifferentiated, cell in organism, organ, tissue or callus.

### Identity

“Identical” or “identity” as used herein in the context of two or more nucleic acids or polypeptide sequences mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

### Inhibit

“Inhibit” as used herein may mean prevent, suppress, repress, reduce or eliminate.

### In-Vitro

“In-vitro” is defined herein as an artificial environment outside the membranes of a whole or partial, differentiated or undifferentiated, living organism, organ, tissue, callus or cell. In some embodiments, the term in-vitro is not inside a viable cell.

### In-Vivo

“In-vivo” is defined herein as inside a whole or partial, differentiated or undifferentiated, organism, organ, tissue, callus or cell.

### Kits

A kit as used herein may comprise the compositions described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

### Label

“Label” as used herein means a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

### Mismatch

“Mismatch” means a nucleobase of a first nucleic acid that is not capable of pairing with a nucleobase at a corresponding position of a second nucleic acid.

### Modified Oligonucleotide

“Modified oligonucleotide” as used herein means an oligonucleotide having one or more modifications relative to a naturally occurring terminus, sugar, nucleobase, and/or internucleoside linkage.

### Modulation

“Modulation” as used herein means a perturbation of function and/or activity and/or structure. In certain embodiments, modulation means an increase in gene expression. In certain embodiments, modulation means a decrease in gene expression.

### Mutant

“Mutant” as used herein refers to a sequence in which at least a portion of the functionality of the sequence has been lost, for example, changes to the sequence in a promoter or enhancer region will affect at least partially the expression of a coding sequence in an organism. As used herein, the term “mutation,” refers to any change in a sequence in a nucleic acid sequence that may arise such as from a deletion, addition, substitution, or rearrangement. The mutation may also affect one or more steps that the sequence is involved in. For example, a change in a DNA sequence may lead to the synthesis of an altered mRNA and/or a protein that is active, partially active or inactive.

### Nucleic Acid

“Nucleic acid sequence” or “oligonucleotide” or “polynucleotide” as used herein mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH2, NHR, NR2 or CN, wherein R is C1-C6 alkyl, alkenyl or alkynyl and halo is F, Cl, Br or I. Modified nucleotides also include nucleotides conjugated with cholesterol through, e.g., a hydroxyprolinol linkage. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver. Mixtures of naturally occurring nucleic acids and analogs may be made; alternatively, mixtures of different nucleic acid analogs, and mixtures of naturally occurring nucleic acids and analogs may be made.

### Operably Linked

“Operably linked” used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

### Promoter

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, CaMV 35S promoter, NOS promoter, heat-shock promoters, Steroid-regulated promoters, Metal-regulated promoters, Seed promoters and plant ubiquitin promoters.

### Recombinant Host Cells

“Recombinant host cells” refers to cells which have been transformed with vectors constructed using recombinant DNA techniques.

### Selectable Marker

“Selectable marker” used herein may mean any gene which confers a phenotype on a host cell, tissue, organ, callus or organism in which it is expressed to facilitate their identification and/or selection of those which are transfected or transformed with a genetic construct. Representative examples of selectable markers include the ampicillin-resistance gene (AmpR), tetracycline-resistance gene (TcR), bacterial kanamycin-resistance gene (KanR), zeocin resistance gene, the AURI-C gene which confers resistance to the antibiotic aureobasidin A, phosphinothricin-resistance gene (Bar), neomycin phosphotransferase gene (nptII), hygromycin-resistance gene, beta-glucuronidase (GUS) gene, chloramphenicol acetyltransferase (CAT) gene, green fluorescent protein (GFP)-encoding gene and luciferase gene. In some embodiments of this invention a selectable marker can be produced from a modification of an endogenous gene, for example abolishment of a chemokine receptor expressed and displayed on the surface of a cell when a mutation of this gene results in a frame-shift mutation and can then be negatively selected with an antibody, or for example a W568L mutation in the Tobacco Acetolactate synthase gene which results in resistance the herbicides chlorsulfuron and imazaquin.

### Stringent Hybridization Conditions

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength and pH. The Tₘ may be the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium).

Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

### Complementary

“complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

### Substantially Identical

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

### Target Nucleic Acid

“Target nucleic acid” or “target sequence” as used herein is any desired predetermined nucleic acid sequence to be acted upon, including but not limited to coding or non-coding sequences, genes, exons or introns, regulatory sequences, intergenic sequences, synthetic sequences and intracellular parasite sequences. In some embodiments, the target nucleic acid resides within a target cell, tissue, organ or organism. The target nucleic acid comprises a target site, which includes one or more nucleotides within the target sequence, which are modified to any extent by the methods and compositions disclosed herein. For example, the target site may comprise one nucleotide. For example, the target site may comprise 1-300 nucleotides. For example, the target site may comprise about 1-100 nucleotides. For example, the target site may comprise about 1-50 nucleotides. For example, the target site may comprise about 1-35 nucleotides. In some embodiments, a target nucleic acid may include more than one target site, that may be identical or different.

### Targeted Gene-Functional Modification

“Targeted gene-functional modification” and “target gene modification” are directed to any genetic technique that results in a permanent or temporary alteration in a target nucleic acid, including but not limited to deletion, insertion, mutation, replacement, nicking, methylation, acetylation, ligation, recombination, helix unwinding, chemical modification, labelling, activation, inactivation and repression of one or more nucleotides in a target sequence.

### Therapy

“Therapy” as used herein means a disease treatment method. In certain embodiments, therapy includes, but is not limited to, chemotherapy, surgical resection, transplant, and/or chemoembolization.

### Transgenic Organism

The term is directed to an organism having one or more target gene modification(s) in it's genome, introduced by the compositions and methods disclosed herein. For example, modification is selected from: insertion, mutation, replacement of one or more nucleotides, nicking, methylation, acetylation, ligation, recombination, helix unwinding, chemical modification, labelling, activation, inactivation and/or repression. The organism may be any type of organism, such as, human, animal, plant, and the like.

### Transient Expression

“Transient expression” or “transiently expressing” used herein may refer to the transcription, or translation from a provided nucleic acid in a whole or partial, differentiated or undifferentiated, organism, organ, tissue, callus or cell, said expression being limited due to non-integration of the provided nucleic acid into the stable nucleic acids of the organism, organ, tissue, callus or cell comprising the genome or organellar nucleic acids. Vectors for transient expression comprise provided linear or circular ssDNA, dsDNA or RNA, plasmids, autonomously replicating vectors, viruses, in-vitro transcripts, T-DNA, synthetic nucleic acids and modified derivatives thereof. Thus, while transient expression is non-hereditable by definition, it may be expressed continuously in cell lineages and autonomously transferred from cell to cell due to nucleic acid replication outside of a chromosome or an organellar-genome.

### Treat

“Treat” or “treating” used herein when referring to protection of a subject from a condition may mean preventing, suppressing, repressing, or eliminating the condition. Preventing the condition involves administering a composition described herein to a subject prior to onset of the condition. Suppressing the condition involves administering the composition to a subject after induction of the condition but before its clinical appearance. Repressing the condition involves administering the composition to a subject after clinical appearance of the condition such that the condition is reduced or prevented from worsening. Elimination of the condition involves administering the composition to a subject after clinical appearance of the condition such that the subject no longer suffers from the condition.

### Variant

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

### Vector

“Vector” as used herein means a nucleic acid sequence used for the purpose of nucleic acid delivery. A vector may be used in this invention to bring about genetic transformation, the expression of a protein, the transcription of an RNA, or to be used directly as a Donor for homologous recombination or non-homologous end-joining. A vector may be a plasmid DNA, T-DNA, Virus—RNA, ssDNA or dsDNA, Replicons, autonomously replicating vectors, linear or circular ssDNA, linear or circular dsDNA, branched phi polymerase products, nucleic acid dendrimers, RNA transcript, circular RNA, bacteriophage, bacterial artificial chromosome or yeast artificial chromosome and in some applications of this invention, genomic and organellar DNA transferred into the host cell. A vector may be either non-replicating, a self-replicating extrachromosomal vector or a vector which integrates into a host genome.

### Wild Type

As used herein, the term “wild type” sequence refers to a coding, a non-coding or an interface sequence which is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes.

According to some embodiments, the composition comprising the programmable molecular complex which comprises a protein (polypeptidic) moiety, and a nucleic acid moiety, self-assemble in-vivo in a living cell, organism, tissue, callus, organ or part thereof, in the presence of a target nucleic acid sequence(s) to form an active, programmed functional molecular complexes.

According to some embodiments, the various programmed molecular complexes can be constructed to permanently or transiently modify an existing or imminent eukaryotic, prokaryotic, synthetic, intracellular parasite or viral target sequence such as that found in a genome, a nucleus, a chromosome, a cytoplasm, an organelle, or an extra-chromosomal nucleic acid. The target modification performed by the action of the molecular complex comprises heritable and non-heritable, permanent and transient genetic changes/modifications. In some embodiments, the target is comprised of a nucleic acid involved in a genetic trait of interest which would be advantageous to alter. Alterations in the targeted sequence include, for example, but not limited to: permanent deletion, mutation, insertion of nucleic acids, and replacement of a targeted sequence with another nucleic acid sequence, knocking-out, frame-shifting, or any change in any fashion of the transcription or translation of a gene, its regulatory sequences, the genes regulating the gene of interest or their regulatory sequences in a regulatory chain of events. Permanent changes to the target nucleic acid include, for example, genetic material editing or sequence alterations such as nucleic-acid mutation, deletion, insertion, replacement and recombination. Transient changes to the target sequence include, for example, binding, digestion, nicking, helix unwinding, activation, inactivation, chemical modification, methylation, acetylation and labelling of the target nucleic acid. Target modification include, for example, target functional modification which can lead in the cell to changes in transcriptional activation, transcriptional inactivation, RNA silencing, alternative RNA splicing, chromatin rearrangement, intracellular parasite inactivation, and changes in cellular localization or compartmentalization of the target nucleic acid.

According to some embodiments, and without wishing to be bound to any theory or mechanism, the design of the programmable molecular complex, is based on its ability to self-assemble, its ability to target a predefined intended sequence on a target nucleic acid, and its ability to act upon the target sequence in a predetermined fashion. The components of the complex are modular and adjustable to be suitable for 1) particular types of molecular action required, 2) the target, and 3) the desired nucleic acid delivery method used for its expression in-vivo. The methods and compositions of the present disclosure have several advantages over other systems known in the art. For example, the protein moieties of the complex are in-active as monomers, and only correct spacing, within a limited range, of the two SCNA oligonucleotides that bind the target nucleic acid at a predetermined sequence, will result in placement of the effector domains of the protein moieties such that they dimerize and are able to specifically act upon the desired, predetermined target site. Such a setting, whereby only dimers of programmed molecular complexes (i.e. complex which comprises a protein moiety linked to the SCNA, which is bound to the target nucleic acid), reduces or completely eliminates potential off-site or non-specific cleavage, since the protein moiety by itself does not bind the target nucleic acid and does not act as a monomer.

According to some embodiments, the active portion (functional domain), of the molecular complex is designed to be activated only upon dimerization of the functional domain of the protein moiety. The unprogrammed protein component is designed to have low or practically no non-specific affinity to nucleic acid sequence and to the target site. Thus, while for all types of modifications a single type of monomer of the protein moiety needs to be expressed, for the minimal functions of point modification, such as, for example, a point mutation mediated by a nuclease domain, or alternatively, a point methylation mediated by a methylase domain, two SCNAs, designed to bind sequences flanking the target site, should be present to affect the correct spacing of the proteins and allow both their binding and their dimerization with each other. This advantageously enhances the sequence-specificity of the complex. In some embodiments, for the editing functions of deletion and replacement, two different sites flanking the region of interest may need to be cleaved concomitantly. In such embodiment, even in this case, only one exogenous protein component needs to be expressed along with four SCNAs. When the oligonucleotides are depleted, either by dilution or by degradation, the unprogrammed expressed protein has no affinity to the target nucleic acid and will cease to act upon it (i.e, in this case, cease cleaving the target nucleic acid).

According to some embodiments the protein (polypeptide) moiety may be expressed as separate polypeptides or as one contiguous protein (polypeptide). In some embodiments, the protein moiety (component) may have one or more identifiable domain(s), identifiable according to structure and/or function (utility). In some embodiments, one structural domain may have more than one utility domain, that is, a separable structural domain may have several functions. According to some embodiments, the protein moiety may comprise one or more of the following structural and/or utility domains: a) an “effector domain” (functional domain), that can interact with and consequently affect the target nucleic acid; and/or b) a “linking domain”, that can directly or indirectly specifically bind the SCNA; and/or c) a “cellular localization domain”; and/or d) interdomain connectors or spacers; and any combination thereof.

According to some embodiments, the “Effector Domain” (also termed herein as “Functional Domain”), interacts with the target nucleic acid after assembly of the molecular complex and exert the intended effect on the target sequence. In some exemplary embodiments, this domain has an enzymatic or catalytic function, comprising a nucleic acid modifying activity. In some embodiments, this domain may be derived from active domains derived from whole, or portions of, or modified portions of proteins of known function such as, a DNA binding protein, a nuclease, a methylase, a methylated DNA binding factor, a transcription factor, a chromatin remodelling factor, a polymerase, a demethylase, an acetylase, a deacetylase, a kinase, a phosphatase, an integrase, a recombinase, a ligase, a topoisomerase, a girase and a helicase. In some embodiments, the functional domain may be constructed by fusing amino-acid sequence(s) of active domains derived from whole, or portions of, or modified portions of proteins of known function comprising a DNA binding protein, a nuclease, a methylase, a methylated DNA binding factor, a transcription factor, a chromatin remodelling factor, a polymerase, a demethylase, an acetylase, a deacetylase, a kinase, a phosphatase, an integrase, a recombinase, a ligase, a topoisomerase, a girase and a helicase. In some embodiments, for an effector domain which is or is derived from a nuclease, the DNA-binding recognition domain of the nuclease may be removed. For example, when the effector domain is derived from a FokI nuclease, the FokI site recognition and binding domains are absent in the Effector domain of the protein moiety. In some embodiments, the effector domain is devoid of a specific target-nucleic acid binding site, i.e., it cannot specifically bind a specific target sequence.

According to some embodiments, the “Linking Domain” is designed to directly or indirectly specifically bind/attach the SCNA (and in particular, to the SCNA recognition region). The binding/attachment between the linking domain and the SCNA can be direct, or indirect through, for example, a modification on the SCNA. The attachments/binding/linking between the linking domain and the SCNA enables in vivo the assemblage of the SCNA with the protein moiety. In some embodiments, the linking domain is constructed by fusing the amino-acid sequence of the protein moiety to amino-acids incorporating a domain which specifically binds a nucleotide sequence or a chemical or a biological element on the specificity-conferring nucleic acid. The physical interaction between the Linking Domain and the Specificity Conferring Nucleic Acid can be due to, but is not limited to, an affinity due to one or more of the following types of interactions; ligand-receptor, ligand-substrate, Hydrogen bonds, van der Waals bonds, Covalent bonds formed in-vivo, Ionic bonds and hydrophobic interactions. Non-covalent binding examples comprise, one or more, or of fragments or portions or modified forms of the following: binding-pair examples: Biotin-Avidin; Biotin-Streptavidin; Biotin-modified forms of Avidin; Protein-protein; nucleic acid-protein; ligand-receptor; substrate-ligand; antigen-antibody; antigen-single chain antibody; hapten—antibody or -single chain antibody; hormone-hormone binding protein; agonist-receptor; receptor antagonist-receptor; protein A—IgG; enzyme cofactor—enzyme; enzyme inhibitor-enzyme; single-strand DNA-VirE2; dsDNA-StickyC; RNA—Argonaute family protein; dsRNA-RnaseIII family protein; nucleic acid-viral coat protein and Agrobacterium VirD2 or parts thereof—VirD2 binding protein, whereby each of the Specificity-Conferring Nucleic Acid and Linking Domain comprise one of the pair members. In an exemplary embodiment, the Linking Domain contains a single chain antibody ScFV capable of binding the dye Fluorescein which in turn is chemically linked via a linker to a 5′-terminus or a 3′-terminus of the Specificity-Conferring Nucleic Acid, thus enabling the association of the protein moiety and the nucleic acid moiety of the programmable complex. In some embodiments, the Linking Domain is derived from the C. elegans PUF5 binding element eight triple-helical repeat, and the Specificity-conferring nucleic acid (SCNA) contains the RNA sequence as set forth in SEQ ID NO:1 (CUCUGUAUCUUGU) at or sufficiently near one of its termini. In this embodiment the protein and SCNA are directly brought together without the need for a chemical modification on the SCNA, permitting its biosynthesis in-vivo as a transcript and thus enabling the in-vivo association of the protein moiety and the nucleic acid moiety of the programmable complex. In some exemplary embodiments, an RNA sequence/molecule capable of forming secondary or tertiary structures (such as hairpin loop), located within the SCNA, interacts with the linking domain of the protein moiety, which is an RNA-motif-binding Linking Domain, derived from the viral TAT proteins (such as, HIV, BIV, and the like). In some exemplary embodiments, a 20-mer boxB RNA hairpin binding sequence from bacteriophage Phi21 is located on the SCNA and is capable of binding/attaching it's counterpart linking Domain on the protein moiety, which is derived from the RNA-binding protein (RBP) bacteriophage Phi21 NProtein. In another exemplary embodiment, which allows the production of the SCNA in-vivo, the Linking Domain is derived from a protein which binds Agrobacterium VirD2 protein, comprising VirD2-binding proteins found in bacteria comprising VBP1, VBP2 and VBP3 and artificial single chain antibodies designed to bind VirD2. In this embodiment, the SCNA is produced as a ssDNA from a T-DNA in an Agrobacterium, where it is covalently bound at its 5′-terminus to tyrosine 29 of VirD2 which is required for the covalent association, whereby the covalent binding occurs in-vivo. The catalysis occurs in the bacterium and the complex is subsequently exported from the bacterium through a bacterial secretion system into a eukaryotic cell comprising whole or partial plant-, animal- and human—cells, tissues, calli and organs. In this embodiment, the VirD2-binding domain in the Linking Domain binds the VirD2 protein attached to the SCNA thus enabling the association of the protein moiety and the nucleic acid moiety of the programmable complex. In this embodiment, modifications to VirD2 expressed in the bacterium could be designed that would decrease DNA integration and could be of benefit to avoid non-specific DNA integration. Examples of Covalent binding formed in vivo in the target organism, comprise, respectively, on the recognition region of the SCNA, and in the Linking domain, one or more, of fragments or portions or modified forms of, but not limited to, the following binding-pair example paired by a dash symbol; the RB sequence of T-DNA GTTTACCCGCCAATATATCCTGTCA (SEQ ID NO: 2)—Agrobacterium VirD2; Picornavirus RNA—VPg; DNA—Topoisomerase; PhiX174 phage origin sequence on ssDNA—PhiX174 phage A protein or PhiX A* protein, and the like. In one exemplary embodiment of such an in-vivo SCNA-Linking Domain attachment, a synthetic ssDNA oligonucleotide containing an RB sequence at or near its 5′-terminus is delivered to a cell where it encounters the protein moiety. The protein harbours a portion of VirD2 capable of cleaving the RB sequence and subsequently binds the rest of the oligonucleotide containing the sequence TCA at its 5′ end, an appropriate spacer, and a target-base-pairing sequence, thus effectively “programming” the molecular complex in-vivo. In some embodiments, the linking domain is devoid of a specific target-nucleic acid binding site, i.e., it cannot specifically bind a specific target sequence.

According to some embodiments, a “cellular localization domain” which can localize the protein moiety or the programmed protein moiety or the assembled complex to a specific cellular or sub cellular localization in a living cell, may optionally be part of the protein moiety. The cellular localization domain may be constructed by fusing the amino-acid sequence of the protein moiety to amino-acids incorporating a domain comprising a Nuclear localization signal (NLS); a Mitochondrial leader sequence (MLS); a Chloroplast leader sequence; and/or any sequences designed to transport or lead or localize a protein to a nucleic acid containing organelle, a cellular compartment or any subdivision of a cell. In some exemplary embodiments, the organism is eukaryotic and the cellular localization domain comprises a nuclear localization domain (NLS) which allows the protein access to the nucleus and the genomic DNA within. The sequence of said NLS may comprise any functional NLS positively charged sequence comprising, for example, the SV40NLS sequence PKKKRKV (SEQ ID NO: 3). In another exemplary embodiment, this domain is comprised of a leader sequence enabling the entry of the protein moiety or of the programmed nucleo-protein into an organelle, enabling the desired modification of the organelle DNA by the complex. In another exemplary embodiment, a sequence derived from the Yeast mitochondrial Cox4p (MLSLRQSIRFFKPATRTLCSSRYLL (SEQ ID NO: 4)) or a sequence derived from the human malate dehydrogenase mitochondrial leader sequence (MLS) (MLSALARPASAALRRSFSTSAQNNAKVAVLGAS (SEQ ID NO: 5)) or derived from the Arabidopsis Lipoic acid synthase (NCBI Ref. Seq. ID: NP_179682.1 designated herein as SEQ ID NO: 6: MHSRSALLYRFLRPASRCFSSSS) may be used to localize the complex into a mitochondrial matrix to modify mitochondrial DNA. One use of this application may include the curing of maternally inherited mitochondrial DNA defects in various Eukaryotes, such as Chronic Progressive External Ophthalmoplegia Syndrome in Humans. Another example is inducing defects to bring about male sterility in plants used for hybrid plant production. In one embodiment the mitochondrial target is an ATPase and reconstitutes the function of the pcf locus in Petunia.

According to further embodiments, optional various interdomain connectors or spacers designed to allow the desired function of the complex by serving as molecular adapters or hinges. Many such connectors may be foreseen by those skilled in the art. Choice of connector may affect the specificity of the programmed molecular complex by affecting the range of target nucleic acid in reach of the functional domain active site. In one exemplary embodiment, the C′ of the Linking Domain and the N′ of the Functional Domain are flexibly connected with the amino acids GGSGG (SEQ ID NO: 7), spanning about 15 Angstrom. In another embodiment, a rigid Alpha-helix linker with the amino acids NIHHVTWHMDFP (SEQ ID NO: 8) spanning about 16 Angstrom is used. In another embodiment, a rigid helical linker with the amino acids PNSLIVP (SEQ ID NO: 9) spanning about 16.88 Angstrom is used. In another embodiment, a disordered coiled linker with the amino acids TGLDSP (SEQ ID NO: 10) spanning about 15.55 Angstrom is used. Extra amino acids encoded by restriction enzyme sites may be added in the interdomain connector to facilitate exchanging protein modules (e.g. GSLE (SEQ ID NO: 11) encoding BamHI/XhoI).

According to some embodiments, the nucleic-acid moiety of the molecular complex, termed herein “Specificity-conferring nucleic acid” (SCNA) or “programming nucleic acid” comprises one or more portions (regions) and functions. One portion (region) defines the target region to be acted upon, and contains the specificity-defining sequence. The specificity-defining sequence in the SCNA defines its specificity to the target nucleic acid by base pairing. This pairing may form, for example, but not limited to: a full or partial double helix, a full or partial triple helix, D-loops and branched forms, and may be the result of hydrogen bonds or Hoogsteen hydrogen bonds or combinations thereof. In some embodiments, the specificity-defining sequence is capable of interacting with the target nucleic acids, at regions which are proximate to, or flanking the target site. In some embodiments, the specificity-defining sequence of the SCNA does not bind/interact with the target site. In some embodiments, the specificity-defining sequence may include any number of nucleotides. For example, the specificity-defining sequence may be at a length of about 3-200 nucleotides. For example, the specificity-defining sequence may be at a length of about 10-100 nucleotides. For example, the specificity-defining sequence may be at a length of about 15-50 nucleotides. For example, the specificity-defining sequence may be at a length of over about 18 nucleotides.

According to some embodiments, a second portion of the SCNA, is the recognition region (portion), which is a region that can specifically bind/attach/recognize the linking domain of the protein moiety. In some embodiments, this recognition region may be and/or include a modification or a Linking-Domain-recognition sequence (also termed herein as SCNA nucleotide motif or SCNA linking domain-binding nucleotide sequence). The recognition region may be an integral part or may be linked (for example, covalently) to the specificity-defining sequence, and may be composed of a sequence or a modification which enables the binding of the SCNA to the Linking Domain of the protein moiety, as detailed above.

In some embodiments, the SCNA is comprised of but not limited to, a molecule of the following types: single-strand DNA, single strand RNA, double strand RNA, modified DNA, modified RNA, locked-nucleic acid (LNA), peptide-nucleic acid (PNA) and any combinations of the above. In some embodiments, the SCNA may additionally include one or a multiplicity of modifications which may enhance its stability, enhance its specificity to the target, modify its affinity to nucleic acids and/or enable its binding to the Linking Domain of the complex. The modifications may be positioned at its 5′ end, at its 3′ end, as spacers and/or internally on the SCNA. Exemplary modifications include, but are not limited to, Nucleotides, Biotin, Fluorescein, Amine-linkers, oligo-peptides, Aminoallyl, a dye molecule, fluorophores, Digoxygenin, Acrydite, Adenylation, Azide, NHS-Ester, Cholesteryl-TEG, Alkynes, Photocleavable Biotin, Thiol, Dithiol, Modified bases, phosphate, 2-Aminopurine, Trimer-20, 2,6-Diaminopurine, 5-Bromo-deoxiUridine, DeoxiUridine, Inverted dT, dideoxi-nucleotides, 5-methyl deoxyCytidine, deoxyInosine, 5-nitroindole, 2-O-methyl RNA bases, Iso-dC, Iso-dG, Flourine modified bases, Phosphorothioate bonds and the Agrobacterium VirD2 protein and parts of said VirD2 and modifications of VirD2.

According to some embodiments, the SCNA may further include optional spacer sequences that may be used for optimizing the molecular distances and degrees of freedom necessary to bring together the linking domain and a target nucleic acid. In some embodiments, the spacer sequences may be at a length of about 0-100 nucleotides. For example, the spacer may be at a length of about 0-6 nucleotides.

According to some embodiments, the SCNA may be produced chemically and/or biologically, in-vitro and in-vivo, and the modification may be pre-synthesized or added post-synthesis. In some exemplary embodiments, the SCNA is produced chemically and is composed of phosphorothioate-modified ssDNA which is modified at one of its termini by the linking of a C6-Fluorescein dye molecule. This SCNA is consequently delivered to a cell, (for example, by particle bombardment, Polyethylene glycol transfection, liposomes, viral particles, silicon-carbide whiskers and/or electroporation) where it encounters both the protein component of the molecular complex, which comprises a Linking Domain comprising a single chain antibody ScFV capable of binding the dye Fluorescein, thus programming the molecular complex, and delivering/targeting the complex to its intended target nucleotide sequence. According to some embodiments, the SCNA does not bind/interact with the target site.

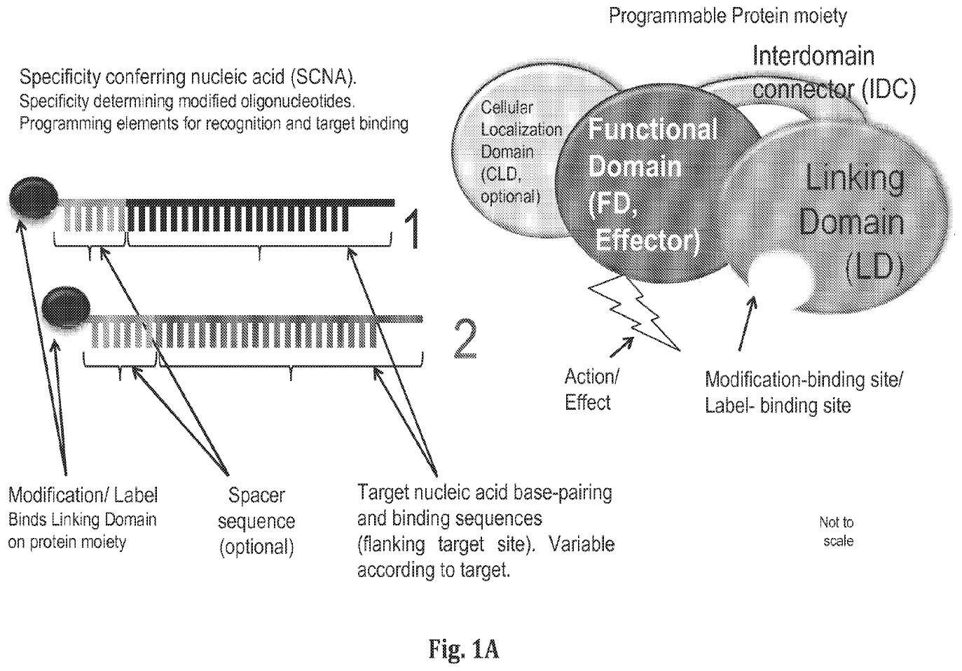

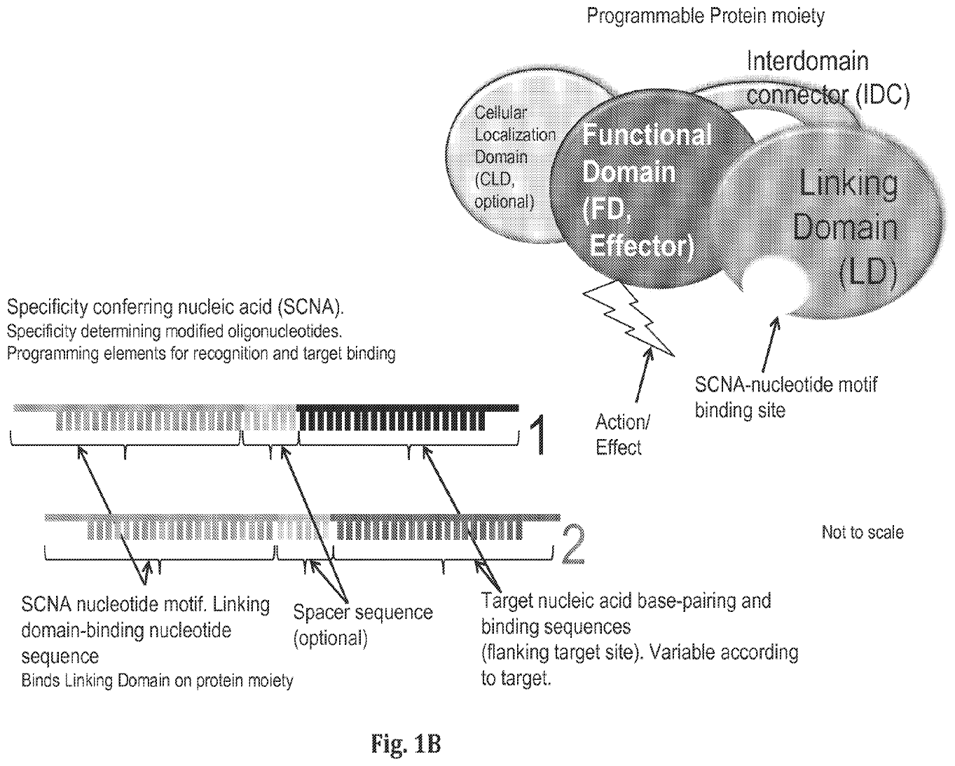

Reference is now made to FIGS. 1A-B which are schematic cartoons (not to scale) showing elements/components of a programmable molecular complex, according to some embodiments. The schematic cartoons (not to scale) of FIGS. 1A-B, show a molecule of a programmable protein moiety as a monomer, and two molecules of specificity conferring nucleic acids (SCNA). As shown in FIGS. 1A-B, the protein moiety is a polypeptide (a chain of amino-acids) arranged into several structural/functional domains: a linking domain (LD), a functional or Effector Domain (FD); an optional Cellular Localization Domain (CLD) and an optional interdomain connector(s) (IDC), each defined by their role in the molecular complex. The function of the linking domain is to bind the SCNA. The function of the Effector domain is to interact with the target nucleic acid and structurally modify the target site and/or modify it's function and/or the function of the entire target nucleic acid. The function of the optional cellular localization domain is to localize the protein complex to the same cellular or subcellular compartment as the target nucleic acid. The function of the optional interdomain connectors is to allow optimal molecular distances and degrees of freedom between domains for the proper function of the complex. The SCNAs are comprised of a nucleic acid chain or a modified nucleic acid chain (comb shape) and include a modification, preferably at one of its termini (shown in FIG. 1A as black oval) for binding to the protein moiety, or a sequence, (termed the SCNA-nucleotide motif, or Linking-domain-binding nucleotide sequence or Linking-Domain-recognition sequence or segment, shown in FIG. 1B, arrow marked comb), which can bind the linking domain on the protein moiety. In the non-limiting example presented in FIGS. 1A-B, the specificity determining portion of the SCNA is single stranded. In some embodiments, the SCNA may form double strand segments/regions (by self annealing, such as forming hairpin loops). The specificity of the SCNA to a predetermined target nucleic acid sequence is achieved through a stretch of base-pairing nucleic acids or modified nucleic acids (Target nucleic acid base-pairing, comb shape), also termed the variable sequence, which may include any number of nucleotides, such as, 3-200 nucleotides, and any ranges thereof. For example, the length can be 10-100 nucleotides. For example, the length can be at least 18 nucleotides. Optional spacer sequences (Spacer sequence, comb shape), may be present for optimizing the molecular distances and degrees of freedom necessary to bring together the linking domain and a target nucleic acid. In some embodiments, the spacer sequences may be at a length of about 0-100 nucleotides. For example, the spacer may be at a length of about 0-6 nucleotides. Action or effect of the functional domain of the protein moiety, which occurs upon binding to the SCNA linking domain and dimerization and its consequent co-localization to the target nucleic acid, is portrayed as a lightning symbol (“Action/Effect”).

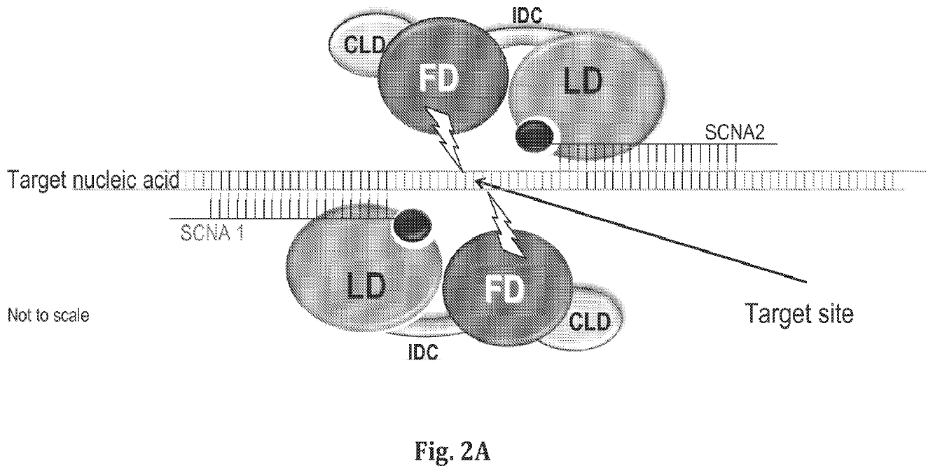

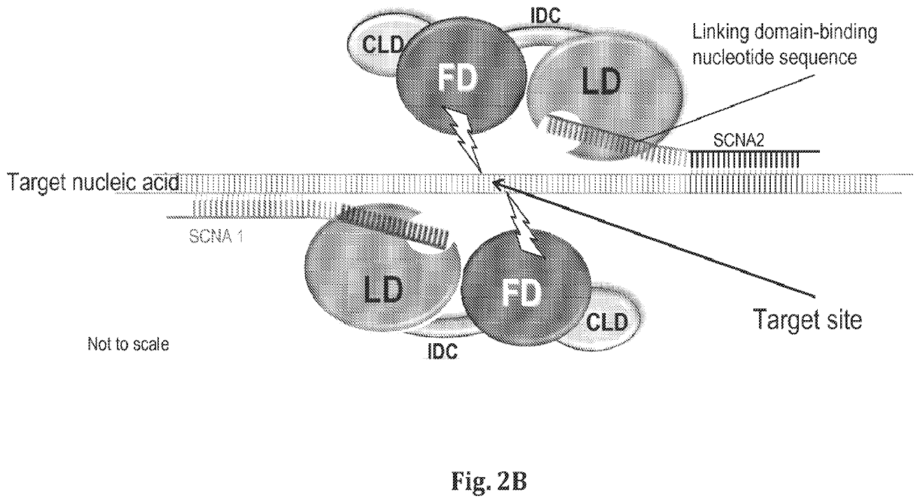

Reference is now made to FIGS. 2A-B, which are schematic cartoons showing the assembly of the programmable molecular complex, according to some embodiments. The schematic cartoons (not to scale) of FIGS. 2A-B demonstrate mode of assembly of the components of the programmable molecular complex on a target nucleic acid. In the example shown in FIGS. 2A-B, two protein monomers bind two different SCNAs, each having a different specificity determinant in its variable sequence region. These SCNAs base pair and bind with predefined homologous sequences on a target nucleic acid (marked in the Figs. as “Target nucleic acid”). This base pairing can form a double- or a triple-helix with the Target nucleic acid, depending whether the target is double- or single-stranded (illustrated in these figures, as dsDNA). Both SCNAs can bind either the same strand or opposite strands as required, in an optimized distance. The SCNAs can bind the protein Linking Domain through a modification on their terminus (FIG. 2A) or through an SCNA-nucleotide motif (FIG. 2B). Upon assembly the Functional Domain prompts its effect on the predetermined target site (marked as “Target site”) on the target nucleic acid.

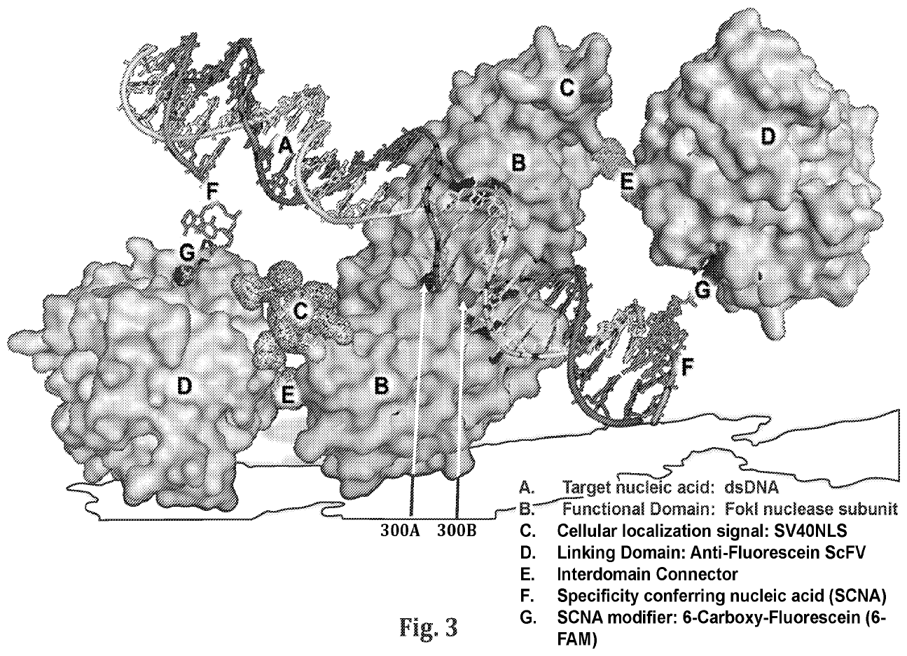

Reference is now made to FIG. 3, which demonstrates a 3D Modeled example of a molecular complex designed for cleavage of a predefined nuclear dsDNA target sequence, according to some embodiments. A programmed dimerized protein moiety is shown in association with its Target dsDNA (A, shown in part). Each monomer of the protein moiety is comprised of a Functional Domain derived from a FokI nuclease subunit (B); a cellular localization domain derived from the SV40NLS (C); a Linking Domain derived from an anti-Fluorescein single-chain variable fragment antibody (anti-FAM ScFV, D) and an interdomain connector (E). Each Linking Domain (D) is shown bound to a Specificity Conferring Nucleic Acid, SCNA ssDNA (F, shown in part) through its modifier 6-carboxy Fluorescein molecule (G), which is covalently bound to the terminus of each SCNA. Expected cleavage sites (target site) of the target dsDNA (shown as balls on the helix backbone) are marked with arrows 300A-B. Each SCNA is depicted here as forming a partial triple-helix occupying the major groove of the dsDNA target-flanking sequence.

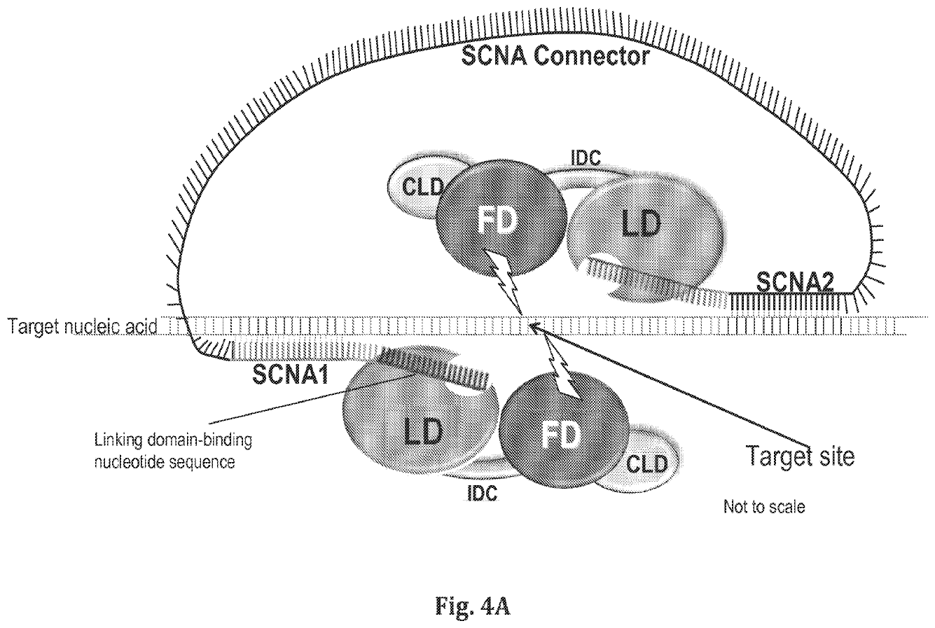

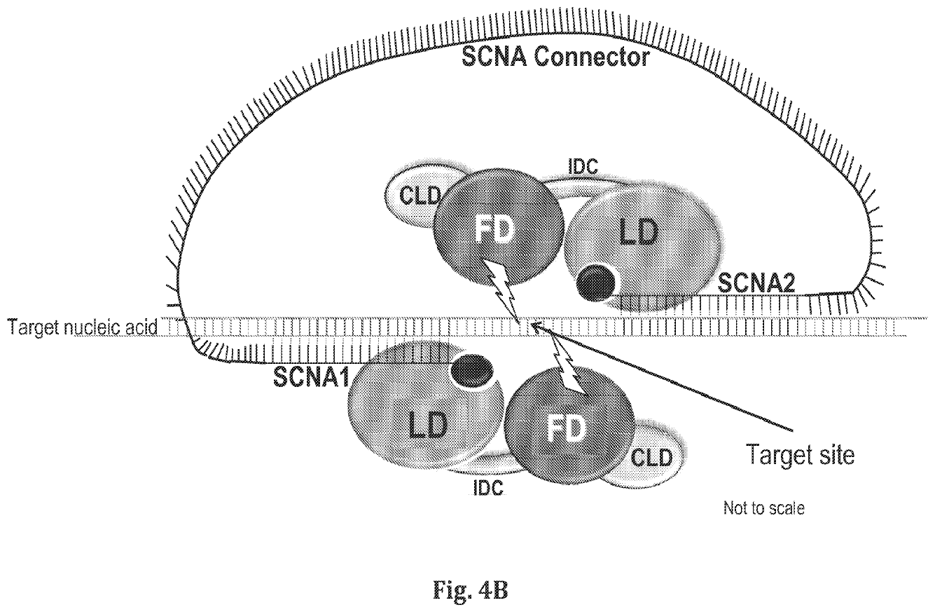

Reference is now made to FIGS. 4A-B, which are schematic drawings (not to scale) of exemplary mode of assembly of the components of the programmable molecular complex on a target nucleic acid, according to some embodiments. As shown in the non limiting examples presented in FIGS. 4A-B, two monomers of the protein moiety bind two different SCNAs (SCNA1, SCNA2), each having a different specificity determinant in the variable sequence region. As shown in the figures, both SCNAs reside on a single nucleic acid and are connected with a sequence of undetermined sequence or length which does not base-pair with the Target, referred to herein as the “SCNA connector”. The SCNA connector may include any sequence of nucleotides, at any length (X(n)). In some embodiments, X(n) signifies an undetermined length of RNA nucleotides connecting the two specificity conferring regions to each other. In some embodiments, for linear DNA, the expected optimal length (n) is about, for example, between 10-100 nucleotides. For example, the length is about 35-73 nucleotides (nts). For example, the length is over about 70 nucleotides. For example, the length is shorter than about 35 nucleotides. These SCNAs base pair and bind with predefined homologous (corresponding) sequences on the target nucleic acid. This base pairing can form a double- or a triple-helix with the target nucleic acid, depending whether the target is double- or single-stranded (in the example illustrated in FIGS. 4A-B, dsDNA). In some embodiments, both SCNAs can bind either the same strand or opposite strands of the target nucleic acid as required, in a distance optimized to achieve a desired result. In some embodiments, only one dual connected SCNA containing nucleic acid is needed to target a target site, by flanking both ends of the target site. In some embodiments, the SCNAs can bind the binding site of the Linking Domain (indentation in Linking domain) of the protein moiety, via SCNA-nucleotide motifs on both SCNAs (marked combs in Linking domain binding site, FIG. 4A, or through a modification on both termini (black ovals in Linking domain binding site, FIG. 4B). Upon assembly, the Functional Domain may prompt its effect on the target site in the target nucleic acid.

According to some embodiments, methods for delivery of the SCNA into the organism or cell comprise the multitude of methods known to those skilled in the art and are generally those optimal for the organism or cell type used in the relevant circumstance. These can include delivery of nucleic acid by the biological methods of: infection using autonomously replicating vectors, transgenic virus infection or transduction, including the use of deconstructed or partial viruses, inoculation, agrobacterium T-DNA delivery, breeding, crossing, grafting, organelle transfer, chromosome transfer, cell fusion; the chemical mediated uptake methods of: using transfection agents, DEAE-Dextran, Calcium phosphate, artificial lipids, dendrimers, polymers (PEG etc.), proteins/peptides, virus-like particles; the mechanical methods of: bombardment, injection/microinjection, pressure, whiskers; and the electrical method of electroporation, and any method that alters the cellular plasma membrane, allowing nucleic acids to actively or passively enter the cell.

According to some embodiments, methods for delivery of the nucleic acid encoding the protein module into the organism or cell comprise the multitude of methods known to those skilled in the art and are generally those optimal for the organism or cell type used in the relevant circumstance. These can include delivery of nucleic acid by crossing or breeding an organism with a transgenic organism carrying the gene or by the biological methods of: infection using autonomously replicating vectors, transgenic virus infection or transduction, including the use of deconstructed or partial viruses, inoculation, agrobacterium T-DNA delivery, grafting, organelle transfer, chromosome transfer, cell fusion; the chemical mediated uptake methods of: using transfection agents, DEAE-Dextran, Calcium phosphate, artificial lipids, dendrimers, polymers (PEG etc.), proteins/peptides, virus-like particles; the mechanical methods of: bombardment, injection/microinjection, pressure, whiskers; and the electrical method of electroporation, and any method that alters the cellular plasma membrane, allowing nucleic acids to actively or passively enter the cell.

According to some embodiments, methods for delivery of “donor DNA”, in the subgroup of uses requiring such a DNA comprising gene-insertion or gene replacement, comprise similar methods to those described for delivery of the nucleic acid which encodes the protein module. This DNA can be either single stranded, double stranded or partially double stranded, linear or circular. This DNA can be supplied on a single vector or on several vectors, concurrently or separately from the nucleic acid encoding the protein component of the molecular complex and from the specificity-determining programming nucleic acid. Thus, nucleic acids can be delivered, by choosing from the appropriate aforementioned delivery methods, to a plant or a part of a plant, to a plant tissue or organ such as an embryo, pollen, ovum, anther, stigma, whole flower, cotyledon, leaf, root, stem, petiole, to isolated plant cells such as protoplasts, or to differentiated or undifferentiated cultured plant tissue, callus, or cells. In some embodiments, nucleic acids can be delivered to a fungus, including unicellular and multicellular fungi, and to a member of the animal kingdom including invertebrates (such as arthropods and nematodes), vertebrates (such as birds, fish, mammals, reptiles, and amphibians) and to parts of these organisms including organs, cultured organs, tissues, cultured tissues, isolated cells, cell cultures, cell lines and stem cells such as human embryonic stem cells or human hematopoietic stem cells.

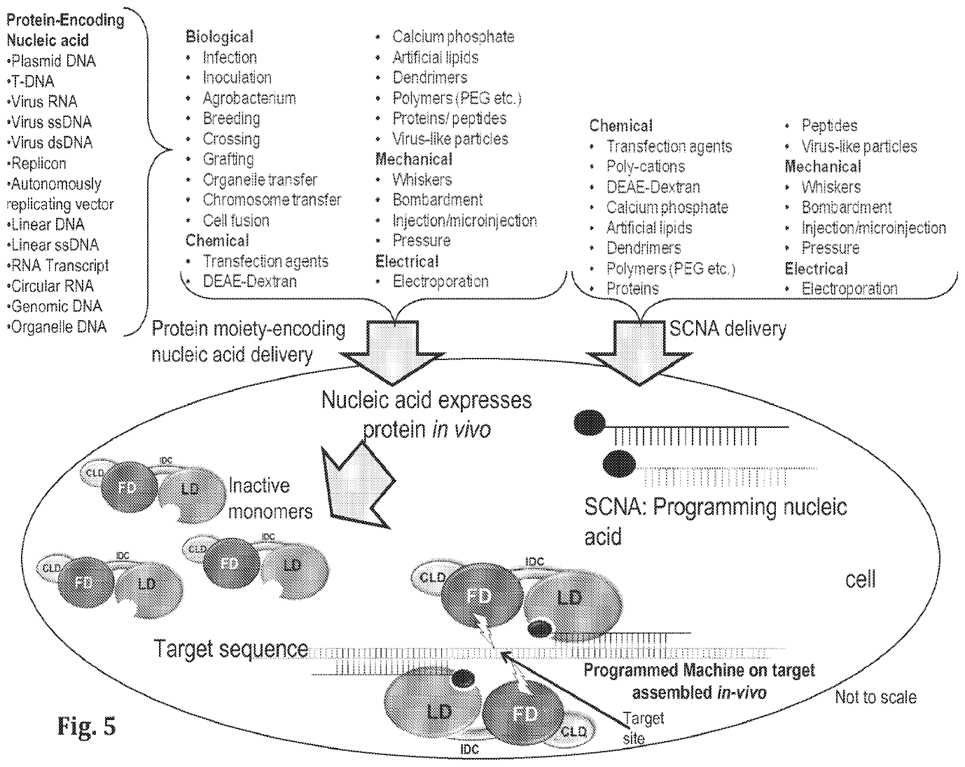

Reference is now made to FIG. 5 which shows a schematic illustration demonstrating delivery options of the programmable molecular complex to a cell using in-vitro produced SCNAs, according to some embodiments. A general scheme for selecting an appropriate delivery method is shown. A nucleic acid molecule encoding for the protein moiety is selected from the left hand column and delivered using applicable methods selected from the next two columns. A synthetic SCNA is supplied through methods selected from those shown in the two right columns. Within the target cell, a nucleic acid encoding for the protein brings about the expression of the protein by its translation in-vivo from a template RNA molecule. If the delivered nucleic acid molecule is comprised of dsDNA, it may first transcribe to RNA (via a designated promoter). If the delivered nucleic acid molecule is comprised of ssDNA it may first be complemented to dsDNA and then transcribed. If the delivered nucleic acid molecule is comprised of RNA, such as that encoding a virus or another autonomously replicating vector, it may proceed through replication via a minus strand before being translated. The translated protein can then be localized to the desired subcellular compartment, according to its localization signal (if present). The SCNAs may be delivered concomitantly or separately from the nucleic acid molecule encoding for the protein moiety by the same or different delivery method. Once the SCNA, protein moiety and Target nucleic acid are co-localized within the cell, they may assemble to form an active molecular dimeric complex. Donor DNA, if required, may also be delivered separately or simultaneously.

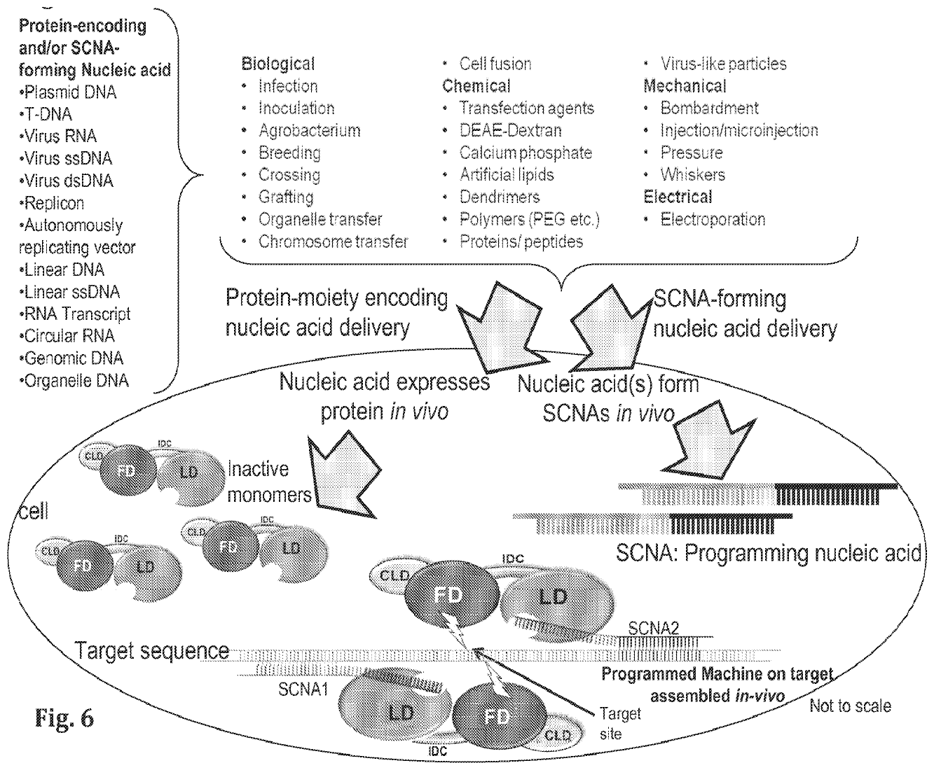

Reference is now made to FIG. 6, which is a general scheme demonstrating the delivery of the programmable molecular complex to a cell using an in-vivo produced SCNA, according to some embodiments. A nucleic acid molecule encoding the protein moiety is selected from the left hand column and delivered using applicable methods selected from the next three columns. In-vivo produced SCNAs are encoded by a nucleic acid molecule provided for that purpose and introduced into the cell using these same methods. The nucleic acid molecules encoding for the protein moiety and/or the SCNA may be delivered separately or concomitantly. In the cell, the nucleic acid encoding the SCNA expresses the SCNA by transcription or nucleic acid cleavage. If the delivered nucleic acid molecule is comprised of dsDNA, it may be first transcribed to RNA via a designated promoter. If the delivered nucleic acid molecule is comprised of ssDNA it may first be complemented to dsDNA and then transcribed. If the delivered nucleic acid molecule is comprised of RNA such as that encoding a virus or another autonomously replicating vector, it may proceed through replication via a minus strand. Within the cell, the nucleic acid encoding the protein is expressed via its translation in-vivo from an RNA molecule produced in a manner similar to that described for the SCNA. The translated protein can then be localized to the desired subcellular compartment, according to its localization signal (if present). The nucleic acid molecules encoding for the protein moiety and/or the nucleic acid molecules encoding for the SCNAs may be delivered concomitantly (at the same time) or separately, by identical or different delivery methods. Once the SCNA, protein moiety and Target nucleic acid are co-localized within the cell, they may assemble to form an active molecular dimeric complex. Donor DNA, if required, may also be delivered separately or simultaneously.

According to some embodiments, the biological synthesis in-vivo of the SCNA may be performed by several routes, such as, but not limited to: (a) the use of Agrobacterium to synthesize both nucleic acid and the Linking-Domain-binding moiety, in this example VirD2, which also catalyzes their covalent linking. Agrobacterium then facilitates the transfer to a cell of a ssDNA covalently bound to VirD2, (b) the use of Agrobacterium to transfer a T-DNA to a cell, said T-DNA comprising promoters driving the synthesis in the cell of RNA SCNAs having an RNA domain that binds the Linking Domain of the complex upon their converging. Thus, the complex, expressed in the target cell, assembles through an RNA-protein interaction, (c) the use of autonomously replicating vectors comprising viruses and viral-based expression vectors to deliver a replicon to a cell, said replicon comprising subgenomic promoters driving the synthesis of RNA SCNAs having an RNA domain that binds the Linking Domain of the complex upon their converging. Thus, the complex, expressed in the target cell, assembles through an RNA-protein interaction.

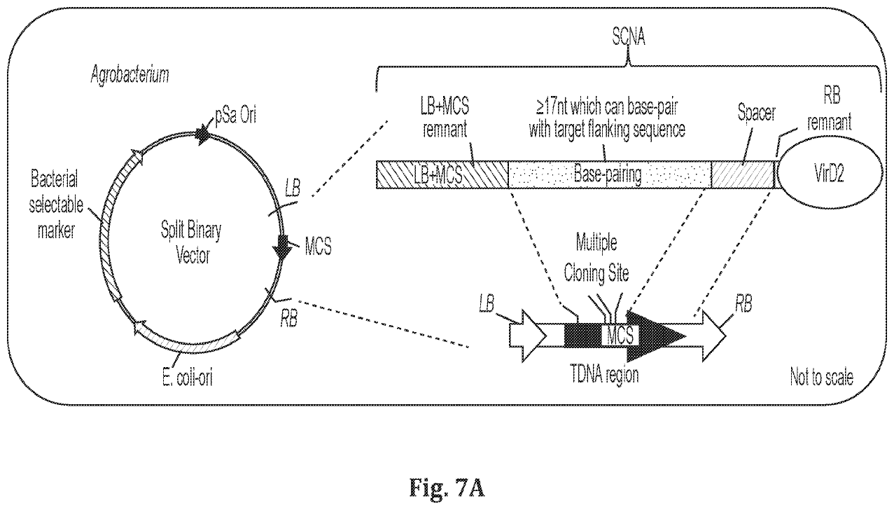

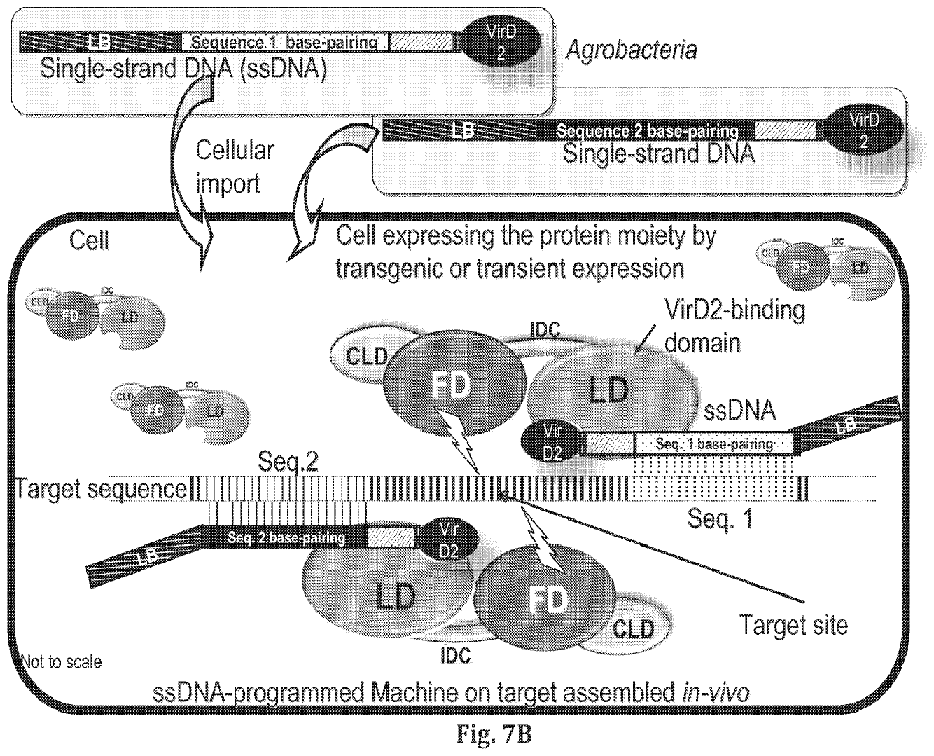

Reference is now made to FIGS. 7A-B which are schematic drawings (not to scale) showing non-limiting examples of delivery of the SCNA to a cell using a single-strand DNA produced in Agrobacterium. Shown in FIG. 7A is a non-limiting example of the use of Agrobacterium for production of ssDNA SCNA bound to a protein, VirD2, in vivo, at its 5′ end. As shown in this example, the targeting variable SCNA sequence is inserted into a multiple cloning site (MCS) in a plasmid capable of replicating in Agrobacterium. Agrobacterium is then transformed with this plasmid. The Ti plasmid Right Border (RB) sequence on the plasmid is cleaved and ssDNA is bound by VirD2 in the bacterium. 3 nucleotides of the RB sequence are left behind at the 5′ of the sequence after cleavage, and 21 nucleotides of the Ti plasmid left Border (LB) sequence are left behind after cleavage at the 3′ of the sequence. The LB sequence can further aid in SCNA stabilization and in screening for unwanted integration events. Mutated forms of Agrobacteria, (for example, those missing VirE1 or VirE2 or with partial VirD2 functionality) are useful for the inhibition of unwanted integration events. Agrobacterium then exports the T-DNA comprising the SCNA bound to VirD2 into the cell. Shown in FIG. 7B is a non-limiting example of the use of a bacterial secretion system to deliver SCNAs to a host cell. One or a multiplicity of agrobacteria transformed with different T-DNAs encoding different SCNA sequences are used to infect one cell. The VirD2-bound ssDNA SCNA thus created in the bacteria and exported to the host cell can then encounter and bind the Linking Domain of the protein moiety through an interaction between the VirD2 protein and the VirD2-binding domain in the Linking Domain in the host cell. An example for such a VirD2-binding Linking Domain comprises an artificial single-chain variable fragment of an antibody (scFv) produced against VirD2. The SCNA can thus bring about the assembly of the molecular complex on a Target nucleic acid.

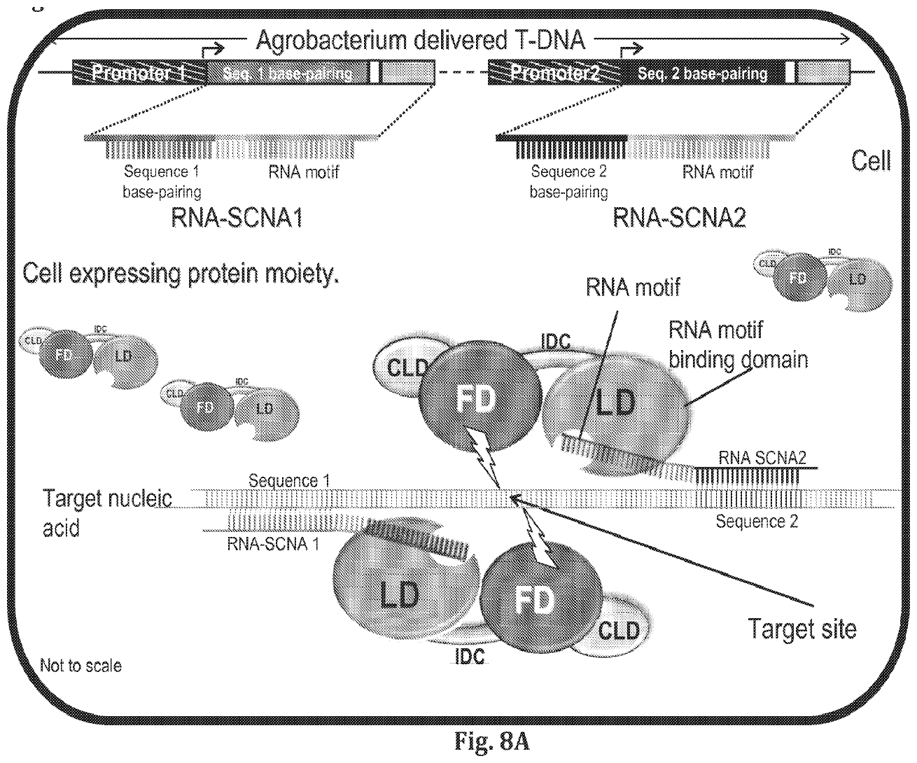

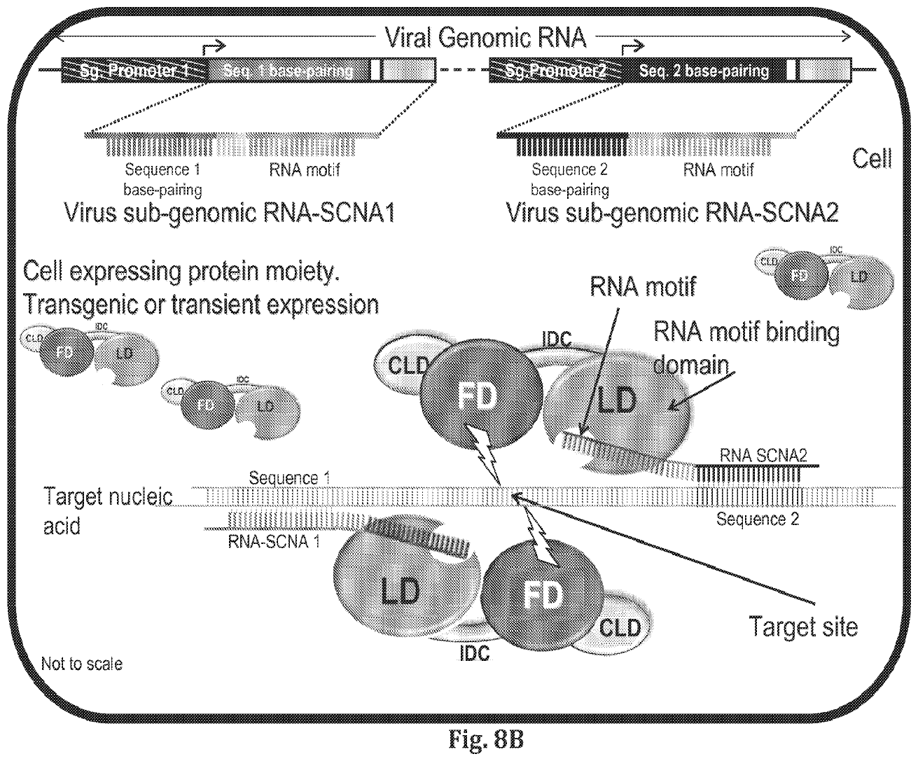

Reference is now made to FIGS. 8A-B which are schematic illustrations demonstrating the delivery of SCNA to a cell using RNA SCNAs produced inside the host cell, from an Agrobacterium delivered T-DNA (FIG. 8A) or from a nucleic acid delivered by an autonomously replicating vector such as a virus (FIG. 8B). The RNA SCNAs presented in these figures include an SCNA-RNA motif (marked combs) which can bind a corresponding RNA-binding motif of the Linking Domain of the protein moiety. As shown in FIG. 8A, the SCNA sequences are inserted into a multiple cloning site (MCS) in a plasmid capable of replicating in Agrobacterium and containing the appropriate Eukaryotic promoters for the transcription of one or a multiplicity of RNA SCNAs in the infected cell. FIG. 8B: The SCNA sequence(s) is/are inserted into the genome of a virus or a virus-derived autonomously replicating vector each under the control of a sub-genomic (sg) promoter for the transcription of one or a multiplicity of RNA SCNAs in the infected cell. In the non-limiting examples shown in FIGS. 8A-B, the nucleic acid molecule encoding for the protein-moiety coding may be delivered to the target cell beforehand, together with (concomitantly) or after the delivery of the SCNA encoding nucleic acid molecule. When the protein moiety and the SCNA are expressed in the cell, the assembly of the molecular complex on the target nucleic acid occurs.

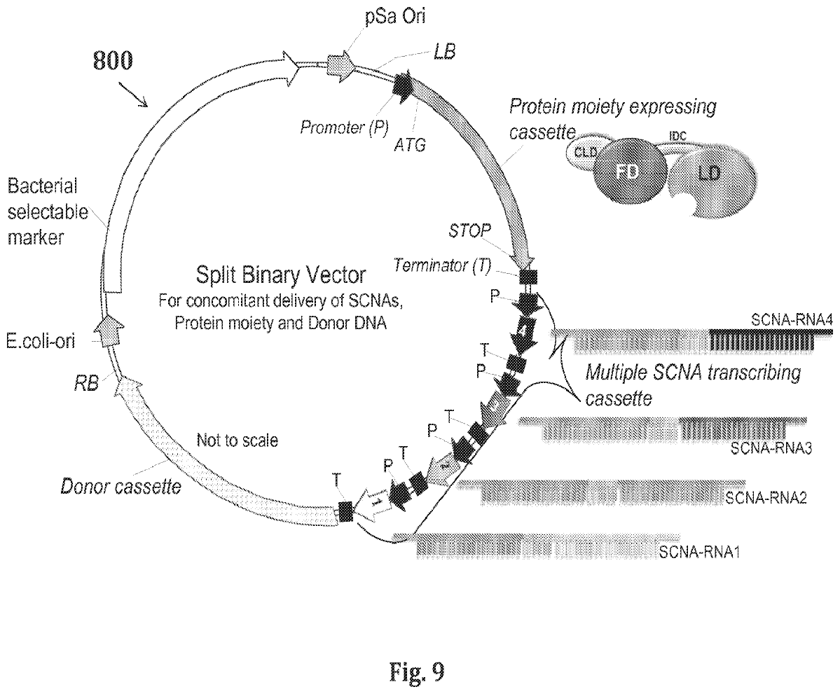

Reference is now made to FIG. 9, which shows a schematic illustration (not to scale) of a non-limiting example of a delivery vehicle or vector for concomitant delivery of the composition comprising the components necessary for the assembly of a programmable molecular complex to a susceptible target Eukaryotic cell in a single delivery event, according to some embodiments. For the non-limiting example shown in FIG. 9, the desired action is replacement of a genomic DNA stretch (the target nucleic acid), with a predetermined sequence the “Donor cassette”. Accordingly, the domains of the protein moiety include: a Functional Domain, derived from a nuclease and having a nucleic cleavage activity; a cellular localization domain, which is a nuclear localization signal (NLS); and Linking Domain capable of recognizing and binding an RNA motif on the SCNAs. In the example shown in FIG. 9, a biological delivery system is used. Agrobacterium is transformed with a plasmid vector, such as plasmid (800), which contains various functional/structural sequences, such as, bacterial selectable marker, various origins of replication sites (E. Coli-or, pSa Ori), LB sequence, promoter regions (designated as (P)), the protein moiety expressing sequence (comprising an ATG start codon and an in-frame STOP codon), Terminator site (T), multiple SCNA transcribing cassettes (shown as four SCNA transcribing cassettes, each comprising a promoter and terminator sequences), a Donor cassette, and RB site. The plasmid vector (transfected Agrobacterium) is then brought into contact with the target organism cells. Agrobacterium then forms a T-DNA from the region between the Right border (RB) and the Left border (LB) sequences and secretes it into the Eukaryotic cell. The ssDNA of the T-DNA is delivered to the nucleus, complemented in-vivo to become dsDNA, and transcribed to RNA from the compatible promoters (P) on the plasmid. The thus formed transcript of the protein moiety is translated to form the designated protein. Transcripts from the SCNA cassette which comprise an RNA motif sequence are bound by a specific RNA sequence-binding domain in the protein moiety. The Donor cassette contains a sufficiently long sequence that can recombine with the target nucleic acid in the presence of a double strand break (DSB) formed adjacent to the recombination site. The SCNAs are designed to target and hybridize sequences flanking the sequence to be replaced. In some embodiments, a similar plasmid, lacking border sequences, or a linear DNA of similar construction, can further be used to transfect cells in a non-biological delivery system, to the same effect.

According to some embodiments, and as detailed above, alterations/modifications in the targeted sequence include, for example, but not limited to: permanent deletion, mutation, insertion of nucleic acids, and replacement of a targeted sequence with another nucleic acid sequence, knocking-out, frame-shifting, or any change in any fashion of the transcription or translation of a gene, its regulatory sequences, the genes regulating the gene of interest or their regulatory sequences in a regulatory chain of events.

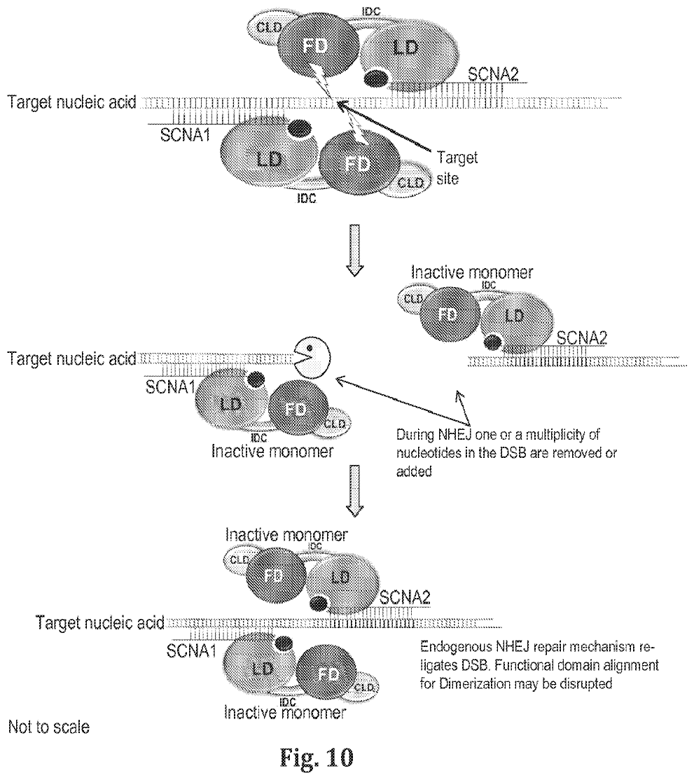

Reference is now made to FIG. 10, which is a schematic illustration (not to scale) demonstrating the use of a programmed molecular complex to create a mutation in a Target nucleic acid, according to some embodiments. As shown in the non limiting example presented in FIG. 10, the Functional Domain of the protein moiety is derived from a nuclease, and the mutation of the target site on the target nucleic acid is achieved through the creation of a dsDNA break (DSB) in the Target nucleic acid in a predefined location. The SCNA-programmed molecular complexes self-assemble by SCNA base-pairing with a corresponding target sequence on the target nucleic acid. Upon assembly of the components of the complex, the Functional Domain is dimerized and the nuclease is activated, cleaving the target site, which is located, in this example, at or near the midpoint, between the two SCNA molecules, thereby creating a DSB (for example, the DSB can have 4 nucleotide 5′-overhangs such as those created by the restriction enzyme FokI). Cellular Non homologous end-joining (NHEJ) repair mechanisms attempt to repair the DSB and while doing so may: 1) make a perfect ligation—while the complex may continue to recleave the same sequence for repeated attempts at mutation until depletion of complex components, 2) add one or a multiplicity of nucleotides thus widening the distance between the SCNAs and abolishing Functional Domain dimerization, thereby ending the action of the complex, or 3) remove one or a multiplicity of nucleotides (“pacman” figure), thus narrowing the distance between the SCNAs and abolishing the Functional Domain dimerization, thereby ending the action of the complex. When any of options 2 or 3 occur within the cell, a mutation is achieved.

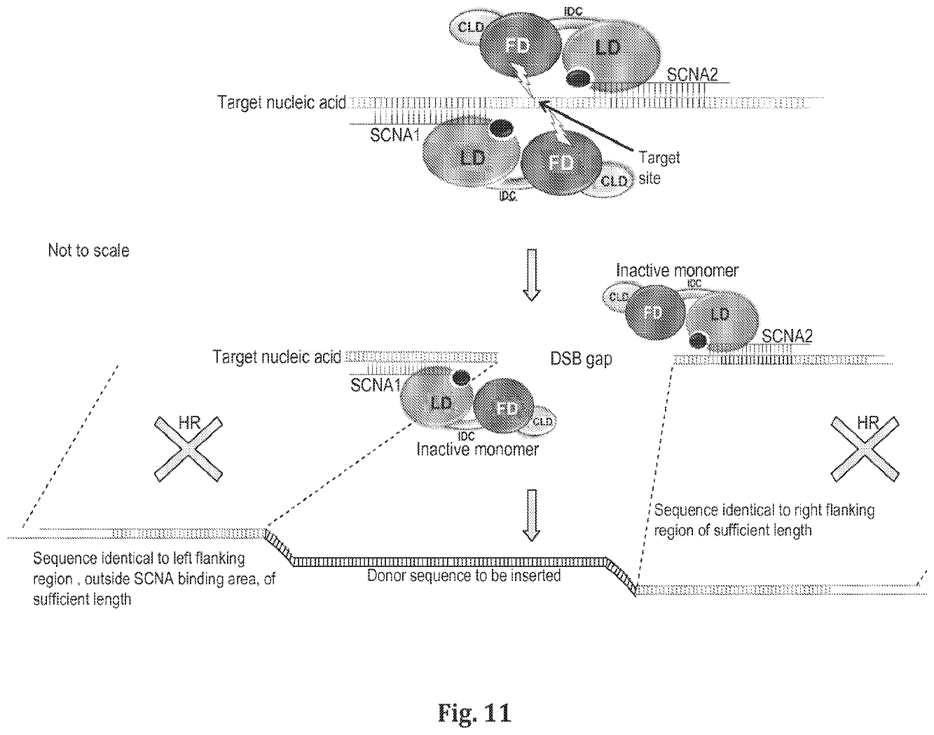

Reference is now made to FIG. 11, which is a schematic illustration (not to scale) demonstrating the use of a programmed molecular complex to insert one or a multiplicity of nucleotides into a Target nucleic acid using a supplied Donor nucleic acid, according to some embodiments. As shown in the non limiting example presented in FIG. 11, the Functional Domain of the protein moiety is derived from a nuclease, and a dsDNA break (DSB) in the target nucleic acid at a predefined location (target site) assists the process of Homologous Recombination (HR). The SCNA-programmed molecular complexes self-assemble by SCNA base-pairing with a corresponding target sequence. Upon assembly of the components of the complex, the Functional Domain is dimerized and the nuclease is activated, thereby cleaving the target nucleic acid at the target site, which may be located, for example, at or near the midpoint between the two SCNA molecules, thereby creating a DSB. The Donor DNA contains the sequence to be inserted and sufficiently long stretches of nucleotides, flanking this sequence which are essentially identical to the Target sequence flanking the intended DSB point. These flanking sequences may then recombine (X) with the target nucleic acid through the cellular process of HR, thus replacing a predetermined stretch of nucleotides in the Target nucleic acid, and in effect bringing about an Insertion of the desired sequence. Upon recombination and Insertion of the predetermined Donor sequence, the distance between the SCNAs is widened, thus interfering with dimerization of the Functional Domain, thereby ending the action of the complex. In occasions when perfect re-ligation by NHEJ occurs, the activated programmed complex may continue to recleave the same sequence for repeated attempts at insertion.

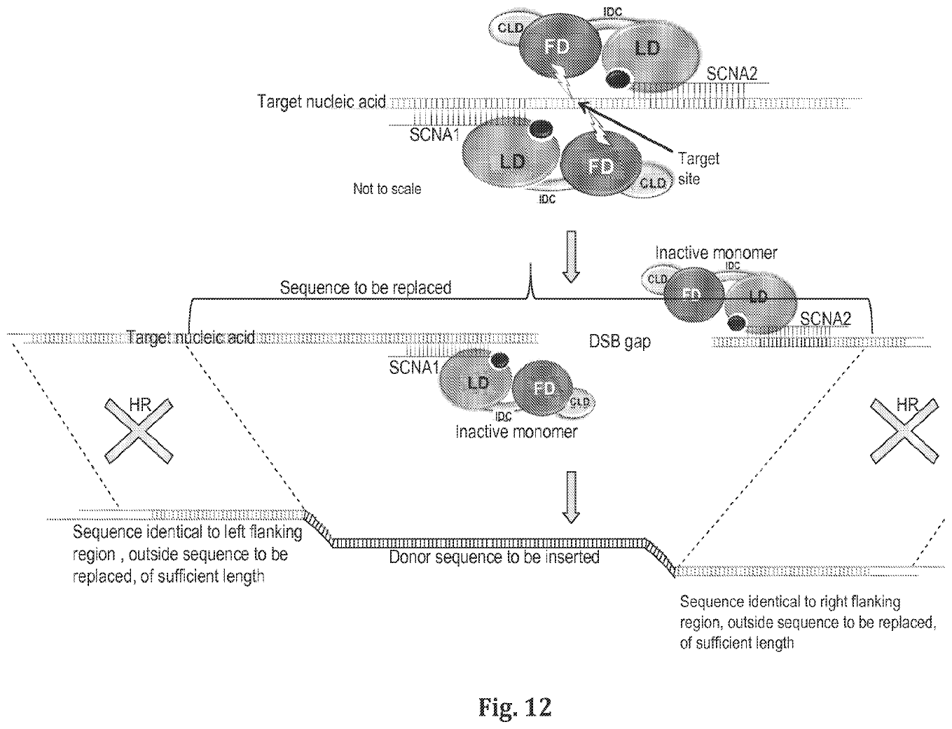

Reference is now made to FIG. 12, which is a schematic illustration (not to scale) demonstrating the use of a programmed molecular complex in the replacement, insertion and/or deletion of one or a multiplicity of nucleotides in a Target nucleic acid using a supplied Donor nucleic acid, according to some embodiments. As shown in the non limiting example presented in FIG. 12, the Functional Domain of the protein moiety is derived from a nuclease, and a dsDNA break (DSB) in the Target nucleic acid in a predefined location (target site) assists the process of Homologous Recombination (HR). SCNA-programmed molecular complexes self-assemble by SCNA base-pairing with a predetermined target sequence. Upon assembly of the components of the complex, the Functional Domain is dimerized and the nuclease is activated, cleaving the target nucleic acid at the target site, which may be located, for example, at or near the midpoint between the two SCNA molecules, thereby creating a DSB. The Donor DNA contains the exogenous sequence to be inserted instead of the endogenous target sequence to be removed as well as sufficiently long stretches of nucleotide flanking this exogenous sequence, which are essentially identical to the Target sequence flanking the intended sequence to be removed. These flanking sequences may then recombine (X) with the Target DNA through the cellular process of HR, thus replacing a stretch of DNA in the Target DNA and in effect bringing about a replacement of an undesired endogenous sequence by a desired exogenous sequence. Upon successful recombination and replacement of the desired exogenous sequence, the SCNA binding sites on the Target nucleic acid may be designed to be abolished, thus ending the action of the complex. In occasions when perfect re-ligation by NHEJ occurs, the complex may continue to recleave the same sequence for repeated attempts at recombination.

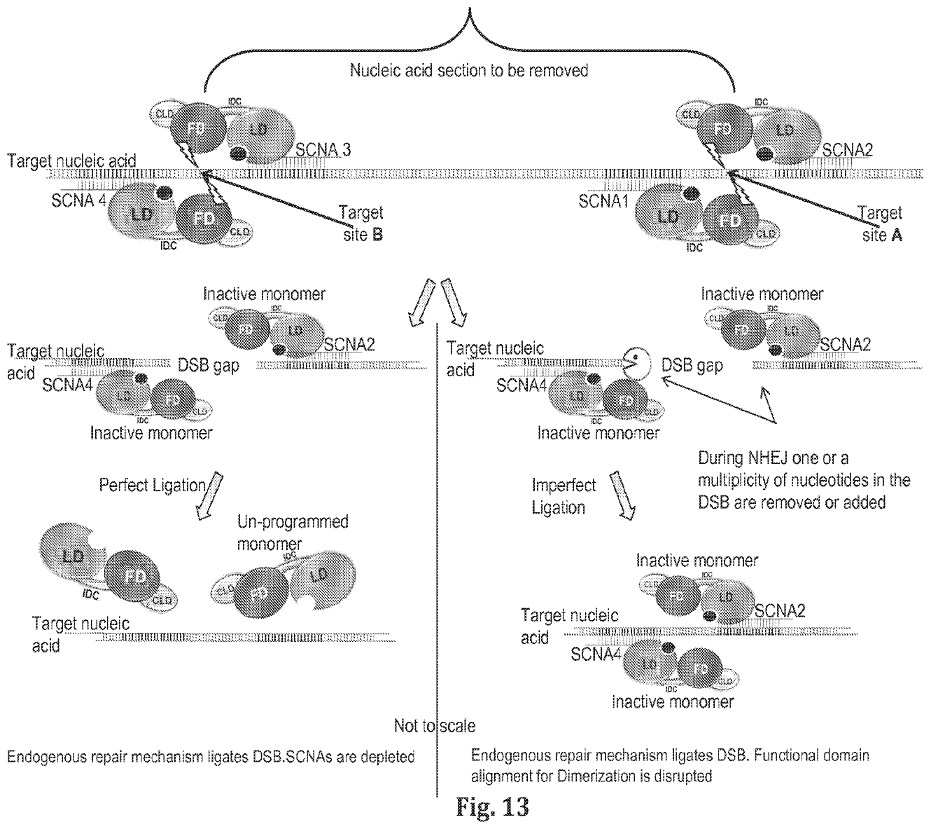

Reference is now made to FIG. 13, which is a schematic illustration (not to scale) demonstrating the use of a programmed molecular complex to create a deletion of one or a consecutive multiplicity of nucleotides from a target nucleic acid, according to some embodiments. As shown in the non limiting example presented in FIG. 13, the Functional Domain of the protein moiety is derived from a nuclease, and the deletion is achieved through the creation of two dsDNA breaks (DSBs) in the Target nucleic acid at two predefined locations. SCNA-programmed molecular complexes self-assemble by SCNA base-pairing with corresponding target sequences. Upon assembly of the components of the complex, the Functional Domains are dimerized and the nucleases are activated, cleaving the target nucleic acid at the target site, which may be located at or near the midpoint between each pair of SCNA molecules creating DSBs. Concomitant or sequential cleavage of both sites essentially eliminates, or deletes, the sequence in between. Cellular Non homologous end-joining (NHEJ) repair mechanisms attempt to repair the DSB and while doing so may: 1) make a perfect ligation of the target DNA flanking the deleted sequence, while the activate complex may continue to recleave the same sequence until depletion of complex components (left hand panel); 2) make a perfect re-ligation of each separate DSB—while the complex may continue to recleave the same sequence for repeated attempts at deletion until depletion of complex components; 3) remove one or a multiplicity of nucleotides (“pacman” figure, right hand panel) in the DSB gap, thus narrowing the distance between the SCNAs and abolishing Functional Domain dimerization, thereby ending the action of the complex; or 4) add one or a multiplicity of nucleotides in the DSB gap thus widening the distance between the SCNAs and abolishing Functional Domain dimerization, thereby ending the action of the complex.

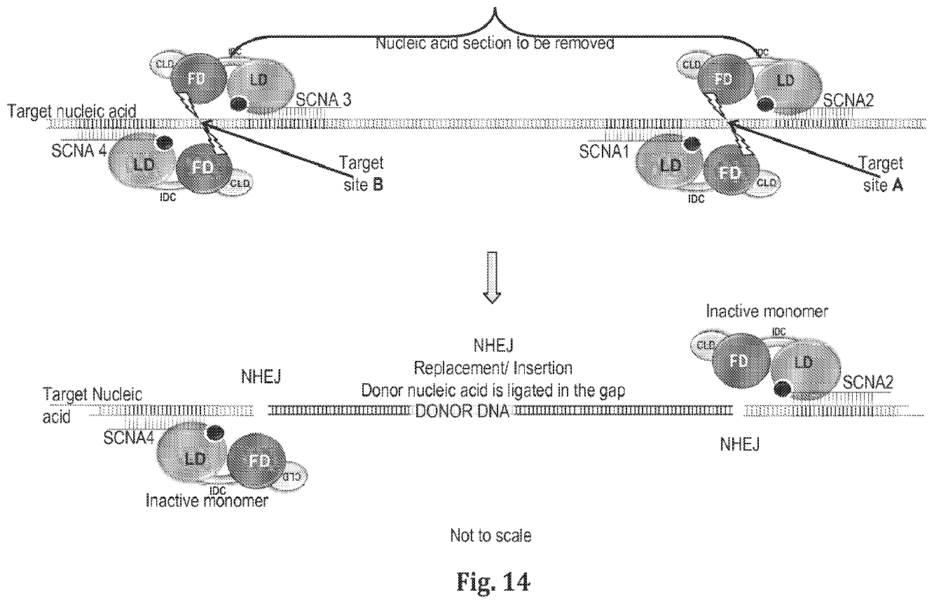

Reference is now made to FIG. 14, which is a schematic illustration demonstrating the use of a programmed molecular complex to replace one or a multiplicity of nucleotides in a Target nucleic acid using a supplied Donor nucleic acid, according to some embodiments. As shown in the non limiting example presented in FIG. 13, the Functional Domain of the protein moiety is derived from a nuclease, and the replacement is achieved through the creation of two dsDNA breaks (DSBs) in the Target nucleic acid in two predefined locations (target sites), creating a deletion, and supplying a linear or linearized DNA Donor to fill the gap. SCNA-programmed molecular complexes self-assemble by SCNA base-pairing with corresponding target sequences. Upon assembly of the components of the complex, the Functional Domains are dimerized and the nucleases are activated, cleaving the target at or near the midpoint between each pair of SCNA molecules, thereby creating DSBs. Concomitant or sequential cleavage of both sites essentially eliminates, or deletes, the sequence region in between. Cellular Non homologous end-joining (NHEJ) repair mechanisms attempt to repair the DSB and while doing so may: 1) make a perfect pair of ligations of the Donor into the Target abolishing Functional Domain dimerization, thereby ending the action of the complex; 2) make a perfect ligation of the target nucleic acid sequence flanking the deleted sequence—whereas the complex may continue to recleave the same sequence for repeated attempts at replacement until depletion of complex components; 3) make a perfect re-ligation of each separate DSB, whereas the complex may continue to recleave the same sequence for repeated attempts at replacement until depletion of complex components; 4) remove one or a multiplicity of nucleotides in a DSB gap, thus narrowing the distance between the SCNAs and abolishing Functional Domain dimerization, thereby ending the action of the complex; or 5) add one or a multiplicity of nucleotides in a DSB gap thus widening the distance between the SCNAs and abolishing Functional Domain dimerization thereby ending the action of the complex.

### Genetic Diseases

According to some embodiment, the compositions and methods of the present invention can be used to replace any genomic sequence with a homologous, non-identical sequence. For example, a mutant genomic sequence can be replaced by its wild-type counterpart, thereby providing methods for treatment of e.g., genetic disease, inherited disorders, cancer, and autoimmune disease. In like fashion, one allele of a gene can be replaced by a different allele using the methods disclosed herein. Exemplary genetic diseases include, but are not limited to, achondroplasia, achromatopsia, acid maltase deficiency, acquired immunodeficiencies, adenosine deaminase deficiency (OMIM No. 102700), adrenoleukodystrophy, aicardi syndrome, alpha-I antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum's disease, ectodermal dysplasia, Fanconi's anemia, fibrodysplasia ossificans progressive, fragile X syndrome, galactosemis, Gaucher's disease, generalized gangliosidoses (e.g., GM1), hemochromatosis, hemoglobinopathies (e.g., sickle cell anemia, the hemoglobin C mutation in the 6.sup.th codon of beta-globin, alpha-thalassemia, beta-thalassemia), hemophilia, Huntington's disease, Hurler Syndrome, hypophosphatasia, Klinefleter syndrome, Krabbes Disease, Langer-Giedion Syndrome, leukocyte adhesion deficiency (LAD, OMIM No. 116920), leukodystrophy, long QT syndrome, lysosomal storage diseases (e.g., Gaucher's disease, GM1, Fabry disease and Tay-Sachs disease), Marfan syndrome, Moebius syndrome, mucopolysaccahidosis (e.g. Hunter's disease, Hurler's disease), nail patella syndrome, nephrogenic diabetes insipdius, neurofibromatosis, Neimann-Pick disease, osteogenesis imperfecta, porphyria, Prader-Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner's syndrome, urea cycle disorder, von Hippel-Landau disease, Waardenburg syndrome, Williams syndrome, Wilson's disease, Wiskott-Aldrich syndrome, X-linked lymphoproliferative syndrome (XLP, OMIM No. 308240).

The following examples are presented in order to more fully illustrate some embodiments of the invention. They should, in no way be construed, however, as limiting the broad scope of the invention.

### EXAMPLES

### Example 1—In-Vivo System as Bioassay for Tuning Components of the Molecular Complex

This example describes a bioassay suitable for testing and optimizing permutations in the design and use of the programmable molecular complex, such as, for testing its activity in different organisms or cells, for testing different delivery methods, and for testing the editing functions of mutation, replacement, deletion and insertion.

The experiments shown in the examples below are for the detection of gene targeting and specific cleavage by a composition of the programmable molecular complex, which includes a modified nuclease as the effector domain of the protein moiety.

Visual reporter systems are used, based on repair of a STOP codon which is placed inside the reporter coding sequence. The reporter in these examples is Green Fluorescent Protein (GFP). When targeted, a double strand breaks (DSB) formed by the activated complex is repaired, (presumably through NHEJ pathway as exemplary illustrated in FIG. 10), abolishing the STOP codon and restoring GFP activity. This assay may thus give a good indication of gene targeting efficiency. This assay is known also as the “STOP GFP” assay. This visual assay is designed to target plasmid or genomic DNA in-vivo. In the following examples, an Arabidopsis protoplast based bioassay is used. In the described bioassay, the aforementioned reporter systems are delivered into protoplasts on a plasmid, co-delivered with the plasmid expressing the protein moiety of the molecular complex in-vivo and co-delivered with a pair of ssDNA Specificity Conferring Nucleic Acids (SCNA) modified, in this example, with a terminal (NHS-Ester-)-Digoxigenin (DIG). A second modification for exonuclease protection, (phosphorothioate), is added at the opposite terminus (here marked with an asterisk). The plasmid vectors used herein comprise plant promoters.

### Protein Sequence and Properties

The molecular complex designed for this application is composed of two sequences of homologous nucleic acids for specificity determination (SCNAs) and a chimeric protein component containing a nuclease which binds to the SCNAs in vivo. The resulting cleavage of the predetermined target site (STOP codon) of the target nucleic acid (GFP coding sequence) results in its desired mutation, by endogenous processes. The programmable molecular complex in this example consists of 2 identical monomers of a protein moiety and two different SCNA molecules (as schematically illustrated in FIGS. 1A and 2A). In this example the protein moiety contains an amino-acid sequence modified from a FokI nuclease domain as the Functional Domain; an amino-acid sequence adapted from anti-DIG (Digoxigenin) single-chain variable fragment (scFv) immunoglobin (DIG-ScFv) similar to that described in (Huston et. al, 1988) as Linking Domain; an SV40NLS (SEQ ID NO: 3, PKKKRKV) as a nuclear localization domain and a ˜15 Å inter-domain connector (SEQ ID NO:7, GGSGG). The nucleic acid sequence encoding for the protein moiety is inserted into suitable expression vectors (pUC based vectors (pSAT)), including a NOS or 35S promoter.

The in-vivo binding between the specificity-conferring nucleic acid and the Linking domain of the protein moiety, in this example, is the result of a non-covalent interaction which can be described as an antibody-antigen interaction; single-chain antibody-antigen; antibody or single-chain antibody-hapten interaction.

In this example, the nucleic acid end-modification of the SCNA is an NHS-Ester linked Digoxigenin (DIG) that is attached to the 5′ or 3′ position of the SCNA oligonucleotide.

Amino-acid sequence (one letter code) of the protein moiety of the molecular complex (NLS-FokI-nuclease sequence With Digoxygenin ScFv) is as designated in SEQ ID NO: 12, and is encoded by the sequence as set forth in SEQ ID NO: 13.

### SCNA Properties and Sequence

The length of the SCNA of the complementary, target-base-pairing oligonucleotide is preferentially at least 18 bases. The SCNA can also contain a small number (e.g. 1-6, in one example 6, in other example, 2) of non-target-base-pairing nucleotides (N's) of any sequence composition that serve as a spacer between the DIG-NHS terminal-modifier and the target-complementary nucleotides. As detailed above, due to histones occupying minor grooves of DNA in chromosomal DNA, some constraints on SCNA spacing may exist. Thus, SCNAs are preferably designed to fit in the target DNA major groove by modulating the distance between SCNAs, to enable an orientation of the target helix that allows Linking Domains of a dimerized programmable molecular complex to bind. Choice of interdomain connector between the globular Functional Domain and the Linking Domain (in the example shown here is GSLEGGSGG (SEQ ID NO: 14)) also influences the optimal SCNA distance as it either restricts or permits movement in the “hinge” between these two domains. Addition of non-target-base-pairing nucleotides (“N's”) changes both the distance between SCNAs and the rotational orientation on the target helix as it changes the flexibility of the SCNA relative to the protein and the helix. These unpaired nucleotides are not constrained to the target DNA major groove.

The results of spatial measurements taken from computerized 3D models for the anti-DIG-ScFv-NHS-Ester-DIG system with the GSLEGGSGG (SEQ ID NO: 14) interdomain linker, as shown in this example, yielded that the expected optimal distance between SCNAs is, in the presence of 2 N's in the SCNA, about 23-26 nucleotides. Cleavage is predicted to occur about +2 nucleotides to left and to the right of the 11^{th}, 12^{th} or 13^{th} nucleotide, counting from after the last nucleotide hybridizing with the SCNA on either side, taking into account the 4 base 5′ overhang created by dsDNA cleavage by the dimerized construct. This criterion suggests that if the targeted sequence is, for this 24 nucleotide example: AAAAAAAAAAYYYYYYYYYXXXXXXYYYYYYYYYCCCCCCCCCC, where Y+X represents the number of nucleotides between the SCNA base-pairing sites, then the designed SCNAs base-pair with areas A and C and the cleavage resulting in DSB is in or adjacent to the X area. The SCNAs can be complementary to either sense or antisense strands, but are chosen preferably to base-pair with the sense (untranscribed) sequence. Both SCNAs can base-pair with the same strand, as the protein moiety's position is situated at the “near end” of the SCNA as defined by the 5′ or 3′ modification of the primer being at the “near end” (as illustrated in FIG. 2A). Distance optimization between SCNAs, as well as preferred strand, are one of several criteria tested in this bioassay.

Target nucleic acid (GFP coding sequence), containing a target site (STOP codon, (TAG)) includes the nucleotide sequence set forth in SEQ ID NO: 15 (“STOP-GFP”), where the TAG stop codon is located at nucleotide 878:

The mCherry donor for examples 1B and 1C includes a promoter-less and terminator-less coding sequence, set forth in SEQ ID NO: 16:

The following target site sequence is targeted in examples 1A through 1C:

### Examples 1A-C “First Target” Sequence

(SEQ ID NO: 17)

GTCGACAACTAGTCCAGATCT

### SCNA Sequences

Modification symbols are: Phosphorothioate-bonds=*; 5′ DIG=/5DigN/; 3′DIG=/3DigN/).

### Tested Paired SCNA Combinations for 1A-1C “First Target”:

Sense SCNA:
GFP_918_SR1:
/5DigN/NNNNNNGTGTCCAAGGGCGAGGAGCTG*T;
(the nucleic acids only are designated herein
as SEQ ID NO: 18)
GFP_896_SL1:
T*TTACGAACGATAGCCATGGCCNNNNNN/3DigN/
(the nucleic acids only are designated herein
as SEQ ID NO: 19)

A second Sense paired combination, employing a 24 bp target gap and a shorter SCNA linker according to the prediction results:

GFP_920_SR1:
/5DigN/NNGTCCAAGGGCGAGGAGCTGTT*C
(the nucleic acids only are designated herein as
SEQ ID NO: 20)
GFP_895_SL1:
A*TTTACGAACGATAGCCATGGCNN/3DigN/
(the nucleic acids only are designated herein as
SEQ ID NO: 21)
Anti-sense SCNA:
GFP_918_ASR1:
C*AGCTCCTCGCCCTTGGAGACNNNNNN/3DIGN/
(the nucleic acids only are designated herein as
SEQ ID NO: 22)
GFP_896_ASL1:
/5DIGN/NNNNNNGGCCATGGCTATCGTTCGTA*A
(the nucleic acids only are designated herein as
SEQ ID NO: 23)

A second Anti-sense paired combination, employing a 24 bp target gap and a shorter SCNA linker according to the prediction results:

GFP_920_ASR1:
G*AACAGCTCCTCGCCCTTGGACNN/3DIGN/
(the nucleic acids only are designated herein as
SEQ ID NO: 24)
GFP_895_ASL1:
/5DIGN/NNGCCATGGCTATCGTTCGTAAA*T
(the nucleic acids only are designated herein as
SEQ ID NO: 25)

Combinations of sense and anti-sense pairs:

GFP_918_SR1:
/5DigN/NNNNNNGTGTCCAAGGGCGAGGAGCTG*T
(the nucleic acids only are designated herein as
SEQ ID NO: 18)
GFP_896_ASL1:
/5DIGN/NNNNNNGGCCATGGCTATCGTTCGTA*A
(the nucleic acids only are designated herein as
SEQ ID NO: 23)

A second Anti-sense paired combination, employing a 24 bp target gap and a shorter SCNA linker according to the prediction results:

GFP_920_SL1:
A*TTTACGAACGATAGCCATGGCNN/3DigN/
(the nucleic acids only are designated herein as
SEQ ID NO: 21)
GFP_895 ASR1:
G*AACAGCTCCTCGCCCTTGGACNN/3DIGN/
(the nucleic acids only are designated herein as
SEQ ID NO: 24)
“First target” for example 1C is identical to the
1A and 1B target
“Second target” for example 1C:
(SEQ ID NO: 26)
GACTCTAAGCTTGGGTCTAGA

A second Anti-sense paired combination, employing a 24 bp target gap and a shorter SCNA linker according to the prediction results:

GFP_920_SR1:
/5DigN/NNGTCCAAGGGCGAGGAGCTGTT*C/
(the nucleic acids only are designated herein as
SEQ ID NO: 20)
GFP_895_ASL1:
/5DIGN/NNGCCATGGCTATCGTTCGTAAA*T
(the nucleic acids only are designated herein as
SEQ ID NO: 25)
GFP_918_ASR1:
C*AGCTCCTCGCCCTTGGAGACNNNNNN/3DIGN/
(the nucleic acids only are designated herein as
SEQ ID NO: 22)
GFP_896_SL1:
T*TTACGAACGATAGCCATGGCCNNNNNN/3DigN/
(the nucleic acids only are designated herein as
SEQ ID NO: 19)

### SCNAs for Example 1C:

A combination, utilizing a 24 bp target gap and a short SCNA linker:

Sense:
GFP_1658_SR:
/5DIGN/NNTCCGCAAAAATCACCAGTCTC*T
(the nucleic acids only are designated herein as
SEQ ID NO: 27)
GFP_1633_SL:
G*CATGGACGAGCTGTACAAGTCNN/3DIGN/
(the nucleic acids only are designated herein as
SEQ ID NO: 28)
Antisense:
GFP_1658_ASR:
A*GAGACTGGTGATTTTTGCGGANN/3DIGN/
(the nucleic acids only are designated herein as
SEQ ID NO: 29)
GFP_1633_ASL:
/5DIGN/NNGACTTGTACAGCTCGTCCATG*C
(the nucleic acids only are designated herein as
SEQ ID NO: 30)

As in example 1A-C “first target” SCNAs these four example 1C “second target” SCNAs may be paired using one “left” (L) and one “right” (R) SCNA from the list above.

### Delivery

Bioassay setup: Arabidopsis protoplast preparation is based on Wu et. al. (Wu et. al., 2009):
Plant material: Arabidopsis grown under 16 hr day optimal light (150 microEinstein·m−2·s−1) at 22 degrees C.
Leaves: 3-5 week old plants (W˜2 cm L˜5 cm).

### Working Solutions:

Enzyme solution: 1% Cellulase, 0.25% Macerozyme, 0.4M Mannitol, 10 mM CaCl₂), 20 mM KCl, 0.1% BSA, 20 mM MES pH5.7. Heat 50-55 degrees C. 10 minutes to inactivate proteases and then filter. Use fresh. 10 ml/7-10 peeled leaves (1-5 gr)/dish.
Modified W5 solution: 154 mM NaCl, 125 mM CaCl₂), 5 mM KCl, 5 mM Glucose, 2 mM MES pH5.7. Wash twice with 25 ml/plate, +twice 3 ml for transfection wash+1 ml resuspension
Modified MMg solution: (Resuspension solution) 0.4M Mannitol, 15 mM MgCl2, 4 mM MES pH5.7.
Modified TEAMP transfection buffer (PEG solution): 40% PEG MW 4000, 0.1M CaCl₂), 0.2M Mannitol volume=1:1 of 200 microliter protoplasts in MMg+volume of DNA

### BSA: 1% BSA

### Working Protocol:

1. Preheat waterbath to 50-55 degrees C., cool swing-out centrifuge, chill W5 and MMg, and cut tips.
2. Prepare fresh BSA coated plates (1.25 ml 1% BSA/well in water, incubate on bench till ready)
3. Make fresh enzyme solution 1 ml/treatment.
4. Pick 7-10 leaves, must not be wet. 10 leaves should yield ˜4-5 transformations.
5. Tape upper epidermis with Time-tape, lower with Magic tape. Easier without gloves. Easier to peel if petiole is stuck to time-tape only.
6. 0.22 μm-filter 10 ml fresh enzyme solution into each petri dish
7. Peel and discard Magic tape. Transfer Time-tape side to petri dish
8. Gently shake on platform shaker 40 rpm 20-60 min in light until protoplast release (check empirically)
9. Centrifuge in 50 ml tubes 100×g 3 min in swing-out rotor
10. Wash twice with 25 ml cold W5 solution.
11. Ice 30 min, count during this time in hemocytometer using light microscope
12. Centrifuge and resuspend in MMg solution to 2-5×10\hat{ }5 cells/ml (about 1 ml).

### Transfection:

1. Make fresh PEG sol for transfection in 2 ml tube
2. Pour off BSA from 6-well plates and dry
3. Mix ˜5×10\hat{ }4 protoplasts (2×10\hat{ }4-1×10\hat{ }5) in 0.2 ml MMg solution with a mixture of Target plasmid DNA, Protein Moiety expressing plasmid DNA and SCNAs ssDNA to a total of 30-40 microgram at RT in 15 ml round-bottom (snap-cap) tubes.
4. Add equal volume (0.2 ml protoplasts+midiprep vol.) of fresh PEG sol
5. Incubate RT 5 min
6. Wash by slowly adding 3 ml W5 solution, 1 ml at a time, and mixing
7. Centrifuge 100×g in swing-out 1 min
8. Repeat wash and pellet
9. Resuspend in 1 ml W5 solution
10. Pour into BSA-coated plates
11. Grow protoplasts under 16 hr day optimal light (150 microEinstein·m\hat{ }−2·s\hat{ }−1) at 22 degrees C., replacing media as needed.
   Protoplasts suspended in W5 solution are screened for GFP/mCherry activity 3 days after transfection using an automated flow-cytometer (FACS). GFP is detected by excitation at 488 nm with emission detected by 530/30 filter. mCherry excitation and emission are 561 nm and 610/20 filter. Threshold and compensation factors are set to exclude any false positives.

### Example 1A: Point Mutation by Induced DSB

In this example, cleavage of the target results in a Double-Strand-Break (DSB) in the plasmid DNA target. This DSB is designed to be created in the STOP codon site, which is digested and is repaired by the NHEJ repair mechanism as set forth in the exemplary illustration of FIG. 10 (mutation). NHEJ is prone to mutations, and some of these mutations may abolish the STOP codon and restore an open reading frame resulting in an active GFP open reading frame (ORF). GFP is then detected by means of microscopy or flow cytometer (FACS), enabling the measurement of system efficiency and comparison between variables for its improvement.

When targeting a STOP-GFP transgene previously stably introduced into the Arabidopsis genome (instead of a plasmid), genome-modified plants can be regenerated from GFP expressing protoplasts.

### Example 1B: Specific Integration into an Induced Genomic DSB

Similar to the example 1A, the in-frame GFP stop codon sequence is targeted with the programmed molecular complex. In this application a linear dsDNA donor is added, comprising a promoter-less, terminator-less mCherry reporter gene containing only the CDS. Following transfection as described, mCherry expressing protoplasts are detected by red fluorescence by means of microscopy or flow cytometer (FACS), enabling the measurement of system efficiency and comparison between variables for its improvement. The mCherry excitation and emission are 561 nm and 610/20 filter. Since the donor DNA contains a promoter-less mCherry, its activity can be achieved by promoter trapping. Thus, the targeted GFP cassette is cleaved to form a DSB wherein any linear DNA may be ligated. Since excess of the mCherry CDS linear dsDNA is supplied, it is trapped in the DSB, causing, in some cases, translation in frame of the mCherry protein. Targeted plasmids with such specific insertion of the mCherry into the GFP targeted sequence are further analyzed by PCR with the following primers: one binding the target plasmid DNA sequence, and one binding the inserted DNA:

35SF:

(SEQ ID NO: 31)

CTATCCTTCGCAAGACCCTTCC
mCherryR:

(SEQ ID NO: 32)

TTATCTTGTACAGCTCGTCCAT

Similarly, a bacterial antibiotic resistance (NPT-II coding cassette, without an origin of replication) is provided into the protoplasts as a linear dsDNA. This DNA is inserted instead of the mCherry CDS of examples 1B and 1C, and screened by extracting total DNA from protoplasts, transforming the DNA including plasmids with or without insertions into E. coli, and growing these on a medium containing Kanamycin. Resistant bacteria have plasmids that trapped the NPT-II cassette. To assess the specificity of the insertion into the predetermined GFP target site, the GFP-target site is PCR-amplified with primers spanning the expected insertion site. Specific insertion causes a significant shift in size of the PCR product on an agarose gel. Efficiency of insertion is calculated by dividing the number of Kanamycin resistant colonies by the number of Ampicillin resistant colonies (Ampicillin resistance is encoded on the target plasmid) in a duplicate-plating experiment. Specificity is calculated by repeating the experiment omitting or replacing components of the programmable molecular complex (e.g. GFP-targeting SCNAs) and comparing to unmodified experiments.

### Example 1C: Gene Replacement Through NHEJ Repair Mechanism

In this example, the GFP coding sequence is replaced with mCherry CDS via endogenous NHEJ. To delete an extensive section of target DNA via the NHEJ strategy, two DSBs are created. To target the beginning and the end of the GFP CDS, two sets of SCNAs are used in conjunction with the mCherry linear dsDNA donor. Since the donor DNA contains promoter-less mCherry, its activity can be achieved by promoter trapping. The targeted GFP cassette can therefore trap the mCherry CDS. The mCherry is analyzed by FACS or microscope with excitation and emission detected at 561 nm and 610/20 filters, respectively.

mCherry positive protoplast are sorted by FACS and subsequently subjected to DNA extraction, direct transformation of the total DNA which includes plasmids into E. coli, growth on antibiotic containing media, and performing two colony-PCR reactions on each bacterial colony with two primer sets:

35SF:

(SEQ ID NO: 31)

CTATCCTTCGCAAGACCCTTCC
mCherryR:

(SEQ ID NO: 32)

TTATCTTGTACAGCTCGTCCAT
and
35S-T-R-SEQ:

(SEQ ID NO: 33)

CCCTATAAGAACCCTAATTCCC
mCherryF:

(SEQ ID NO: 34)

ATGGTGAGCAAGGGCGAGGA

Colonies which produce an amplification product in both PCR reactions contain a plasmid which has been targeted in Arabidopsis protoplasts to produce a correctly oriented replacement event through the NHEJ repair pathway, and are further sequenced for verification.

When targeting a GFP transgene previously stably introduced into the Arabidopsis genome (instead of a plasmid), no such direct transformation of E. coli is performed. Instead, genomic DNA is amplified directly by PCR from single protoplasts using said primers. Alternatively, genome-modified plants can be regenerated from non-GFP expressing, mCherry expressing protoplasts, portions of which can be similarly analyzed.

### Example 2. DNA Double Strand Break Induction, Mutation and Insertion in a Monocotyledon Cereal Plant Genome

### Targeting IPKL in Maize for Knockout.

The IPK1 gene, encodes inositol-1,3,4,5,6-pentakisphosphate 2-kinase which is involved in phytate biosynthesis in maize seeds. Phytate, when fed to non-ruminant livestock, is an anti-nutritional component that contributes to environmental phosphorus pollution. Targeting IPK1 may reduce the seed phosphorus by 75%. Two paralogous Zea mays IPK genes sharing 98% sequence identity exist in the maize genome. In this example, the IPK1 sequence based on Genbank Accession #: EF447274 is targeted.

### Target Site in the Target Nucleotide Sequence:

In IPK1 exon 2:

(SEQ ID NO: 35)

TTCTCAAGTCATGAGCAACTC

### Protein Sequence and Properties

The resulting cleavage of the predetermined Target site IPK1 by the programmed molecular complex, result in its mutation or in insertion of a donor DNA into the DSB created by the programmed complex, as desired, aided by endogenous processes. The programmable molecular complex here consists of 2 identical monomers of a protein moiety and two different SCNA molecules. In this example, the protein moiety is identical to that of example 1.

In this example the nucleic acid end-modification of the SCNA is an NHS-Ester linked Digoxigenin (DIG) that is attached to the 5′ or 3′ position of the oligonucleotide.

### SCNA Properties and Sequence

The rational design of the SCNA is essentially as described in Example 1. The length of the SCNA of the complementary, target-base-pairing oligonucleotide is preferentially at least 18 bases. The SCNA can also contain a small number (e.g. 1-6, in one example 6, in other example, 2) of non-target-base-pairing nucleotides (N's) of any sequence composition that serve as a spacer between the DIG-NHS terminal-modifier and the target-complementary nucleotides.

### SCNA Nucleotide Sequences Flanking the IPK1 Target Site

Combinations of the following “R” and “L” SCNAs employing a 21 bp target gap are tested:

IPK1-SR-1710:
/5DIGN/NNNNNNCTGTGGGGCCATATCCCAGAA*C
(the nucleic acids only are designated herein as
SEQ ID NO: 36)
IPK1-SL-1688:
G*CGGGCACCGAGTTGTATTGTANNNNNN/3DIGN/
(the nucleic acids only are designated herein as
SEQ ID NO: 37)
IPK1-ASR-1710:
G*TTCTGGGATATGGCCCCACAGNNNNNN/3DIGN/
(the nucleic acids only are designated herein as
SEQ ID NO: 38)
IPK1-ASL-1688:
/5DIGN/NNNNNNTACAATACAACTCGGTGCCCG*C
(the nucleic acids only are designated herein as
SEQ ID NO: 39)

A second set of paired “R” and “L” SCNAs combination, employing a 24 bp target gap and a shorter SCNA linker according to the prediction results:

IPK1-SR-1712:
/5DigN/NNGTGGGGCCATATCCCAGAAC*T
(the nucleic acids only are designated herein as
SEQ ID NO: 40)
IPK1-SL-1687:
A*GCGGGCACCGAGTTGTATTGTNN/3DigN/
(the nucleic acids only are designated herein as
SEQ ID NO: 41)
IPK1-ASL-1687:
/5DigN/NNACAATACAACTCGGTGCCCGC*T
(the nucleic acids only are designated herein as
SEQ ID NO: 42)
IPK1-ASR-1712:
A*GTTCTGGGATATGGCCCCACNN/3DigN/
(the nucleic acids only are designated herein as
SEQ ID NO: 43)
SCNAs comprise modified ssDNA. Modification symbols
are: Phosphorothioate-bonds = *;
5′ DIG = /5Dig/; 3′DIG = /3DigN/.

### Experiment 2A: IPK1 Knockout and GFP Expression in Protoplasts

In this experiment, genomic DSB in Maize plants and specific integration of GFP sequence into the IPK1 gene forming a knockout mutation and expression of GFP in the IPK1 locus are tested. The programmed molecular complex forms the genomic DSB in the IPK1 sequence, initiating the integration of the donor DNA into the IPK1 sequence through homologous recombination.

This example, 2A, is performed on maize protoplasts which are analyzed by FACS for GFP activity.

### Working Protocol:

### Protoplast Preparation:

A transient expression assay using maize mesophyll protoplasts (Sheen, 2001) is used with electroporation-induced nucleic-acid delivery in addition or alternatively to a Polybrene-induced delivery protocol:

### Transfection Based on (Antonelli & Stadler, 1989):

Freshly isolated protoplasts (about 2×10\hat{ }6) are incubated for about 6 to 12 h with about 20-50 microgram of transfecting DNA comprising modified-ssDNA SCNAs, a plasmid encoding the Protein Moiety, Donor DNA (where applicable), and 30 microgram of the polycation Polybrene (hexadimethrine bromide). At the end of the incubation period, the transfection mixture is diluted by addition of growth medium and the cells are then incubated further for about 30 h before being assayed for transient gene expression:
1. Prepare protoplasts, and resuspend 2×10\hat{ }6 cells in 0.5 ml Murashige Skoog-based growth medium with 8% mannitol (MS2D8M).
2. For each experiment, prepare a fresh Polybrene (Aldrich) stock solution (10 mg/ml in phosphate buffered saline, pH 7.0). This is an extremely hygroscopic chemical and the manufacturer's safety instructions must be rigorously applied. The stock solution is then diluted to yield a final concentration of 30 microgram Polybrene in 0.1 ml MS2D8M.
3. The desired concentration of transfecting DNA—plasmid DNA and modified ssDNA-SCNAs—is suspended in 0.4 ml MS2D8M.
4. Mix the 0.1 ml (30 microgram) Polybrene solution with the resuspended protoplasts and transfer to a 60 mm Petri dish.
5. Immediately add (dropwise) the 0.4 ml DNA suspension. The protoplast/Polybrene/DNA mixture (total volume 1.0 ml) is rotated gently (25 rpm) on a gyrotary shaker for 15 min and then incubated (stationary) at 28 C for 6 h.
6. After the 6 h incubation, dilute the above mixture with 4.0 ml MS2D8M, seal the Petri dish, and follow procedures for assaying transient gene expression or for selection of stable transfectants.

### Detection:

Transfected maize protoplasts suspended in MS2D8M solution are analyzed by flow-cytometer using Fluorescence-activated cell sorting (FACS), 3 days after transfection with Polybrene. GFP is detected by excitation at 488 nm with emission detected by 530/30 filter. Threshold and compensation factors are set to exclude any false positives. FACS is used to separate targeted cells for further analysis.

The protoplasts are subjected to analysis by extraction of genomic DNA and its amplification by PCR using the primers 1F and 1R below and subsequent digestion with BspHI of the PCR product. BspHI uncleavable products of more or less similar size to wild-type result from precise targeting events coupled with imprecise re-ligation, larger sized PCR-products result from insertions into the target site as desired.

Primer 1F:

(SEQ ID NO: 44)

GAGCTAGATAGCAGATGCAGAT
Primer 2R:

(SEQ ID NO: 45)

CTCCAGAAAATCCCTAGAAACA

Alternatively, the PCR product is subjected to CEL I Enzymatic Mutation Detection Assay, in accordance with the instructions in the SURVEYOR Mutation Detection Kit (Transgenomics, USA). This assay is used to evaluate the effectivity of mutation of IPK1 DNA by gene targeting by the programmed molecular complex.
Donor sequence for experiment 2 A: GFP is fused to IPK1 sequence and thus GFP expression can happen only by precise homologous recombination (HR). The sequence of the entire donor sequence is as set forth in SEQ ID NO: 46. The sequence homologous to IPK1 necessary for recombination is nucleotides 1-621 and 1960-2610 of SEQ ID NO:46, and the GFP cassette is encoded by nucleotides 622-1959.

### Experiment 2B: IPK Knockout and Bar Insertion, Delivery to Calli

In this experiment, genomic DSB in Maize plants and specific integration of the herbicide bar resistance gene conferring resistance to Bialaphos (Phosphinothricin; Glufosinate-Ammonium; its analogues or commercial herbicides such as Basta, Bayer Crop Science) into the IPK1 gene forming knockout mutation and expression of bar in the IPK1 locus, are tested. The programmed molecular complex forms the genomic DSB in the IPK1 sequence initiating the integration of the donor DNA into the IPK1 sequence through homologous recombination.

This example is performed on maize calli which are transfected by DNA bombardment and then grown under Bialaphos (Basta) selection.

### Working Protocol:

1. Formation of embryogenic callus: Immature embryos 1.6 mm to 1.8 mm (Plants A188XB73 or A188XB84) Growth conditions: Light 10 microEinstein/m\hat{ }2/sec 24 degrees C. on N6 medium containing 2 mg/L glycine, 2.9 g/L L-proline, 100 mg/L casein hydrolysate, 13.2 mg/L dicamba or 1 mg/L 2,4D, 20 g/L sucrose, pH 5.8. Solidified with 2 g/L Gelgro.
2. Bombardment of plasmid DNA and modified ssDNA-SCNAs into calli based on the method used by (Gordon-Kamm et. al., 1990).
3. Transfer calli to growth condition as described in example 2A, with final concentration of 2.5 mg/L Bialaphos in the medium (B0178 Gold Biotechnology, 1328 Ashby Rd., St. Louis, MO 63132 U.S.A.).
4. Calli are moved into new medium every 2 weeks.
5. Calli grown for 2 month on Bialaphos are resistant to the herbicide and can be subjected to PCR analysis or regeneration.
6. Regenerated plants are both resistant to Basta and have reduced levels of phytate.

### Detection and Analysis:

Calli bombarded with the modified ssDNA-SCNA, the plasmid encoding the programmable molecular complex protein moiety and the donor DNA containing bar resistance CDS expression cassette are grown on regeneration medium containing 2.5 mg/L Bialaphos. Only calli that include cells where the bar gene coding sequence are integrated into the IPK1 locus through HR are able to grow under these conditions, therefore, plant material still proliferating after 1 month on this medium is deemed genome-modified as desired.

By this design, while the bar resistance cassette integrates into the genome by HR to function properly, the Corynebacterium diphtheria toxin A (DT-A) cassette is an autonomous cassette that expresses the DT-A under heat shock (HS) conditions (42 degrees C.). Thus, for further analysis, calli is split into HS induced calli and uninduced calli. Only calli which contain a perfect HR event do not express the DT-A. Calli that contain randomly integrated plasmid, which contains both the donor DNA and the DT-A cassette express the DT-A and consequently die.

Further, calli are subjected to PCR analysis using the primers 1F and 1R shown in example 2A, followed by digestion of the product, as above, with BspHI.

### Donor Sequence for Experiment 2B:

The Donor plasmid contains both a bar resistance cassette, to be inserted into the IPK1 cleavage site, and a DT-A cassette which should not recombine into the IPK1 locus, as a non-specific integration event marker: The bar resistance cassette is flanked by sequences homologous to IPK1 (nts. 1-621 and 2338-2988 of SEQ ID NO:47) necessary for HR, while the DT-A cassette is located outside the homologous sequence flanked site. The bar cassette (nts. 622-2337 of SEQ ID NO: 47) contains a CaMv 35S constitutive promoter; the Streptomyces hygroscopicus bar gene CDS for phosphinothricin acetyl transferase conferring glufosinate ammonium resistance (nts. 1526-2078 of SEQ ID NO:47); and the NOS terminator—downstream from the bar CDS. The entire 2B Donor sequence is set forth in SEQ ID NO: 47.

On the same plasmid, a second cassette encoding diphtheria toxin A, DT-A, (from GenBank: AB535096.1) under the control of a Heat-shock inducible promoter (HS-Promotor of Arabidopsis HSP18.2 from GenBank: X17295.1) and terminated with a NOS terminator has the sequence as set forth in SEQ ID NO: 48.

### Example 3. Induction of Predetermined Chromosomal Double Strand Breaks (DSBs) in Living Cells of Arabidopsis

The enzyme Phytoene Desaturase (PDS) is involved in the conversion of phytoene to zeta-carotene in carotenoid biosynthesis. Disruption of Arabidopsis phytoene desaturase results in albino and dwarf phenotypes. This phenotype is explained by impaired chlorophyll, carotenoid, and gibberellin biosynthesis. Thus, a mutation in this gene is phenotypically detectable.

### Experiment 3A

In this example, a chromosomal double-strand break (DSB) in the endogenous PDS gene is specifically induced in order to create a point mutation through a frameshift, thus knocking out the function of the gene by utilizing the NHEJ endogenous pathway.

### Experiment 3B

In this example, a chromosomal double-strand break (DSB) specifically induced in the endogenous PDS gene in order to create an Insertion of a mCherry Donor sequence into an endogenous PDS sequence to knock out PDS by assisted homologous recombination using the programmable molecular complex.

For examples 3A-3B, an Arabidopsis protoplast based bioassay is used. In this bioassay the protoplasts are delivered with a plasmid expressing the protein moiety of the molecular complex in-vivo and co-delivered with a pair of ssDNA Specificity Conferring Nucleic Acids (SCNA) modified, in this example, with a terminal Fluorescein (6-carboxy-Fluorescein, 6-FAM), each SCNA having such a modification at either the 3′-terminus or the 5′-terminus (/36-FAM/ and /56-FAM/, respectfully). A second modification for exonuclease protection, such as phosphorothioate, is added at the opposite terminus, as may internal phosphorothioate bonds for endonuclease protection. In this example, the coding sequences for the Protein Moiety and the Donor DNA are concomitantly delivered on a single plasmid using a PEG transfection protocol (Wu et. al., 2009). Modified ssDNA SCNAs are synthetically produced and delivered together with the plasmid using PEG as above.

### Protein Sequence and Properties

In this example, the protein moiety, encoded on a plasmid, contains an amino-acid sequence adapted from a FokI nuclease domain as the Functional Domain; an amino-acid sequence adapted from anti-Fluorescein single-chain variable fragment (scFv) immunoglobin (Protein Data Bank accession codes 1X9Q, 1FLR H), as Linking Domain; an SV40NLS (PKKKRKV: SEQ ID NO: 3) as a nuclear localization domain and a ˜15 Å inter-domain connector (GGSGG: SEQ ID NO: 7).

Thus, the protein moiety of the molecular complex described in this example has the amino-acid sequence as set forth in SEQ ID NO: 49 and is encoded by the nucleotide sequence as set forth in SEQ ID NO:50.

The specificity-conferring nucleic acid (SCNA) of this example is modified by the addition of a Fluorescein-ScFv/6-FAM, 6-carboxyfluorescein—Fluorescein dT which includes a C6-linker to one end of each SCNA.

### SCNA Properties and Sequence

The design of the SCNA is essentially as described in Example 1. The length of the SCNA of the complementary, target-base-pairing oligonucleotide is preferentially at least 18 bases. The SCNA can also contain a small number (e.g. 1-6, in one example 6, in other example, 2) of non-target-base-pairing nucleotides (N's) of any sequence composition that serve as a spacer between the 6-FAM terminal-modifier and the target-complementary nucleotides.

### Target Sequence:

The target sequence is: GTCCTGCTAAGCCTTTGAAAG (SEQ ID NO: 51), Located on Exon 2 of the Arabidopsis PDS Sequence (GI:5280985, gene dl3145c, protein id=“CAB10200.1).

### SCNA Sequence Options:

SCNAs may be targeted to either strand, thus, for the shown target, 4 SCNA pairing options exist:

Sense (S) SCNAs:
PDS-SL1-846:
GCATCCTTCCGTAGTGCTCCTCNNNNNN/36-FAM/
(the nucleic acids only are designated herein as
SEQ ID NO: 52)
PDS-SR1-868:
/56-FAM/NNNNNNTTGTAATTGCTGGTGCTGGTAT
(the nucleic acids only are designated herein as
SEQ ID NO: 53)
Anti-sense (AS) SCNAs:
PDS-ASL1-846:
/56-FAM/NNNNNNGAGGAGCACTACGGAAGGATGC
(the nucleic acids only are designated herein as
SEQ ID NO: 54)
PDS-ASR1-868:
ATACCAGCACCAGCAATTACAANNNNNN/36-FAM/
(the nucleic acids only are designated herein as
SEQ ID NO: 217)
Mixed strand SCNAs:
PDS-SL1-846:
GCATCCTTCCGTAGTGCTCCTCNNNNNN/36-FAM/
(the nucleic acids only are designated herein as
SEQ ID NO: 52)
PDS-ASR1-868:
ATACCAGCACCAGCAATTACAANNNNNN/36-FAM/
(the nucleic acids only are designated herein as
SEQ ID NO: 217)
PDS-SR1-868:
/56-FAM/NNNNNNTTGTAATTGCTGGTGCTGGTAT
(the nucleic acids only are designated herein as
SEQ ID NO: 53)
PDS-ASL1-846:
/56-FAM/NNNNNNGAGGAGCACTACGGAAGGATGC
(the nucleic acids only are designated herein as
SEQ ID NO: 54)

A second set of paired “R” and “L” SCNAs combinations, employing a 24 bp target gap and a shorter SCNA linker according to the prediction results:

PDS-SL2-845:
TGCATCCTTCCGTAGTGCTCCTNN/36-FAM/
(the nucleic acids only are designated herein as
SEQ ID NO: 55)
PDS-SR2-870:
/56-FAM/NNGTAATTGCTGGTGCTGGTATGT
(the nucleic acids only are designated herein as
SEQ ID NO: 56)
PDS-ASL2-845:
/56-FAM/NNAGGAGCACTACGGAAGGATGCA
(the nucleic acids only are designated herein as
SEQ ID NO: 57)
PDS-ASR2-870:
ACATACCACCACCACCAATTACNN/36-FAM/
(the nucleic acids only are designated herein as
SEQ ID NO: 58)
PDS-SL2-845:
TGCATCCTTCCGTAGTGCTCCTNN/36-FAM/
(the nucleic acids only are designated herein as
SEQ ID NO: 55)
PDS-ASR2-870:
ACATACCAGCACCAGCAATTACNN/36-FAM/
(the nucleic acids only are designated herein as
SEQ ID NO: 58)
PDS-SR2-870:
/56-FAM/NNGTAATTGCTGGTGCTGGTATGT
(the nucleic acids only are designated herein as
SEQ ID NO: 56)
PDS-ASL2-845:
/56-FAM/NNAGGAGCACTACGGAAGGATGCA
(the nucleic acids only are designated herein as
SEQ ID NO: 57)
/56-FAM/ symbolizes a 5′-modification on the SCNA
ssDNA comprising of 6-FAM (6-carboxy-Fluorwscein).
/36-FAM/symbolizes a 3′-modification on the SCNA
ssDNA comprising of 6-FAM (3-carboxy-Fluoroscein).
N symbolizes any nucleotide

Donor sequence is DONOR PD-MCHERRY-S having the sequence as set forth in SEQ ID NO: 59 (mCherry encoding ORF is at nucleotides 662-1372 of SEQ ID NO:59).

### Delivery

Bioassay setup: Arabidopsis protoplast preparation is based on (Wu et. al., 2009) and is similar to that of example 1 with differences in the transfection step:

### Transfection:

1. Make fresh PEG sol for transfection in 2 ml tube
2. Pour off BSA from 6-well plates and dry
3. Mix ˜5×10\hat{ }4 protoplasts (2×10\hat{ }4-1×10\hat{ }5) in 0.2 ml MMg with a mixture of Donor plasmid DNA (where relevant), Protein Moiety expressing plasmid DNA and SCNAs ssDNA to a total of 30-40 microgram at RT in 15 ml round-bottom (snap-cap) tubes. Alternatively Donor DNA and Protein-moiety expressing DNA are constructed and delivered on a single plasmid.
4. Add equal volume (0.2 ml protoplasts+midiprep vol.) of fresh PEG sol
5. Incubate RT 5 min
6. Wash by slowly adding 3 ml W5, 1 ml at a time, and mixing
7. Centrifuge 100×g in swing-out 1 min
8. Repeat wash and pellet
9. Resuspend in 1 ml W5 solution
10. Pour into BSA-coated plates
11. Grow protoplasts under 16 hr day optimal light (150 microEinstein·m\hat{ }−2·s\hat{ }−1) at 22 degrees C., replacing media as needed.

### Analysis

In experiment 3A, DNA from pooled protoplasts is analyzed by PCR and restriction fragment analysis of the PCR product.

The PCR is conducted with the primers:

PCR Primer2F:

(SEQ ID NO: 60)

TGGTTGTGTTTGGGAATGTTTCT;
and
PCR Primer2R:

(SEQ ID NO: 61)

TATCCAAAAGATATCTTCCAGTAAAC

Abolishment of cleavage with the restriction enzyme DdeI in at least a portion of the amplified DNA indicates at least some successful gene targeting and directed mutation of the genomic template.

In experiment 3B a Donor DNA encoding mCherry is fused in frame to the endogenous PDS gene. mRNA produced from this gene encodes a disrupted PDS fused to a full mCherry immediately followed by a STOP codon (“PD-mCherry”). Protoplasts suspended in W5 solution are screened for mCherry activity 3 days after transfection using an automated flow-cytometer (FACS) machine. PDS-modified protoplasts are detected by FACS analysis, where an insertion of mCherry donor is detectable by mCherry fluorescence using a 561 nm excitation wavelength and detection of 590-630 nm emission. Threshold and compensation factors are set to exclude any false positives.

Further characterization in both experiments is achieved by regenerating protoplasts on suitable media and examining their subsequent phenotypic character, where bleached plants or calli indicate successful gene-targeting.

### Example 4. In-Vivo Genomic DNA Targeting and Gene-Replacement in the Dicotyledonous Plant Tobacco

Replacement of the ALS gene in tobacco and producing herbicide resistant plants: Acetolactate synthase (ALS) is an enzyme in the biosynthetic pathways of valine, leucine, and isoleucine in plants. Mutations in this gene result in resistance to several herbicides. For example, mutations in the SuRB gene in tobacco have been shown to provide the following herbicide resistances: S647T—imazaquin, P191A—chlorsulfuron, W568L—chlorsulfuron and imazaquin

In this example, the Tobacco ALS is targeted in order to replace the wild-type gene with a herbicide tolerant mutated version by assisted homologous-recombination mediated gene replacement.

Expression and assembly of the programmed molecular complex in tobacco plants is, performed here in two steps. Delivery of the protein moiety is achieved by infecting a Tobacco plant with a Tobacco Rattle Virus (TRV)-based viral protein expression vector such as a vector modified from pTRV2 (Vainstein et. al., 2011) for the delivery and expression of the programmable protein moiety in-planta.

Delivery of SCNA into plants expressing the protein moiety is achieved by infecting the plants with Agrobacterium carrying a T-DNA encoding both a pair of RNA-SCNAs and a Donor sequence.

The RNA-SCNAs in this example bind the Linking Domain of the Protein Moiety of the Molecular complex using the 20-mer boxB RNA hairpin binding sequence from bacteriophage Phi21 (SEQ ID NO: 62: 5′-UUCACCUCUAACCGGGUGAG-3′) as the “SCNA nucleotide motif” schematically exemplified in FIG. 1B.

The Linking Domain in this example is derived from the RNA-binding protein (RBP) bacteriophage Phi21 NProtein (SEQ ID NO: 63: N′-GTAKSRYKARRAELIAER-C′). In the example shown here the hairpin is not on the target but rather on the SCNA, and the action of the binding protein is thus not limited to a specific recognition site on the target RNA itself, but can be used to target any sequence, including DNA, depending exclusively on the variable SCNA target base-pairing sequence adjacent to the invariable RBP-binding hairpin.

The target nucleic acid (gene) in this example is SuRB (GenBank accession GI:19778) and the desired amino acid mutation is P191A—conferring chlorsulfuron resistance. Thus:

Unaltered original Sequence:

(SEQ ID NO: 64)

GGTCAAGTGCCACGTAGGATG
Induced Mutation:

(SEQ ID NO: 65)

GGTCAAGTGGCGCGCAGGATG

### The Sequence of the Components of the Protein Moiety:

### Components:

1. Bacteriophage Phi21 NProtein (SEQ ID NO: 63: GTAKSRYKARRAELIAER) at or near the N′ terminus as in the full-length N-protein the RNA-binding peptide is situated at the N-terminus.
2. FokI nuclease
3.

(SEQ ID NO: 66)
(VKSELEEKKSELRHKLKYVPHEYIELIEIARNSTQDRILEMKVMEFFM
KVYGYRGKHLGGSRKPDGAIYTVGSPIDYGVIVDTKAYSGGYNLPIGQA
DEMQRYVEENQTRNKHINPNEWWKVYPSSVTEFKFLFVSGHFKGNYKAQ
LTRLNHITNCNGAVLSVEELLIGGEMIKAGTLTLEEVRRKFNNGEINF)
SV40-NLS: (SEQ ID NO: 67: MPKKKRKV)

4. Interdomain connectors: various poly-amino-acid linkers are tested for optimal function of the programmed molecular complex.

Two options for protein assembly are tested in this example:
1. The first option, (as set forth in SEQ ID NO: 68), in which the Phi21 NProtein is assembled in the N′ terminus of the protein moiety of the programmable molecular construct and the nuclear localization signal, SV40NLS, is located at the C′ terminus and the interdomain linker is GGSGG (SEQ ID NO: 7). This protein assembly is encoded by the nucleic acid sequence as set forth in SEQ ID NO: 69.

Spatial measurements taken from computerized 3D models for the C′ Phi21 NP version in conjunction with the BoxB RNA hairpin system and the GGSGGESK (SEQ ID NO: 74) interdomain linker, as shown in this example, yielded that the expected optimal distance between SCNAs is, in the presence of a single “N” in the SCNA, about 26-30 nucleotides. Cleavage is predicted to occur about +2 nucleotides to left and to the right of the 13^{th}-17^{th} nucleotide, counting starts after the last nucleotide hybridizing with the SCNA on either side, taking into account the 4 base 5′ overhang created by dsDNA cleavage by the dimerized construct. This criterion suggests that if the targeted sequence is 28 nucleotide in this example:

AAAAAAAAAAYYYYYYYYYYXXXXXXXYYYYYYYYYYCCCCCCCCCC,

where Y+X represents the number of nucleotides between the SCNA base-pairing sites, then the designed SCNAs base-pair with areas A and C and the cleavage resulting in DSB is in or adjacent to the X area. The SCNAs can be complementary to either sense or antisense strands, but are chosen preferably to base-pair with the sense (untranscribed) sequence. Both SCNAs can base-pair with the same strand, as the protein moiety's position is situated at the “near end” of the SCNA as defined by the 5′ or 3′ modification of the primer being at the “near end”.

### SCNA Sequence Options:

SCNAs base-pair to sequences flanking the target site that is to be cleaved on either strand, thus, for the shown target, utilizing a 28 bp target gap: 4 SCNA pairing options exist:

Sense (S) SCNA pair:
SuRB_P191_SR1 586:
(SEQ ID NO: 70)
UUCACCUCUAACCGGGUGAGNGGUACUGAUGCUUUUCAGGAAA
SuRB_P191_SL1 557:
(SEQ ID NO: 71)
AUAGCGUCCCCAUUGUUGCUAUNUUCACCUCUAACCGGGUGAG
Antisense (AS) SCNA pair:
SuRB_P191_ASR1 586:
(SEQ ID NO: 72)
UUUCCUGAAAAGCAUCAGUACCNUUCACCUCUAACCGGGUGAG
SuRB_P191_ASL1 557:
(SEQ ID NO: 73)
UUCACCUCUAACCGGGUGAGNAUAGCAACAAUGGGGACGCUAU

And all combinations of sense and antisense pairs always choosing one Right (R) and one Left (L) SCNA:

The second option for protein assembly tested in this example, assembled with the Phi21 nProtein at the C′ of the protein and the SV40NLS at the N′ of the protein moiety. In this construct an interdomain connector of the sequence: GGSGGESK (SEQ ID NO: 74) is used:

The Assembled Phi21 NP-based programmable protein moiety of this example has the amino acid sequence as set forth in SEQ ID NO: 75 and is encoded by the nucleic acid sequence as set forth in SEQ ID NO: 76.

Results of spatial measurements taken from computerized 3D models for the C′ Phi21 NP version in conjunction with the BoxB RNA hairpin system and the GGSGGESK (SEQ ID NO: 74) interdomain linker, as used in this example, yielded that the expected optimal distance between SCNAs is, in the presence of 1 N in the SCNA, about 22-24 nucleotides. Cleavage is predicted to occur about +2 nucleotides to left and to the right of the 11^{th}, 12^{th} or 13^{th} nucleotide, counting from after the last nucleotide hybridizing with the SCNA on either side, taking into account the 4 base 5′ overhang created by dsDNA cleavage by the dimerized construct. This criterion suggests that if the targeted sequence is, for this 23 nucleotide example: AAAAAAAAAAYYYYYYYYXXXXXXXYYYYYYYYCCCCCCCCCC, where Y+X represents the number of nucleotides between the SCNA base-pairing sites, then the designed SCNAs base-pair with areas A and C and the cleavage resulting in DSB is in or adjacent to the X area. The SCNAs can be complementary to either sense or antisense strands, but are chosen preferably to base-pair with the sense (untranscribed) sequence. Both SCNAs can base-pair with the same strand, as the protein moiety's position is situated at the “near end” of the SCNA as defined by the 5′ or 3′ modification of the primer being at the “near end”.

### SCNA Sequence Options:

SCNAs base-pair to sequences flanking the target site to be cleaved on either strand, utilizing a 31 bp target gap, result in 4 SCNA pairing options:

Sense (S) SCNA pair:
SuRB_P191_SR1-588:
(SEQ ID NO: 77)
UUCACCUCUAACCGGGUGAGUACUGAUGCUUUUCAGGAAACU
SuRB_P191_SL1-556:
(SEQ ID NO: 78)
GAUAGCGUCCCCAUUGUUGCUAUUCACCUCUAACCGGGUGAG
Antisense (AS) SCNA pair:
SuRB_P191_ASR1-588:
(SEQ ID NO: 79)
AGUUUCCUGAAAAGCAUCAGUAUUCACCUCUAACCGGGUGAG
SuRB_P191_ASL1-556:
(SEQ ID NO: 80)
UUCACCUCUAACCGGGUGAGUAGCAACAAUGGGGACGCUAUC
Combinations of sense and antisense pairs:
SuRB_P191_SR1-588:
(SEQ ID NO: 77)
UUCACCUCUAACCGGGUGAGUACUGAUGCUUUUCAGGAAACU
SuRB_P191_ASL1-556:
(SEQ ID NO: 80)
UUCACCUCUAACCGGGUGAGUAGCAACAAUGGGGACGCUAUC
SuRB_P191_SL1-556:
(SEQ ID NO: 78)
GAUAGCGUCCCCAUUGUUGCUAUUCACCUCUAACCGGGUGAG
SuRB_P191_ASR1-588:
(SEQ ID NO: 79)
AGUUUCCUGAAAAGCAUCAGUAUUCACCUCUAACCGGGUGAG

A second set of paired “R” and “L” SCNAs combinations, employing a 23 bp target gap and a short (a single N) SCNA linker according to the prediction results:

Sense (S):
SURB_P191_SR2-584:
(SEQ ID NO: 81)
UUCACCUCUAACCGGGUGAGNUCGGUACUGAUGCUUUUCAGGA
SURB_P191_SL2-560:
(SEQ ID NO: 82)
GCGUCCCCAUUGUUGCUAUAACNUUCACCUCUAACCGGGUGAG
Antisense (AS): SuRB_P191_ASR2-584:
(SEQ ID NO: 83)
UCCUGAAAAGCAUCAGUACCGANUUCACCUCUAACCGGGUGAG
SuRB_P191_ASL2-560:
(SEQ ID NO: 84)
UUCACCUCUAACCGGGUGAGNGUUAUAGCAACAAUGGGGACGC

Or combinations of “R” and “L” SCNAs from the second set.
UUCACCUCUAACCGGGUGAG (SEQ ID NO:62) is the sequence of the 20-mer boxB RNA hairpin binding sequence from bacteriophage Phi21, and functions as the Linking-domain-binding segment of the SCNA (schematically marked as “SCNA nucleotide motif” in FIG. 1B).
Coding sequence of the ALS SURB CDS (unaltered) is set forth in SEQ ID NO: 85.
DONOR1 P191A: donor has altered nucleotide sequence to create a Proline to Alanine (P191A) mutation and to enable restriction enzyme analysis. The sequence of this donor is set forth as SEQ ID NO: 86. Altered sequence is as set forth in nucleotides 544-591 of SEQ ID NO: 86.

### Method:

In this example, natural host plants petunia, Nicotiana tabacum or N. benthamiana plants are first inoculated with a pTRV-based or pTRVdelta2b-based vector (Vainstein et. al., 2011), which is designed to express, in this example, the programmable molecular construct under the control of a viral subgenomic promoter. About 5-21 days after infection, plant leaves are collected and plant sap, used here as inoculum, is extracted by crushing the leaves in phosphate buffer (20 mM, pH 6.8), optionally supplemented with a nonionic wetting agent such as Silwet L-77 (about 0.015%). Clearing the solution by centrifugation and/or cheesecloth is optionally followed by 0.22 μm filtering. Filtering is necessary for injection into tissue-culture grown plants. Concomitantly, a portion of a leaf is analyzed for stability of the viral construct by extracting RNA, reverse transcribing the RNA using a primer 3′ from the foreign gene insertion site, amplifying the cDNA by PCR using primers spanning the foreign gene insertion site and electrophoresing side by side with a similarly PCR amplified pTRV plasmid originally used for inoculation. Target tobacco plants, about 1 month old, are then infected by lightly abrading their leaves with carborundum and rubbing the sap on the leaf surface. These plants may be grown in-vitro or otherwise. TRV-based self-replicating vector carrying the programmable molecular complex infects the plant and spreads systemically to leaves, meristems and non-inoculated tissues and organs. While still un-programmed the said complex is inactive as a nuclease.

Once the TRV-based self-replicating vector has spread systemically throughout the plant (about 5-7 days), hence, expressing the programmable protein moiety of the molecular complex, leaf-disks are excised under sterile conditions. Leaf disk transient expression is done in a manner similar to (Gallois & Marinho, 1995). Briefly, disks are vacuum infiltrated with a suitable strain of Agrobacterium (e.g. EHA105) pre-transformed with one binary plasmid (e.g. pRCS, pSOUP+pGreen, or other suitable binary vector) encoding between its RB and LB sequences one of the combinations of two SuRB_P191 SCNA transcripts shown above (see also FIG. 9 and FIG. 8A for schematic illustrations), under the control of two, identical or different, constitutive plant promoters such as CaMV 35S or NOS or OCS and carrying also the Donor1 P191A sequence. SCNAs are transcribed upon T-strand import into the cell, assemble with the programmable protein to form a programmed molecular complex which is then imported into the nucleus where it specifically cleaves a DSB in the SurB locus in the tobacco genomic DNA. Donor DNA from the T-DNA then recombines with the SurB gene near this DSB bringing about the desired mutation. Leaf disks are placed on selection medium containing 420 nM chlorsulfuron as described by Kochevenko (Kochevenko & Willmitzer, 2003) and in the detailed protocol below. Agrobacterium is killed with a suitable antibiotic (Carbenicillin 250 ug/ml+Vancomycin 250 ug/ml), and callus developing from leaf-disks is permitted to form shoots grown into herbicide resistant genome-modified plants. Regenerating plants are screened for chlorsulfuron resistance on Murashige and Skoog medium containing 420 nM chlorsulfuron as described by Kochevenko et. al. Only plants that grow on chlorsulfuron have an altered ALS gene, indicating that ALS was targeted by the programmed molecular complex and that the Donor was properly recombined into the correct location.

Analysis enabling resolving of successful gene-replacement events is achieved by conducting PCR on genomic DNA extracted from portions of Tobacco regenerants. On the altered sequence, the AgeI restriction enzyme site is abolished and BssHII and KpnI sites are added. Thus amplifying a PCR fragment encompassing the replacement site in the SuRB gene and digestion of the PCR fragment with AgeI, BssHII and KpnI provides a diagnostic pattern enabling recognition of successful gene replacement. These plants are further screened to eliminate those that have unwanted integrated T-DNA by DNA extraction and PCR amplification of a non-SuRB region of the SCNA-encoding T-DNA.

Detailed Agrobacterium transformation protocol:
1. Collect 2 ml overnight Agrobacterium culture (transformed with a binary plasmid encoding the SCNA transcripts and carrying the Donor DNA).
2. Resuspend in 4 ml Induction medium (1 L: 10.5 g K2HPO4, 4.5 g KH2PO4, 1 g (NH4)2SO4, 0.5 g NaCitrate, 1 g glucose, 4 g fructose, 4 g glycerol, 0.12 g MgSO4, 1.95 g MES pH5.6), add Acetosyringone to final concentration of 100 μM.
3. Grow at 30 degrees C. for 6 h.
4. Collect bacteria by centrifugation 3000 g 5 min.
5. Resuspend in infiltration medium (10 mM MgSO4, 10 mM MES pH5.6) containing 200 μM Acetosyringone to final OD600 0.4.
6. Take leaf discs of 4-12 mm diameter and incubate in the bacterial infiltration solution (step 5) for 30 min.
7. Place leaf discs on regeneration medium (1 L: 4.3 g MS, 30 g sucrose, 100 mg Myo-inositol, pH 5.6, 10 g Agar, add NAA and BA to final concentration of −100 microgram/L NAA and 3 mg/L BA). Incubate for 48 h at 20-25 degrees C.
8. Move leaf discs to new regeneration medium containing the antibiotic carbenicillin (0.3 mg) and the herbicide chlorsulfuron (420 nM). Move to new medium every 21 days.
9. Cut shoots above 10 mm and move to ½ MS medium for rooting (1 L: 2.15 g MS, 10 g Sucrose, 0.5 g MES pH=5.7 with KOH, 10 g Agar).

### Example 5. Targeted Chemical Modification of DNA Using a Programmed Molecular Complex

In this example, specific methylation of DNA in a predetermined location is tested.

DNA methylation is catalyzed by DNA methyltransferases, which transfer a methyl group (—CH3) from S-adenosyl-L-methionine to the C-5 position of cytosine residues. Three active DNA methyltransferases, DNMT1, DNMT3A, and DNMT3B, have been identified in humans and mice. Methylation in these examples is of DNA on the Cytosine of a CpG sequence. These enzymes belong to a class of S-adenosylmethionine-dependent methyltransferases (SAM or AdoMet-MTase), class I; AdoMet-MTases are enzymes that use S-adenosyl-L-methionine (SAM or AdoMet) as a substrate for methyltransfer, creating the product S-adenosyl-L-homocysteine (AdoHcy).

### DNMT3A

Both the DNMT1 and DNMT3 families of methyltransferases contain the highly conserved C-5 methyltransferase motifs at their C termini, but they show no sequence similarity in their N-terminal regions. DNMT3A also binds deacetylases and is recruited by a sequence-specific repressor to silence transcription. DNMT3A associates with the histone deacetylase HDAC1 using its ATRX homology domain. This domain of DNMT3A represents an independent transcriptional repressor domain whose silencing functions require HDAC activity. DNMT3A acts as a co-repressor protein carrying deacetylase activity and can be targeted to specific regulatory foci via its association with DNA-binding transcription. DNMT3A also cooperates with RP58 to repress transcription in a methylation-independent manner. In this example, methyltransferase activity is localized to a specific locus using SCNAs.

In this example a portion of the C′ of DNMT3A is used to construct a methyltransferase-based programmable molecular complex. The PWWP domains which target DNMT3A to pericentric heterochromatin, the Zinc-finger domains, the ADD domains, the ATRX region which causes its association the histone deacetylase HDAC1, and the whole regulatory N′-part of the protein are removed, keeping the region comprising the AdoMet_MTase region (www.uniprot.org Q9Y6K1). The C-terminus of DNMT3A and B contain the catalytic domain. In DNMT3A the active site is C710 (numbering is based on the translated GenBank accession AF067972.2).

DNMT3A forms a DNMT3L:DNMT3A:DNMT3A:DNMT3L heterotetramer complex. DNMT3L is inactive as a methylase, and DNMT3A can dimerize and is active without DNMT3L. DNMT3A is functional in the homodimeric form. The complex shows specific contacts at the DNMT3A homodimer interface (dimer interface) and dimerization brings two enzyme active sites separated by approximately one helical turn, in B-form DNA. Thus, a programmed molecular complex dimer localized to a specific locus by the SCNA, can bring about methylation of cytosines at CpG sites about 10-11 base pairs apart. To further restrict DNMT3A interactions with DNMTL, the mutation R729A in the C′ terminal AdoMet_MTase region is used in this example. The DNMT3A mutants that form dimers instead of tetramers on DNA are R771A, E733A, R729A, F732A, and Y735A.

In order to test the capability of the molecular complex of this example to perform directed specific methylation on a predetermined DNA sequence, a plasmid is used as the target nucleic acid. Directed methylation of different locations on the gene encoding the mCherry on both strands is tested on the pSAT6-mCherry plasmid by methylation sensitive restriction analysis.

Detection of transfected cells, is done by FACS analysis at wavelength 561 nm excitation and emission detected by 610/20 filter.

### Protein Moiety Construction:

In this example, the protein, encoded on the delivered plasmid, contains an amino-acid sequence adapted from the AdoMet_MTase region containing the catalytic site of a methyltransferase based on human DNA (cytosine-5)-methyltransferase 3A (DNMT3A PDB accession 2QRV is used to elucidate 3D structure). A mutation, R729 or R771 (based on the translated GenBank AF067972.2 numbering) is added to abolish tetramerization with the regulatory DNMTL without disrupting DNMT3A dimerization or reducing Kcat. The amino acid sequence (translated according to GenBank AF067972.2) of the methyltransferase region of this example is set forth in SEQ ID NO: 87 (DNMT3A AdoMet_MTase region R729A)

An amino-acid sequence adapted from anti-Fluorescein single-chain variable fragment (scFv) immunoglobin (Protein Data Bank accession codes 1X9Q, 1FLR H), is used in this example as Linking Domain; an SV40NLS (PKKKRKV: SEQ ID NO: 3) is used as a nuclear localization domain and inter-domain connectors such as a flexible inter-domain connector (SEQ ID NO. 14: GSLEGGSGG) are utilized in this example for their attachment. The protein moiety has the amino-acid sequence set forth in SEQ ID NO: 88, encoded by the nucleic acid sequence set forth as SEQ ID NO: 89:

The Target sequence for the methylation assay is based on a mCherry coding cassette cloned into the MCS site of pSAT6-MCS (AY818383.1 GI:56553596) and includes the nucleotide sequence as set forth in SEQ ID NO: 90. The mCherry coding sequence (cds) is as set forth in nucleotides 952-1671 of SEQ ID NO: 90. SCNA sequence used in this experiment:

SL898:
TCGAGCTCAAGCTTCGAATTCTNNNNNN/36-FAM/
(the nucleic acids only are designated herein as
SEQ ID NO: 91).
SR951:
/56-FAM/NNNNNNGATGGTGAGCAAGGGCGAGGAG
(the nucleic acids only are designated herein as
SEQ ID NO: 92).

3′- and 5′-6FAM (6 carboxy-Fluorescein) Linking-domain-binding-sites are labeled, by/36-FAM/and/56-FAM/respectively. Though one SCNA is sufficient for DNA methylation, it is possible to use more than one SCNA, spaced correctly to allow protein dimerization to enhance specificity.

### Experimental Procedure

A double transfection strategy is utilized to allow the expression of the protein moiety of the molecular complex before introduction of SCNAs and Target DNA.

Arabidopsis protoplast preparation is based on Wu (Wu et. al., 2009) and is similar to that of example 1 with differences in the transfection step:

### Transfection:

1. Make fresh PEG sol for transfection in 2 ml tube
2. Pour off BSA from 6-well plates and dry
3. Mix ˜5×10\hat{ }4 protoplasts (2×10\hat{ }4-1×10\hat{ }5) in 0.2 ml MMg with about 20 microgram Protein-Moiety-expressing plasmid DNA at RT in 15 ml round-bottom (snap-cap) tubes.
4. Add equal volume (0.2 ml protoplasts+midiprep vol.) of fresh PEG sol
5. Incubate RT° 5 min
6. Wash by slowly adding 3 ml W5, 1 ml at a time, and mixing
7. Centrifuge 100×g in swing-out 1 min
8. Repeat wash and pellet
9. Resuspend in 1 ml W5
10. Pour into BSA-coated plates
11. Grow protoplasts under 16 hr day optimal light (150 microEinstein·m\hat{ }−2·s\hat{ }−1) at 22 degrees C., replacing media as needed.
12. About 16 Hrs. later, retransfection of these cells is done, by repetition of steps 1-11 replacing the plasmid of step 3 with plasmid encoding the mCherry Target and with relevant SCNAs (total about 20 microgram).
13. mCherry expression and methylation status of extracted plasmids is analyzed 48 h later.

### Analysis

Analysis of CpG methylation status of target DNA are performed by two methods:
A) Digested DNA from pooled protoplasts is analyzed by PCR amplification. Digestion is performed using the methylation sensitive restriction enzymes SmaI (CCCGGG), SalI (GTCGAC) or SacII (CCGCGG). The SmaI, SalI, SacII cluster is used as a CpG site for the methylase. CpG dinucleotides underlined. Methylated DNA does not cleave with these enzymes. Thus, the MCS sequence spanning the cleavage sites of these enzymes is amplified and the product measured by Quantitative PCR returning a measure of the methylation efficiency versus samples lacking components of the molecular complex or deliberately containing non-specific SCNAs, scarcely amplified due to complete cleavage resulting from non-methylation.
B) DNA from pooled protoplasts is converted by bisulphite prior to PCR amplification, cloning and sequencing, to analyze the methylation status of a number of target and non-target control sequences. Bisulphite sequencing is done as described in the EZ DNA Methylation-Gold Kit (ZYMO, USA) suitable for methylated DNA detection and is used for further analysis.

### Example 6. Targeted Genome Modification in Humans: CCR5 Gene Deletion in Human Hematopoietic Stem Cells

C-C chemokine receptor type 5 (CCR5, GenBank Acc. Nr. NT_022517.18) is a chemokine receptor expressed and displayed on the surface of T cells, macrophages, dendritic cells and microglia. A mutation of this gene—CCR5-Δ32, which consists of a 32 base deletion, results in a frame-shift mutation which introduces 31 new amino-acids at the C′-terminus of the truncated protein, and confers resistance to smallpox and some types of Human Immunodeficiency Virus (HIV). This allele is found in about 10% of Europeans but is rare in other groups.

In the following example, CCR5 or portions of this gene are deleted from hematopoietic stem cells (HSC) extracted from HIV infected patients that do not have the Δ32 allele.

The protein moiety is composed of a nuclease-based Functional Domain (modified FokI nuclease domain, as above) and an RNA-motif-binding Linking Domain (derived from the BIV TAT protein minimal BIV TAT peptide SGPRPRGTRGKGRRIRR (SEQ ID NO: 93) domain, where the linking domain of the protein moiety binds the particular RNA sequence UUCAGCUCGUGUAGCUCAUUAGCUCCGAGCU (SEQ ID NO: 94) which is the BIV TAR loop 1. Delivery of the nucleic acid encoding for the protein moiety is performed concomitantly with the delivery of the specificity conferring nucleic acid (SCNA) by Adenoviral vector, for their transient expression. Adenoviruses do not integrate into the host genome.

Upon introduction and expression in the target cells (HSC), the molecular complexes self-assemble on the CCR5 target gene, allowing the protein moieties to dimerize and cleave the CCR5 sequence, to cause a deletion of portions of this gene, as intended. Following this genetic modification, the thus created gene-modified HSCs, or their descendants are autologously retransplanted to the patient. Cells which have been modified are enriched by selection by removing CCR5 displaying cells prior to grafting. CCR5 mutated T-cells and macrophages develop from these HSCs becoming resistant to HIV infection. Most of the Adenovirus and the molecular complex components clear from the HSCs before grafting, having completed their function.

Functionally preventing the display of CCR5 can be achieved through this system in several different ways, using different SCNA types and locations, as detailed below:

In the Δ32 allele, 32 nucleotides of the 3′ of the CCR5 CDS are missing, resulting in a frame-shift deletion. The deleted sequence is: TTCCATACAGTCAGTATCAATTCTGGAAGAA (SEQ ID NO: 95). To delete this sequence from CCR5 expressing cells, SCNAs derived from the following sequences (shown without Linking-domain-binding modification) are used:

(SEQ ID NO: 96)
ATCAATTCTGGAAGAATTTCCA;
(SEQ ID NO: 97)
TCATTACACCTGCAGCTCTCAT.

In this example, where the Linking Domain-binding modification on a transcribed SCNA utilizes the BIV TAR, the complete sequences of the SCNA sequences are:

SCNA distance option 1, Utilizing a 16b a and no SCNA internal “N” linker:

CCR5_D32_SR_3321:
(SEQ ID NO: 98)
UUCAGCUCGUGUAGCUCAUUAGCUCCGAGCUAUCAAUUCUGGAAGAAUUU
CCA
CCR5_D32_SL_3304:
(SEQ ID NO: 99)
UCAUUACACCUGCAGCUCUCAUUUCAGCUCGUGUAGCUCAUUAGCUCCGA
GCU

SCNA distance option 2, employing a 27 bp target gap and 2 “N” linker nucleotides:CCR5_D32_SR_3319:

CCR5_D32_SR_3319:
(SEQ ID NO: 100)
UUCAGCUCGUGUAGCUCAUUAGCUCCGAGCUNNGUAUCAAUUCUGGAAGA
AUUUC
CCR5_D32_SL_3291:
(SEQ ID NO: 101)
CAAAAAGAAGGUCUUCAUUACACNNUUCAGCUCGUGUAGCUCAUUAGCUC
CGAGCU

These SCNAs are designed to allow modification/cleavage in the TTTCCATACAGTCAGTATCAATTCTGGAAGAA target sequence (SEQ ID NO: 102). Cleavage and DSB formation mediated by these pairs alone can, in some cases, through endogenous mechanisms, cause a mutation that can lead to a frame shift. In order to make wider deletions in the CCR5 gene pairs of SCNAs targeting at least two targets on CCR5 are used:

Deletion of substantially all of the CCR5 coding sequence are induced by using CCR5-ATG region binding SCNAs and CCR5-STOP codon region binding SCNAs, concomitantly.

ATG SCNAs:
Targeted area between SCNAs (ATG underlined):
(SEQ ID NO: 103)
CAGGGTGGAACAAGATGGATTATCAAGTGTC.
SCNA distance option 1 utilizing a 31 bp target gap
and no SCNA internal ″N″ linker:
CCR5_SR_2779:
(SEQ ID NO: 104)
UUCAGCUCGUGUAGCUCAUUAGCUCCGAGCUAAGTCCAATCTATGACATC
AAT;
CCR5_SL_2747:
(SEQ ID NO: 105)
AAGATCACTTTTTATTTATGCAUUCAGCUCGUGUAGCUCAUUAGCUCCGA
GCU.
SCNA distance option 2, based on the computational
results, employing a 27 bp target gap and 2 ″N″
linker nucleotides:
CCR5_SR_2777:
(SEQ ID NO: 106)
UUCAGCUCGUGUAGCUCAUUAGCUCCGAGCUNNUCAAGUCCAAUCUAUGA
CAUCA
CCR5_SL_2749:
(SEQ ID NO: 107)
GAUCACUUUUUAUUUAUGCACANNUUCAGCUCGUGUAGCUCAUUAGCUCC
GAGCU
STOP SCNAs:
Targeted area between SCNAs (STOP codon
underlined):
(SEQ ID NO: 108)
ATATCTGTGGGCTTGTGACACGGACTCAAGT
SCNA distance option 1 Utilizing a 31 bp target gap
and no SCNA internal ″N″ linker:
CCR5_SR_3884:
(SEQ ID NO: 109)
UUCAGCUCGUGUAGCUCAUUAGCUCCGAGCUGGGCTGGTGACCCAGTCAG
AGT;
CCR5_SL_3802:
(SEQ ID NO: 110)
CCGATCCACTGGGGAGCAGGAAUUCAGCUCGUGUAGCUCAUUAGCUCCGA
GCU
SCNA distance option 2, based on computational
results, employing a 27 bp target gap and 2 ″N″
linker nucleotides:
CCR5_SR_3833:
(SEQ ID NO: 111)
UUCAGCUCGUGUAGCUCAUUAGCUCCGAGCUNNUGGGCUGGUGACCCAGU
CAGAG
CCR5_SL_3805:
(SEQ ID NO: 112)
AUCCACUGGGGAGCAGGAAAUANNUUCAGCUCGUGUAGCUCAUUAGCUCC
GAGCU

The protein moiety of the molecular complex is expressed via a nucleotide sequence carried in an Adenovirus-based expression system, such as, Adeno-X™ Adenoviral System 3 (Clontech Laboratories (CA, USA)) and used according to manufacturer instructions. Alternatively, the protein moiety is delivered by naked RNA transfection.

The Protein Moiety Amino-Acid Sequence for this Example:

Functional Domain: derived from the FokI nuclease subunit (as above).

Linking Domain: minimal BIV TAT peptide SGPRPRGTRGKGRRIRR (SEQ ID NO: 93) domain.

Cellular Localization Domain: Nuclear localization signal (NLS) domain of SV40 (SV40NLS).

FokI nuclease subunit:
(SEQ ID NO: 66)
VKSELEEKKSELRHKLKYVPHEYIELIEIARNSTQDRILEMKVMEFFMKV
YGYRGKHLGGSRKPDGAIYTVGSPIDYGVIVDTKAYSGGYNLPIGQADEM
QRYVEENQTRNKHINPNEWWKVYPSSVTEFKFLFVSGHFKGNYKAQLTRL
NHITNCNGAVLSVEELLIGGEMIKAGTLTLEEVRRKFNNGEINF;
SV40NLS:
(SEQ ID NO: 67)
MPKKKRKV;
BIV TAT peptide:
(SEQ ID NO: 93)
SGPRPRGTRGKGRRIRR.
Interdomain connector:
(SEQ ID NO: 113)
GSGGSGP

The Assembled BIV TAT—based programmable protein moiety of this example has the amino acid sequence as set forth in SEQ ID NO: 114, which is encoded by the nucleic acid sequence as set forth in SEQ ID NO: 115.

Spatial measurements taken from computerized 3D models for the BIV-TAT-TAR system with the GGSGGGP (SEQ ID NO: 116) interdomain linker, as used in this example, yielded that the expected optimal distance between SCNAs is, in the presence of 2 N's in the SCNA, is about 26-28 nucleotides. Cleavage is predicted to occur about +2 nucleotides to left and to the right of the 12^{th}, 13^{th} or 14^{th} nucleotide, counting starts after the last nucleotide hybridizing with the SCNA on either side, taking into account the 4 base 5′ overhang created by dsDNA cleavage by the dimerized construct. This criterion suggests that if, as in this example, the targeted sequence is, 27 nucleotides: AAAAAAAAAAYYYYYYYYYYXXXXXXXYYYYYYYYYYCCCCCCCCCC, where Y+X represents the number of nucleotides between the SCNA base-pairing sites, then the designed SCNAs base-pair with areas A and C and the cleavage resulting in DSB is in or adjacent to the X area (target site). The SCNAs can be complementary to either sense or antisense strands, but are preferably chosen to base-pair with the sense (untranscribed) sequence. Both SCNAs can base-pair with the same strand, as the protein moiety's position is situated at the “near end” of the SCNA as defined by the 5′ or 3′ modification of the primer being at the “near end”.

Detection and selection of CCR5 non-expressing/presenting cells vs. wild type CCR5 expressing cells is performed by FACS analysis, using a monoclonal mouse anti-Human CCR5 antibody (R&D systems Catalog nr. FABSP1).

### Example 7. Programmable Nucleic-Acid Base-Pairing Targeted Transcriptional Activator

In this example, a protoplast system in the monocot maize (Marrs & Urioste, 1995; Rhodes et. al., 1988) is used as a bioassay. In this system maize protoplasts are electroporated to introduce a plasmid for transient expression. These protoplasts may then be regenerated if so desired.

In this example, a protein moiety composed of the Gal4 transcriptional activator domain, excluding the UAS binding domain, and a linking domain composed of the anti-Fluorescein ScFV, together with a Fluorescein-modified SCNA, is used to activate the expression of a reporter gene. In this example, used here, the DNA binding domain of the Gal4 is removed and replaced with a Linking Domain of the protein moiety.

In the first example, two reporter plasmids are used, which can express GFP (option 1) or β-glucoronidase (GUS, option 2) only if a transcriptional activator is bound to a sequence upstream from a TATA box. In this example, this sequence is a 6×-UAS, known to be activated by Gal4 protein.

In the second example, the UAS sequences are removed from the target nucleic acid and the SCNA binds at minus 62 (6 2nt downstream from the TATA box), thus essentially attaining the same result but without any natural promoter. In the maize protoplast bioassay system the protein moiety shown below and the SCNA can be co-transfected using electroporation.

Protein moiety amino acid sequence: comprising an N′ nuclear-targeted Gal4 activation domain fused via an interdomain connector to an anti-Fluorescein ScFv is designated herein as SEQ ID NO: 132 and is encoded by the nucleotide sequence as set for the in SEQ ID NO: 157.

The first example utilizes a target plasmid with 6 UAS repeats:

The target plasmid contains, in the following order (5′->3′), a 6UAS promoter region followed by a TATA box and is designated herein in SEQ ID NO: 180:

GGACTGTAGAGGTTCCGGGTGACAGCCCTCCGACGGGTGACAGCCCTCCG
ACGGGTGACAGCCCTCCGAATTCTAGAGGATCCGGGTGACAGCCCTCCGA
CGGGTGACAGCCCTCCGACGGGTGACAGCCCTCCGAATTCGAGCTCGGTA
CCCGGGGATCTGTCGACCTCGATCGAGATCTTCGCAAGACCCTTCCTCTA
TATA;
A spacer having the sequence:
(SEQ ID NO: 192)
AGGAAGTTCATTTCATTTGGRGAGGACACGCTGAACC;
Option 1: A GFP coding sequence set forth in SEQ
ID NO: 193.
Option 2: A β-glucoronidase (GUS) coding sequence,
set forth in SEQ ID NO: 194.
a 35S-Terminator sequence:
(SEQ ID NO: 195)
GTCCGCAAAAATCACCAGTCTCTCTCTACAAATCTATCTCTCTCTATTTT
TCTCCAGAATAATGTGTGAGTAGTTCCCAGATAAGGGAATTAGGGTTCTT
ATAGGGTTTCGCTCATGTGTTGAGCATATAAGAAACCCTTAGTATGTATT
TGTATTTGTAAAATACTTCTATCAATAAAATTTCTAATTCCTAAAACCAA
AATCCAGTGAC

Two different orientations of SCNA are supplied in separate experiments to choose the more effective of the two: UAS-sequence binding SCNA

Sense:
CGGGTGACAGCCCTCCGANNNNNN/36-FAM/
(the nucleic acids only are set forth herein in
SEQ ID NO: 196)
Anti-sense:
/5-6FAM/NNNNNNTCGGAGGGCTGTCACCCG
(the nucleic acids only are set forth herein in
SEQ ID NO: 197)

The end modification of the SCNAs is 6-carboxy fluorescein (6FAM). 5′ or 3′ modification shown as /5-6FAM/ or /3-6FAM/ respectively. N represents any nucleotide.

The second example utilizes a target plasmid lacking a promoter for controlling expression of the reported gene:

The target plasmid contains, in the following order, a plasmid backbone sequence followed by a TATA box:

(SEQ ID NO: 198)
TCTTCGCTATTACGCCAGCTGGCGAAAGGGGGATGTGCTGCAAGGCGATT
AAGTTGGGTAACGCCAGGGTTTTCCCAGTCACGACGTTGTAAAACGACGG
CCAGTGCCACCCATAATACCCATAATAGCTGTTTGCCAACCGGTTCTATA
TA;
A spacer sequence (SEQ ID NO: 199):
AGGAAGTTCATTTCATTTGGRGAGGACACGCTGAACC;

Option 1: the GFP ORF, as set forth in SEQ ID NO: 200.
Option 2: β-glucoronidase (GUS) coding sequence, as set forth in SEQ ID NO: 201.

### a 35S Terminator Sequence (SEQ ID NO: 202):

Two different orientations of SCNA are used:

SCNA: options (minus 62):
GCCAGGGTTTTCCCAGTCACGANNNNNN/36-FAM/
(the nucleic acids only are set forth herein in
SEQ ID NO: 203)
/5-6FAM/NNNNNNTCGTGACTGGGAAAACCCTGGC
(the nucleic acids only are set forth herein in
SEQ ID NO: 204)

Maize protoplasts are tested for GFP expression (option 1) using microscopic or flow-cytometric methods. GFP positive cells indicate the functioning of the programmed complex. The percentage of GFP positive cells allows the calculation of relative efficiencies between experiments conducted to improve different parameters of the system. Absence of GFP in cells missing the proper components of the complex (e.g. by using control non-specific SCNAs) allows to measure the limits of specificity.

Maize protoplasts are tested for GUS expression (option 2) by staining the cells with X-Gluc in 0.45M mannitol and incubating overnight at 37° C., and detected using a microscope. GUS positive cells (stained blue) indicate the functioning of the programmed complex. The percentage of GUS positive cells allows us to calculate relative efficiencies between experiments conducted to improve different parameters of the system. Absence of GUS in cells missing the proper components of the complex (e.g. by using control non-specific SCNAs) allows us to measure the limits of specificity.

### Example 8: Gene-Targeting in Organellar DNA

In Eukaryotes, organelles such as mitochondria and plastids contain their own genomes. Furthermore, in plants, they may also contain sub-genomic circular DNAs. Modifying mitochondrial DNA can have implications for treatment of human disease and for agricultural uses, among others. Challenges for these modifications include, among other technical hurdles, the delivery and activation of a reasonably efficient, sequence-specific system necessary for gene-editing into the organelle.

### PCF in Petunia

Cytoplasmic male sterility (CMS) is a valuable plant trait used extensively by commercial seed companies as a method of protecting their seed lines. Thus it is advantageous to either repair CMS in existing lines or create CMS in new lines. Cytoplasmic male sterility can be due to the failure of plants to produce functional anthers, pollen, or male gametes as the result of specific nuclear and mitochondrial interactions. In the examples shown here a characterized cytoplasmic male sterility trait in petunia which is caused by a combined deletion and insertion into the atp9 gene in mitochondrial DNA which encodes the subunit 9 of an ATPase, is used. This results in disruption of the proton-translocating function of the mitochondrial ATPase multiprotein complex leading to male sterility.

The protein moiety of the programmable molecular complex of this example is designed to harbor a mitochondrial localization signal to ensure the localization of the programmed molecular complex inside the mitochondria. Other methods to transfer nucleic acids into mitochondria include the use of liposomes or electroporation. Plant mitochondria, and specifically in a plant from the solanaceae which includes Petunia, actively import DNA via the permeability transition pore complex. The process is restricted to double-strand DNA, but has no obvious sequence specificity. Donor sequences can be delivered, for example, either as linear purified PCR fragments, linearized plasmids, or as circular plasmids, depending on the method of delivery. Expression from plasmids, electroporated into isolated wheat mitochondria, for example, is very efficient when using a mitochondria-compatible promoter such as the 882 bp of T. timopheevi cox II mitochondrial promoter containing the initiation region described by (Hanic-Joyce and Gray, 1991).

Selection of cells containing a replacement or insertion event can be achieved by a Chloramphenicol resistance operon encoded in the Donor DNA.

In the following examples (8A-8C) the protein moiety comprises:

A Linking Domain derived from BIV TAT peptide comprising the amino-acid sequence

(SEQ ID NO: 93)
SGPRPRGTRGKGRRIRR;

A Functional Domain derived from FokI nuclease comprising the amino-acid sequence

(SEQ ID NO: 66)
VKSELEEKKSELRHKLKYVPHEYIELIEIARNSTQDRILEMKVMEFFMKV
YGYRGKHLGGSRKPDGAIYTVGSPIDYGVIVDTKAYSGGYNLPIGQADEM
QRYVEENQTRNKHINPNEWWKVYPSSVTEFKFLFVSGHFKGNYKAQLTRL
NHITNCNGAVLSVEELLIGGEMIKAGTLTLEEVRRKFNNGEINF;

a Cellular Localization Domain derived from the Arabidopsis Lipoic acid synthase and comprising the amino-acid sequence MHSRSALLYRFLRPASRCFSSSS (SEQ ID NO:6) which is a mitochondrial localization signal (MLS).

Interdomain connector:
(SEQ ID NO: 113)
GSGGSGP

The Assembled BIV TAT—based programmable protein moiety of this example has the amino acid sequence set forth in SEQ ID NO: 205, which is encoded by the nucleotide sequence set forth in SEQ ID NO:206.

The results of spatial measurements taken from computerized 3D models for the BIV-TAT-TAR system with the GGSGGGP (SEQ ID NO: 116) interdomain linker, of this example, show that the expected optimal distance between SCNAs, in the presence of 2 N's in the SCNA, is about 26-28 nucleotides. Cleavage is predicted to occur about +2 nucleotides to left and to the right of the 12^{th}, 13^{th} or 14^{th} nucleotide, counting starts after the last nucleotide hybridizing with the SCNA on either side, taking into account the 4 base 5′ overhang created by dsDNA cleavage by the dimerized construct. This criterion suggests that if the targeted sequence is, for example, the following 27 nucleotides: AAAAAAAAAAYYYYYYYYYYXXXXXXXYYYYYYYYYYCCCCCCCCCC, where Y+X represents the number of nucleotides between the SCNA base-pairing sites, then the designed SCNAs base-pair with areas A and C and the cleavage resulting in DSB is in or adjacent to the X area. The SCNAs can be complementary to either sense or antisense strands, but are chosen preferably to base-pair with the sense (untranscribed) sequence. Both SCNAs can base-pair with the same strand, as the protein moiety's position is situated at the “near end” of the SCNA as defined by the 5′ or 3′ modification of the primer being at the “near end”.

The SCNA Linking-Domain-binding RNA sequence used in this example is derived from the BIV TAR loop 1 comprising the nucleic acids sequence UUCAGCUCGUGUAGCUCAUUAGCUCCGAGCU (SEQ ID NO: 117). The SCNA may thus be either directly delivered to isolated mitochondria (by electroporation of mitochondria in the presence of a DNA encoding the SCNA under a bacterial promoter) or delivered to the cytoplasm (by Agrobacterium mediated transient transcription) and “pulled” into the mitochondria by the protein moiety bound to it and comprising an MLS.

After expression of the programmable molecular complex, mitochondria are isolated and a Donor DNA is transfected into the isolated mitochondria.

The following examples are performed, each having 2 options for SCNA distances:
1. Forming a CMS phenotype without a donor DNA (8A).
2. Targeting atp9 to form a pcf-like mutant using a Donor DNA with Chloramphenicol resistance (8B).
3. Repairing a pcf (CMS) phenotype, reforming ATP9 and restoring fertility and concomitantly using a Donor DNA with Chloramphenicol resistance (8C).
   Target nucleic acid sequences for these examples include:
   “ATP9”: Petunia x hybrida X Petunia axillaris subsp. parodii mitochondrial ATP synthase subunit 9, GenBank acc. Nr. Y00609.1 GI:297475.
   “pcf”: Cytoplasmic male sterility (CMS) in Petunia axillaris subsp. parodii, CMS-associated fusion protein (CMS-afp), NADH dehydrogenase subunit 3 (nad3), and ribosomal protein S12 (rps12) genes, complete cds; mitochondrial, GenBank acc. Nr. M16770.1 GI:1256946.

### Example 8A. Directed DNA-Mutation in Organellar DNA, without Organelle Isolation. Targeting ATP9 to Form a Mutation which Causes CMS by Creating a Non-Functional Protein ATP9 Protein

The SCNAs are designed to form a single DSB in the target site, that is repaired by the endogenous NHEJ repair pathway, creating frameshifts in part of the coding sequence.

ATP9 Target site:

(SEQ ID NO: 118)

GCAAAACAATTATTTGGTTATGCCATTTTGG.

SCNA distance option 1, 31 bp target gap:
ATP9 target site flanking SCNAs:

ATP9_ASL_705:
(SEQ ID NO: 119)
UUCAGCUCGUGUAGCUCAUUAGCUCCGAGCUCAAUGAUGGAUUUCGCGCC
ACG
ATP9_ASR_737:
(SEQ ID NO: 120)
UUAGCUUCGGUUAGAGCAAAGCUUCAGCUCGUGUAGCUCAUUAGCUCCGA
GCU

SCNA distance option 2, employing a 27 bp target gap:

ATP9_ASL_707:
(SEQ ID NO: 121)
UUCAGCUCGUGUAGCUCAUUAGCUCCGAGCUGCCAAUGAUGGAUUUCGCG
CCA
ATP9_ASR_735:
(SEQ ID NO: 122)
AGCUUCGGUUAGAGCAAAGCCCUUCAGCUCGUGUAGCUCAUUAGCUCCGA
GCU

Petunia leaves are inoculated using a standard leaf infiltration method as known in the art, with Agrobacterium harboring T-DNA derived from a binary vector plasmid encoding the protein moiety, and the RNA-SCNAs (as schematically shown in FIG. 8A). After transfection, the components of the Programmed molecular complex are expressed in the cytoplasm, self-assemble, and are then localize to the mitochondria by the mitochondrial import machinery, via the MLS exhibited on the surface of protein moiety. The programmed molecular complex (comprising the protein moiety and the targeting SCNA) then targets the ATP9 gene in mitochondrial DNA thus forming mutated mitochondria.

For analysis, 48 hours after the transfection, DNA is purified from the plants and the ATP9 sequence is amplified by PCR using primers:

ATP9atgF:

(SEQ ID NO: 123)

ATGTTAGAAGGTGCAAAATCAA
ATP9p2R:

(SEQ ID NO: 124)

CTAACGGACTTGGAATACGAAT

The PCR product is then subjected to CEL I Enzymatic Mutation Detection Assay (SURVEYOR Mutation Detection Kit (Transgenomics, USA)). This assay is used to evaluate the effectivity of mutation of mitochondrial DNA by gene targeting with a programmed molecular complex.

### Example 8B. Directed DNA-Insertion in Organellar DNA

In this example, ATP9 is targeted to form a pcf-like mutant by inserting a Donor DNA containing the selection marker chloramphenicol into the ATP9 locus.

Method: as in Example 8A, petunia leaves are inoculated using a standard leaf infiltration method with Agrobacterium harboring T-DNA derived from a binary vector plasmid encoding the protein moiety of the programmed molecular complex, and the SCNAs. After transfection, the components of the Programmed molecular complex are expressed in the cytoplasm, self-assemble, and are localize into the mitochondria by the mitochondrial import machinery via the MLS exhibited on the surface of protein moiety. After about 12-72 Hrs infiltrated leaves are used for mitochondrial preparation. A plasmid vector or a linear PCR product comprising the Donor DNA of this example, is delivered by electroporation into isolated mitochondria. The electroporated mitochondria are then transplanted into fresh Petunia protoplasts by microinjection. The injected protoplasts are regenerated on Chloramphenicol selection media allowing only the PCF like mitochondria to survive in the cells.

The 8B DONOR DNA (atp9 changed to pcf-like) is set forth in SEQ ID NO: 125:

### Results and Analysis:

The programmed molecular complex cleaves the atp9 gene in its coding sequence, downstream of the region homologous to pcf. This results in homologous recombination (HR) between the pcf-like Donor and the cleaved atp9 gene. A pcf male sterile genotype in the mitochondrial genome is thus recreated. Further, the donor contains a chloramphenicol resistance cassette allowing selection for mitochondria resistant to chloramphenicol. Injected protoplasts which are able to regenerate on selection media containing chloramphenicol contain the DNA-modified targeted mitochondria. Callus resulting from these protoplasts is capable of shoot differentiation, and ultimately whole plants are formed resulting in regenerated plants containing only the targeted mitochondria. Male sterile Petunia is thus achieved by regenerating plants from calli containing chloramphenicol resistant mitochondria.

### Example 8C. Directed DNA-Replacement in Organellar DNA

In this example, pcf mutant is targeted to form an active repaired ATP9 sequence using a Donor DNA containing a resistance to Chloramphenicol.

In this example, the Donor DNA is designed to be integrated by HR into the pcf locus, creating a STOP codon to recreate an intact ATP9 protein devoid of the superfluous amino-acid sequence causing the pcf disorder. A Chloramphenicol resistance cassette (AY230218.1 GI:30267504) in the Donor DNA is used for selection of repaired mitochondria. The CDS on the donor are in an operon based design. The chloramphenicol sequence is shown in underlined lowercase.

Method: A plasmid vector comprising the Donor DNA of this example, the SCNA shown in example 8C and the protein moiety of example 8A are delivered by electroporation into isolated mitochondria, in this example on a single plasmid similar in design to that schematically shown in FIG. 9.

Similarly to example 8B, the electroporated mitochondria are transplanted into Petunia protoplasts by microinjection. The protoplasts are shown on Chloramphenicol selection media. Callus resulting from these protoplasts is capable of shoot differentiation (Frearson et. al., 1973), and ultimately whole plants are formed resulting in regenerated plants containing only the targeted mitochondria. These petunia plants are screened for male-fertility.

### 8C Sequences:

Target site in pcf:

(SEQ ID NO: 126)

AGACTTACATCACGATGTCTTTTTCTTCGTT

SCNAs flanking target site:
SCNA distance option 1, 31 bp target gap:

CMS_ASL_704:
(SEQ ID NO: 127)
UUCAGCUCGUGUAGCUCAUUAGCUCCGAGCUGUUAUUUGUAUACCUAACA
CGG.
CMS_ASR_736:
(SEQ ID NO: 128)
AUACGAAAACCAAAAUCAGAAUUUCAGCUCGUGUAGCUCAUUAGCUCCGA
GCU.

SCNA distance option 2, based on computational results, employing a 27 bp target gap:

CMS_ASL_706
(SEQ ID NO: 129)
uucagcuCGUGUAGCUCAUUAGCUCCGagcuCUGUUAUUUGUAUACCUAA
CAC
CMS_ASR_734
(SEQ ID NO: 130)
ACGAAAACCAAAAUCAGAAUAAUUCAGCUCGUGUAGCUCAUUAGCUCCGA
GCU

The sequence of the 8C DONOR is as set forth in SEQ ID NO: 131.

### Example 9: Genomic Modification of Mammalian Cells: Preventing FAS Receptor Mediated Death

The FAS receptor (FasR) also known as apoptosis antigen 1 (APO-1, APT, TNFRSF6, CD95), is a protein that in humans is encoded by the TNFRSF6 gene located on chromosome 10 in humans (GenBank accession NC_000010 REGION: 90750288..90775542 GPC_000000034 VERSION NC_000010.10 GI:224589801). The Fas receptor is a death receptor displayed on the surface of cells that leads to programmed cell death (apoptosis) by forming the death-inducing signaling complex (DISC) upon ligand binding. Membrane-anchored Fas ligand trimer on the surface of an adjacent cell causes trimerization of Fas receptor. Fas ligand or FasL (CD95L) is a homotrimeric type II transmembrane protein. Soluble FasL is less active than its membrane-bound counterpart and does not induce receptor trimerization and DISC formation. Upon ensuing death domain (DD) aggregation, the receptor complex is internalized and initiates a cascade of events through caspases, eventually leading to DNA degradation, membrane blebbing, and other hallmarks of apoptosis. This event can also be mimicked by binding of an agonistic Fas antibody, used in the example here.

Eight splice variants of FasR are known, which are translated into seven isoforms of the protein. Apoptosis-inducing Fas receptor is dubbed isoform 1 and is a type 1 transmembrane protein. Fas protein has 319 amino acids, is divided into 3 domains: an extracellular domain, a transmembrane domain, and a cytoplasmic domain. The extracellular domain has 157 amino acids and is rich in cysteine residues. The transmembrane and cytoplasmic domains have 17 and 145 amino acids respectively. Exons 1 through 5 encode the extracelluar region which can interact with FasR trimer. Exon 6 encodes the transmembrane region. Exons 7-9 encode the intracellular region.

### Protein Sequence and Properties

The protein moiety is as described in Example 3.

Thus, the protein moiety of the molecular complex described in this example has the amino-acid sequence set forth in SEQ ID NO: 49.

The specificity-conferring nucleic acid (SCNA) of this example is modified by the addition of a Fluorescein-ScFv/6-FAM, 6-carboxyfluorescein—Fluorescein dT which includes a C6-linker to one end of each SCNA.

### SCNA Properties and Sequence

The length of the SCNA of the complementary, target-base-pairing oligonucleotide is preferentially at least 18 bases. The SCNA can also contain a small number (e.g. 0-6, in this example 6) of non-target-base-pairing nucleotides (N's) of any sequence composition that serve as a spacer between the 6-FAM terminal-modifier and the target-complementary nucleotides.

The results of spatial measurements taken from computerized 3D models for the anti-Fluorescein-ScFv-6-FAM system with the GGSGG (SEQ ID NO: 7) interdomain linker, as used in this example, yielded that the expected optimal distance between SCNAs is, in the presence of 2 N's in the SCNA, about 23-26 nucleotides. Cleavage is predicted to occur about +2 nucleotides to left and to the right of the 11^{th}, 12^{th} or 13^{th} nucleotide, counting from after the last nucleotide hybridizing with the SCNA on either side, taking into account the 4 base 5′ overhang created by dsDNA cleavage by the dimerized construct. This criterion suggests that if the targeted sequence is, for this 24 nucleotide example: AAAAAAAAAAYYYYYYYYYXXXXXXYYYYYYYYYcccccccccc, where Y+X represents the number of nucleotides between the SCNA base-pairing sites, then the designed SCNAs base-pair with areas A and C and the cleavage resulting in DSB is in or adjacent to the X area. The SCNAs can be complementary to either sense or antisense strands, but are chosen preferably to base-pair with the sense (untranscribed) sequence. Both SCNAs can base-pair with the same strand, as the protein moiety's position is situated at the “near end” of the SCNA as defined by the 5′ or 3′ modification of the primer being at the “near end”.

### Target Site Sequence:

The target sequences examples are:
A) Exon 1 starts at 347, target sequence is: GGGCATCTGGACCCTCCTACC (SEQ ID NO: 133)

### SCNAs:

SCNA distance option 1, 21 bp target gapSL351:

A*GGATTGCTCAACAACCATGCTNNNNNN/36-FAM/
(the nucleic acids only are set forth herein in
SEQ ID NO: 134)
SR373: /56-FAM/NNNNNNTCTGGTGAGCCCTCTCCTGCC*C
(the nucleic acids only are set forth herein in
SEQ ID NO: 135)

SCNA distance option 2, based on computational results, employing a 24 bp target gap and a shorter SCNA “N” linker:

SL349: G*GAGGATTGCTCAACAACCATGNN/36-FAM/
(the nucleic acids only are set forth herein in
SEQ ID NO: 136)
SR374: /56-FAM/NNCTGGTGAGCCCTCTCCTGCCC*G
(the nucleic acids only are set forth herein in
SEQ ID NO: 137)

Exon 2 starts at 12499, target sequence is: TACGTCTGTTGCTAGATTATC (SEQ ID NO: 138)
B)

### SCNAs:

SCNA distance option 1, 21 bp target gap:

SL12503: A*TGCTTTTATTTTACAGGTTCTNNNNNN/36-FAM/
(the nucleic acids only are set forth herein in
SEQ ID NO: 139)
SR12525: /56-FAM/NNNNNNGTCCAAAAGTGTTAATGCCCA*A
(the nucleic acids only are set forth herein in
SEQ ID NO: 140)

SCNA distance option 2, based on computational results, employing a 24 bp target gap and a shorter SCNA “N” linker:

SL12501: TCATGCTTTTATTTTACAGGTTNN/36-FAM/
(the nucleic acids only are set forth herein in
SEQ ID NO: 141)
SR12526: /56-FAM/NNTCCAAAAGTGTTAATGCCCAA*G
(the nucleic acids only are set forth herein in
SEQ ID NO: 142)

Exon 2 Target for restriction analysis: CAGTTGAGACTCAGAACTTGG (SEQ ID NO: 143)
C)

### SCNAS:

SCNA distance option 1, 21 bp target gap

SCNA distance option 1, 21bp target gap
SL12595: G*GAATTGAGGAAGACTGTTACTANNNNNN/36-FAM/
(the nucleic acids only are set forth herein in
SEQ ID NO: 144)
SR12617: /56-FAM/NNNNNNAAGGCCTGCATCATGATGGCCAATTC
T*C
(the nucleic acids only are set forth herein in
SEQ ID NO: 145)

SCNA distance option 2, based on computational results, employing a 24 bp target gap and a shorter SCNA “N” linker:

SL12594:
G*GAATTGAGGAAGACTGTTACTNN/36-FAM/
(the nucleic acids only are set forth herein in SEQ
ID NO: 146)
SR12619:
/56-FAM/NNGGCCTGCATCATGATGGCCAA*T
(the nucleic acids only are set forth herein in SEQ
ID NO: 147)

Primers for analysis of example C:

FAS_E2F:

(SEQ ID NO: 148)

CATGCTTTTATTTTACAG;
FAS_E2R:

(SEQ ID NO: 149)

CTGTGACTTTCACTGTAATC

PCR-amplification of the target with these primers forms (in unmodified DNA) a 227 bp PCR product digested with DdeI forming fragments of 127 bp and 100 bp. DdeI digestion is abolished by accurate targeting.

Exon 9 target:

(SEQ ID NO: 150)

CAATTGTGAATTCACATAGAA

D)

### SCNAs:

SCNA distance option 1, 21 bp target gap

SL24524:
G*GTGTCATATTATACAATATTTNNNNNN/36-FAM/
(the nucleic acids only are set forth herein in
SEQ ID NO: 151)
SR24546:
/56-FAM/NNNNNNAACATTAAATTATAATGTTTG*A
(the nucleic acids only are set forth herein in
SEQ ID NO: 152)

SCNA distance option 2, based on computational results, employing a 24 bp target gap and a shorter SCNA “N” linker:

SL24522:
T*TGGTGTCATATTATACAATATNN/36-FAM/
(the nucleic acids only are set forth herein in
SEQ ID NO: 153)
SR24547:
/56-FAM/NNACATTAAATTATAATGTTTGA*C
(the nucleic acids only are set forth herein in
SEQ ID NO: 154)

Primers for analysis of example D:

FAS_E9F
CTTTGTTTATAACTCTGAGAAG
(SEQ ID NO: 155)
FAS_E9R
TCAAAATGCTTTTGATGCCTGA
(the nucleic acids only are set forth herein in
SEQ ID NO: 156)

PCR-amplification of the target with these primers forms (in unmodified DNA) a 240 bp PCR product digested with EcoRI forming fragments of 134 bp and 106 bp. EcoRI digestion is abolished by accurate targeting.

/56-FAM/and/36-FAM/symbolize a 5′-modification or a 3′-modification respectively on the SCNA ssDNA comprising of 6-FAM (6-carboxy-Fluorescein). N symbolizes any nucleotide. Phosphorothioate-bonds are symbolized by an asterisk (*).

While each SCNA pair can cause a mutation that knocks out the FAS receptor, deletion of a whole stretch of DNA resulting from targeting more than one site in the gene can disable the activity of FASR outright. Thus, for example, using the SCNAs in examples A-C may result in mutations abolishing FasR activity, while using any of these SCNAs together with the SCNA of example D leads to a major genomic deletion that abolishes FasR activity.

### Assay:

A bioassay for detecting an induced specific mutation in Human genomic DNA is as follows: HeLa and Jurkat Cells are transfected with a plasmid encoding the protein moiety of the programmable molecular complex together with the relevant ssDNA SCNAs using the transfection agents (Mirus, USA) TransIT-HeLaMONSTER or TransIT-LT1 for formulating the plasmid DNA and TransIT-Oligo for formulating the SCNA ssDNA. Once incubated for the allotted time, both sets of formulated DNA-transfection-agent mixes are supplied simultaneously to the cells, to target chromosomal FasR. To determine the efficiency of gene-targeting cells are tested for their sensitivity to FasL in a protocol modified from (Kotlo et. al., 2003): Transfected cells are plated in duplicates 20-24 h prior to the treatment with a combination of 200 ng/ml anti-FasR agonistic antibody (Anti-Fas mAb, clone 2R2 Cat. No.: MC-121, Kamiya Biomedical Company, or monoclonal anti CD95 Clone 7C11, Cat. No.: PN IM2387 Beckman-Coulter) and optionally, a sensitizing agent such as Dicumarol 100 micromolar. Seventeen hours post-treatment, the number of viable, trypan blue excluding cells that remain attached to the plate following rinsing with PBS is determined or alternatively propidium iodide exclusion staining is done to evaluate intact living cells by Flow cytometry (FACS). Cells in which the FAS gene is targeted and disabled, do not go through a death-induction process, do not stain, but rather multiply. Thus, a comparison between induced, specifically targeted cells versus non-specifically targeted cells (e.g. no SCNAs or non-FAS SCNAs) evaluates gene targeting success in human cells. Surviving or FACS-sorted cell-lines are analysed by PCR amplification of genomic DNA in the targeted FasR regions followed by restriction fragment analysis and sequencing to identify induced mutations.

### Example 10: Editing Plasmid DNA Sequence In-Vivo. Antibiotic Resistance Modification

This example is for a bioassay suitable for testing and fine-tuning permutations in the basic design of the programmable molecular complex; for testing its application in different organisms or cells; for testing different delivery methods; and for testing the editing functions of mutation, replacement, deletion and insertion.

Bacterial selectable marker genes are used to determine the gene targeting efficiency when targeting plasmid DNA.

In these examples an Arabidopsis protoplast based bioassay is used. In this bioassay, protoplasts are delivered with the reporter system and the molecular complex on a plasmid, co-delivered with paired ssDNA SCNA modified with a terminal Digoxigenin (NHS Ester) (DIG), one SCNA having such a modification at the 3′-terminus and the other at the 5′-terminus. A second modification for exonuclease protection, such as phosphorothioate, may be added at the opposite terminus.

### Protein Sequence and Properties

The protein moiety is as described in Example 1.

In this example, the nucleic acid end-modification of the SCNA is an NHS-Ester linked Digoxigenin (DIG), attached to the 5′ or 3′ position of the oligonucleotide.

Amino-acid sequence (one letter code) of the protein moiety of the molecular complex (NLS-FokI-nuclease sequence With Digoxygenin ScFv is set forth in (SEQ ID NO: 12):

### SCNA Properties and Sequence

The length of the SCNA of the complementary, target-base-pairing oligonucleotide is preferentially at least 18 bases. The SCNA can also contain a small number (e.g. 1-6, in one example 6, in other example, 2) of non-target-base-pairing nucleotides (“N's”) of any sequence composition that serve as a spacer between the DIG-NHS terminal-modifier and the target-complementary nucleotides.

Results of spatial measurements taken from computerized 3D models for the anti-DIG-ScFv-NHS-Ester-DIG system with the GSLEGGSGG (SEQ ID NO: 14) interdomain linker, as shown in this example, yielded that the expected optimal distance between SCNAs is, in the presence of 2 N's in the SCNA, about 23-26 nucleotides. Cleavage is predicted to occur about +2 nucleotides to left and to the right of the 11^{th}, 12^{th} or 13^{th} nucleotide, counting from after the last nucleotide hybridizing with the SCNA on either side, taking into account the 4 base 5′ overhang created by dsDNA cleavage by the dimerized construct. This criterion suggests that if the targeted sequence is, for this 24 nucleotide example: AAAAAAAAAAYYYYYYYYYXXXXXXYYYYYYYYYCCCCCCCCCC, where Y+X represents the number of nucleotides between the SCNA base-pairing sites, then the designed SCNAs base-pair with areas A and C and the cleavage resulting in DSB is in or adjacent to the X area. The SCNAs can be complementary to either sense or antisense strands, but are chosen preferably to base-pair with the sense (untranscribed) sequence. Both SCNAs can base-pair with the same strand, as the protein moiety's position is situated at the “near end” of the SCNA as defined by the 5′ or 3′ modification of the primer being at the “near end”.

### Detection Assay:

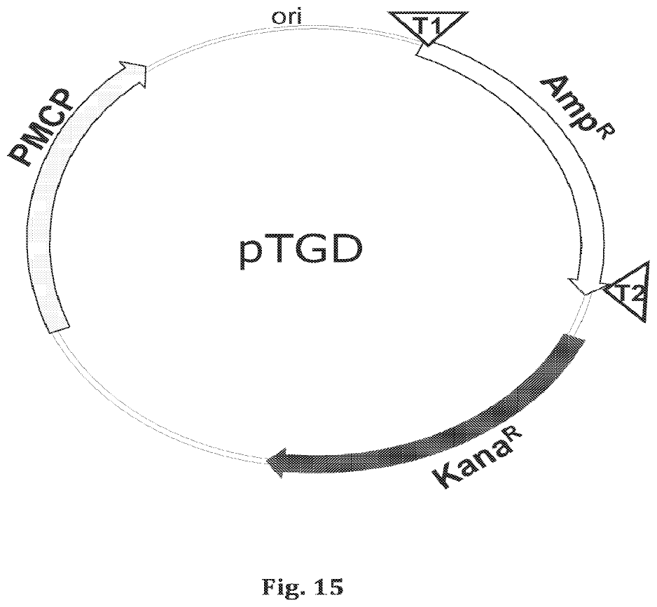

The target plasmid pTGD (schematically represented in FIG. 15) comprises 4 main sections:
1. The target ampicillin resistance cassette (AmpR).
2. Constitutive selection Kanamycin (Km) resistance cassette (KanR).
3. Origin of replication (ori).
4. The programmable molecular complex protein moiety encoding sequence cassette (PMCP) including a promoter suitable for the test organism, in this example, plants.
5. T1 and T2—target sequences 1 and 2.

This plasmid multiplies in bacterial cells such as E. coli cells. In this example the SCNAs, the target plasmid pTGD encoding the programmable molecular complex protein moiety and a donor DNA (in examples 10B, 1° C.) are delivered into Arabidopsis protoplasts. 48 hours after transfection, DNA is extracted from the transfected protoplasts (Kit A1120 Promega Corp.) and transformed into E. coli bacterial competent cells (Kit L3002 Promega Corp.). The transfected bacteria are spread on LB medium containing Kanamycin in a concentration of 100 microgram/ml. Colonies are grown for about 16 h at 37 degrees C. The colonies are then transferred in replica to Ampicillin (100 microgram/ml) or Tetracycline (100 microgram/ml) LB plates and grown for another 16 h at 37 degrees C.

### Analysis:

Colonies from each replica are counted. Number of Kanamycin resistant colonies suggests total plasmid number which also represents total target number. Colonies which are not resistant to Ampicillin are colonies that contain a plasmid successfully targeted validating the editing functions of “Mutation” or “Deletion”. Colonies resistant to Tetracycline but not to Ampicillin represent integration of the donor DNA into the target plasmid by NHEJ validating the editing function of “Replacement”. Colonies that are resistant to both Ampicillin and Tetracycline are colonies containing plasmids that were targeted, had the donor integrated into the Ampicillin target sequence, but did not replace it validating the editing function of “Insertion”.

Plasmids are then subjected to PCR and sequence analysis for verification of the results with the primers:

A961F:

(SEQ ID NO: 158)

TAGGGCGCTGGCAAGTGTAG
A2161R:

(SEQ ID NO: 159)

CATAACACCCCTTGTATTAC

### Experiments

### Example 10A—Targeted Mutation in the AMPR Cassette

The detection assay is performed essentially as described above (“detection assay”) with the following additional details: pTGD plasmid is transfected together with SCNAs flanking target sequence 1 (SEQ ID NO: 161) to Arabidopsis protoplasts. DNA is purified and transformed into E. coli competent cells which are spread on LB Kan medium. A replica is made on LB AMP plates. Colonies that lost resistance to AMP contain a targeted plasmid.

### Example 10B

The detection assay is performed essentially as described above (“detection assay”) with the following additional details: pTGD plasmid is transfected together with SCNAs flanking target sequence 1 and a linear dsDNA Tetracycline (Tet) donor, produced as a PCR product, into Arabidopsis protoplasts. DNA is purified and transformed into E. coli competent cells which are spread on LB Km medium. A replica is made on both LB AMP and on LB Tet plates. Colonies that lost resistance to AMP contain a targeted plasmid. Colonies resistant to Tet represent plasmids containing specifically integrated donor DNA.

### Example 10C

The detection assay is performed essentially as described above (“detection assay”) with the following additional details: pTGD plasmid is transfected together with SCNAs directed against target sequence 1 and SCNAs against target sequence 2 (SEQ ID NO: 170), together with the Tetracycline (Tet) donor DNA to Arabidopsis protoplasts. DNA is purified and transformed into E. coli competent cells which are spread on LB Km medium. A replica is made on LB AMP and on LB Tet plates. Colonies that lose resistance to AMP contain a targeted plasmid. Tet resistant colonies represent specifically integrated donor DNA. The AMP sensitive colonies are subjected to PCR analysis with primers A961F and A2161R.

Colonies that contain a plasmid incorporating the Tet donor (ca. 1.9 Kb) instead of the AMP (ca. 860 bp) target sequence demonstrate gene replacement events.

Colonies sensitive to both AMP and to Tet demonstrate gene deletion through NHEJ.

Colonies resistant to both Tet and AMP contain a plasmid incorporating the TetR donor without deletion of the Amp resistance cassette and demonstrate targeted donor integration or “insertion”.

### Delivery

Bioassay setup: Arabidopsis protoplast preparation is based on Wu et. al. (2009), and is similar to that of example 1 with differences in the transfection step:

### Transfection:

1. Make fresh PEG sol for transfection in 2 ml tube
2. Pour off BSA from 6-well plates and dry
3. Mix ˜5×10\hat{ }4 protoplasts (2×10\hat{ }4-1×10\hat{ }5) in 0.2 ml MMg with a mixture of plasmid comprising the Target plasmid DNA and the Protein Moiety expressing DNA, the ssDNA SCNAs and the linear dsDNA Donor to a total of 30-40 microgram at room temperature in 15 ml round-bottom (snap-cap) tubes.
4. Add equal volume (0.2 ml protoplasts+midiprep vol.) of fresh PEG sol
5. Incubate RT° 5 min
6. Wash by slowly adding 3 ml W5, 1 ml at a time, and mixing
7. Centrifuge 100×g in swing-out 1 min
8. Repeat wash and pellet
9. Resuspend in 1 ml W5 solution.
10. Pour into BSA-coated plates
11. Grow protoplasts under 16 hr day optimal light (150 microEinstein·m\hat{ }−2·s\hat{ }−1) at 22 degrees C., replacing media as needed.
   Protoplasts are then subjected to DNA extraction as described in the Detection Assay.
   Targeted AmpR Cassette is as set forth in SEQ ID NO: 160.
   SCNA pairs are chosen one left (L) and one right (R) irrespective of sense (S) or antisense (AS) strand: Choice of SCNA pair combination is a tested parameter in the experiment.
   Target sequence T1 on AMPR cassette: TATGAGTATTCAACATTTCCG (SEQ ID NO: 161) (ATG start codon is underlined)

### Set 1 of AMP Targeting SCNAs:

### Option 1—Utilizing a 21 bp Target Gap:

pTGD_130_SL:
A*ATAATATTGAAAAAGGAAGAGNNNNNN/3DIGN/
(the nucleic acids only are set forth herein in
SEQ ID NO: 162)
pTGD_152_SR:
/5DIGN/NNNNNNTGTCGCCCTTATTCCCTTTTT*T
(the nucleic acids only are set forth herein in
SEQ ID NO: 163)
pTGD_130_ASL:
/5DIGN/NNNNNNCTCTTCCTTTTTCAATATTAT*T
(the nucleic acids only are set forth herein in
SEQ ID NO: 164)
pTGD_152_ASR:
A*AAAAAGGGAATAAGGGCGACANNNNNN/3DIGN/
(the nucleic acids only are set forth herein in
SEQ ID NO: 165)

### Option 2—Paired Combinations, Employing a 24 bp Target Gap and a Shorter SCNA Linker According to the Prediction Results: AMP_129_SL:

C*AATAATATTGAAAAAGGAAGANN/3DIGN/
(the nucleic acids only are set forth herein in
SEQ ID NO: 166)
AMP_154_SR:
/5DIGN/NNTCGCCCTTATTCCCTTTTTTG*C
(the nucleic acids only are set forth herein in
SEQ ID NO: 167)
AMP_129_ASL:
/5DIGN/NNTCTTCCTTTTTCAATATTATT*G
(the nucleic acids only are set forth herein in
SEQ ID NO: 168)
AMP_154_ASR:
G*CAAAAAAGGGAATAAGGGCGANN/3DIGN/
(the nucleic acids only are set forth herein in
SEQ ID NO: 169)
Target sequence T2 on AMPR cassette:
AGCATTGGTAACTGTCAGACC
(SEQ ID NO: 170)

### Set 2 of AMP Targeting SCNAs:

### Option 1 Utilizing a 21 bp Target Gap:

pTGD_981_SL:
G*AGATAGGTGCCTCACTGATTANNNNNN/3DIGN/
(the nucleic acids only are set forth herein in
SEQ ID NO: 171)
pTGD_1003_SR:
/5DIGN/NNNNNNAAGTTTACTCATATATACTTT*A
(the nucleic acids only are set forth herein in
SEQ ID NO: 172)
pTGD_981_ASL:
/5DIGN/NNNNNNTAATCAGTGAGGCACCTATCT*C
(the nucleic acids only are set forth herein in
SEQ ID NO: 173)
pTGD_1003_ASR:
T*AAAGTATATATGAGTAAACTTNNNNNN/3DIGN/
(the nucleic acids only are set forth herein in
SEQ ID NO: 174)

### Option 2 Paired Combinations, Employing a 24 bp Target Gap and a Shorter SCNA Linker According to the Prediction Results:

AMP_980_SL:
T*GAGATAGGTGCCTCACTGATTNN/3DIGN/
(the nucleic acids only are set forth herein in
SEQ ID NO: 175)
AMP_1005_SR:
/5DIGN/NNGTTTACTCATATATACTTTAG*A
(the nucleic acids only are set forth herein in
SEQ ID NO: 176)
AMP_980_ASL:
/5DIGN/NNAATCAGTGAGGCACCTATCTC*A
(the nucleic acids only are set forth herein in
SEQ ID NO: 177)
AMP_1005_ASR:
T*CTAAAGTATATATGAGTAAACNN/3DIGN/
the nucleic acids only are set forth herein in
SEQ ID NO: 178)

### Donor:

Donor sequence encoding Tetracycline resistance from Cloning vector pSoup, EU048870.1 GI:155733614 is as set forth in SEQ ID NO: 179.

### Example 11: Construction of the Programmable Molecular Complex to Act with a Pair of Connected SCNA Sequences

In this example, the programmable molecular complex is designed to operate with a single nucleic acid molecule incorporating dual target sequence binding nucleic acid sequences, here designated as a connected pair of Specificity Conferring Nucleic Acid sequences (SCNA sequences) as schematically illustrated in FIGS. 4A and 4B.

In this example, a disrupted GFP target sequence is repaired by removal or mutation of a STOP codon. The resulting cleavage of the predetermined Target GFP leads to point mutation that may restore GFP activity.

In these examples, an Arabidopsis protoplast based bioassay, in which the protoplasts are delivered with the reporter system (target plasmid), protein moiety expressing plasmid, co-delivered with either: For example 12A (schematically illustrated in FIG. 4A)—A nucleic acid encoding an RNA, RNA composed of two SCNA sequences modified, in this example, by the 20-mer boxB RNA hairpin binding sequence from bacteriophage Phi21 (SEQ ID NO: 62: 5′-UUCACCUCUAACCGGGUGAG-3′) and an “SCNA Connector”, a non-target hybridizing stretch of nucleotides of undefined sequence or length. One SCNA having such a modification at the 3′-terminus and the other at the 5′-terminus of the RNA molecule. The RNA-SCNAs in this example bind the Linking Domain of the Protein Moiety of the two Molecular complexes using the 20-mer boxB RNA hairpin binding sequence from bacteriophage Phi21 (5′-UUCACCUCUAACCGGGUGAG-3′(SEQ ID NO: 62), or: In example 11B schematically illustrated in FIG. 4B) a modified ssDNA SCNA containing sequence, in this example, modified on both the 5′ and the 3′ termini by addition of terminal Digoxigenin (NHS Ester) (DIG) molecules and an “SCNA Connector”, a non-target hybridizing stretch of nucleotides of undefined sequence or length.

### Protein Sequence and Properties

The protein moiety in example 11A, contains an amino-acid sequence derived from a FokI nuclease domain as the Functional Domain, Linking Domain in derived from the RNA-binding protein (RBP) bacteriophage Phi21 NProtein (SEQ ID NO: 63: N′-GTAKSRYKARRAELIAER-C′), an SV40NLS (PKKKRKV: SEQ ID NO: 3) as a nuclear localization domain and an inter-domain connector (SEQ ID NO: 14: GSLEGGSGG).

The protein moiety in example 11B contains an amino-acid sequence adapted from a FokI nuclease domain as the Functional Domain; an amino-acid sequence adapted from anti-DIG single-chain variable fragment (scFv) immunoglobin (DIG-ScFv) similar to that described in (Huston et. al., 1988) as Linking Domain; an SV40NLS (PKKKRKV: SEQ ID NO: 3) as a nuclear localization domain and an inter-domain connector (SEQ ID NO: 14: GSLEGGSGG).

The nucleic acid end-modifications of the SCNA are NHS-Ester linked Digoxigenin (DIG) and are attached to both the 5′ and the 3′ position of the oligonucleotide.

### Example 11A: Phi21NP Based Programmable Molecular Complex Protein Moiety Sequence

### Components:

Bacteriophage Phi21 NProtein (SEQ ID NO: 63: GTAKSRYKARRAELIAER) at or near the N′ terminus as in the full-length N-protein the RNA-binding peptide is situated at the N-terminus.

FokI Nuclease:

(SEQ ID NO: 66)
VKSELEEKKSELRHKLKYVPHEYIELIEIARNSTQDRILEMKVMEFFMKV
YGYRGKHLGGSRKPDGAIYTVGSPIDYGVIVDTKAYSGGYNLPIGQADEM
QRYVEENQTRNKHINPNEWWKVYPSSVTEFKFLFVSGHFKGNYKAQLTRL
NHITNCNGAVLSVEELLIGGEMIKAGTLTLEEVRRKFNNGEINF
SV40-NLS:
(PKKKRKV: SEQ ID NO: 3)

Interdomain connectors: various poly-amino-acid linkers are tested for optimal function of the programmed molecular complex.
Amino-acid sequence of the protein moiety of the molecular complex: In this example, the Phi21 NProtein (amino acid sequence as set forth in SEQ ID NO:68) is assembled in the N′ terminus of the protein moiety of the programmable molecular construct and the nuclear localization signal, SV40NLS, is located at the C′ terminus and the interdomain linker is

(SEQ ID NO: 7)
GGSGG

### Example 11B: Amino-Acid Sequence (One Letter Code) of the Protein Moiety of the Molecular Complex (NLS-FokI-Nuclease with Digoxygenin ScFv, is Set Forth in SEQ ID NO: 12)

### SCNA Properties and Sequence

The SCNA length of the complementary, target-base-pairing oligonucleotide can be at any predetermined length. For example, the length is at least 18 bases. The SCNA can also contain a small number (preferably 0-6, more preferably 1-2) of non-target-base-pairing nucleotides (N's) of any sequence composition that serve as a spacer between the A) Phi21 boxB RNA hairpin terminal modifier in example 11A or 11B) DIG-NHS terminal-modifier in example 12B, and the complementary nucleotides. In these examples, the SCNAs are connected by a non-target-base pairing sequence designated the “SCNA Connecter” in FIG. 14 or X(n) in the sequences of this example. X(n) signifies an undetermined length of RNA nucleotides connecting the two specificity conferring regions to each other. For linear DNA the expected optimal length (n) is about, 35-73 nucleotides (nts), while both longer (above 73 nts) and shorter (4-34 nts) SCNA connectors are applicable. In the examples given here n=40 nucleotides.

The SCNAs can be complementary to either sense or antisense strands, but are chosen preferably to base-pair with the sense (untranscribed) sequence though two options are shown here for each example. Both SCNA sequences can base-pair with the same strand, as the protein moiety position is situated at the “near end” of the SCNA as defined by the 5′ or 3′ modification of the primer being at the “near end”.

Target “STOP GFP” containing plasmid for the assays of Examples 11A and 11B contains the nucleic acid sequence as set forth in (SEQ ID NO: 181).

### Example 11A: (Phi21NP Based)

Sense or antisense hybridizing dual SCNAs are constructed:

Sense connected SCNAs:
GFP-921SR-X(n)-892SL BOXBPHI
UUCACCUCUAACCGGGUGAGNUCCAAGGGCGAGGAGCUGUUCA (SEQ
ID NO: 207)-X(n)-ACCAUUUACGAACGAUAGCCAUNUUCACCU
CUAACCGGGUGAG(designated as SEQ ID NO: 208).
Anti-sense connected SCNAs:
GFP-921ASR-X(n)-892ASL BOXBPHI
UUCACCUCUAACCGGGUGAGNAUGGCUAUCGUUCGUAAAUGGU (SEQ
ID NO: 209)-X(n)-UGAACAGCUCCUCGCCCUUGGANUUCACC
UCUAACCGGGUGAG (SEQ ID NO: 210)

The 20-mer boxB PHI sequence 5′-UUCACCUCUAACCGGGUGAG-3′ (SEQ ID NO: 62) is underlined. Specificity-conferring sequences on the dual SCNA are marked in the schematic drawings of FIGS. 4A-B as SCNA1 and SCNA2. N's signify a short stretch (0-6) of any nucleotide, X(n) signifies a non-target hybridizing stretch of nucleotides of undefined sequence or length (SCNA Connector).

### Example 11B

Sense or antisense hybridizing dual SCNAs are constructed:

Sense connected SCNAs:
GFP-919SR-X(n)-894SL-DIG
/5DigN/NNTGTCCAAGGGCGAGGAGCTGTT (the nucleic acids
only are designated as SEQ ID NO: 211)
-X(n)- CATTTACGAACGATAGCCATGGNN/3DigN/ (the nucleic
acids only are designated as SEQ ID NO: 212)
Antisense connected SCNAs:
GFP-919ASR-X(n)-894ASL-DIG
/5DigN/NNCCATGGCTATCGTTCGTAAATG (the nucleic acids
only are designated as
SEQ ID NO: 213)-X(n)-AACAGCTCCTCGCCCTTGGACANN/
3DigN/(the nucleic acids only are designated as
SEQ ID NO: 214)

Modification symbols are those used in the Integrated DNA Technology (IDT) website (5′ DIG=/5DigN/; 3′DIG=/3DigN/), X(n) signifies a non-target hybridizing stretch of nucleotides of undefined sequence or length (SCNA Connector).

### Delivery

Bioassay setup: Arabidopsis protoplast preparation is based on Wu et. al. (Wu et. al., 2009) and is similar to that of example 1 with differences in the transfection step:

### Transfection:

1. Make fresh PEG sol for transfection in 2 ml tube
2. Pour off BSA from 6-well plates and dry
3. Mix ˜5×10⁴ protoplasts (2×10⁴-1×10⁵) in 0.2 ml MMg with a mixture of plasmid comprising the Target plasmid DNA and the Protein Moiety expressing DNA and the dual-SCNA expressing plasmid (For example 12A) or dual SCNA containing ssDNA (For example 12B) of 30-40 μg at RT° in 15 ml round-bottom (snap-cap) tubes.
4. Add equal volume (0.2 ml protoplasts+midiprep vol.) of fresh PEG sol
5. Incubate RT° 5 min
6. Wash by slowly adding 3 ml W5, 1 ml at a time, and mixing
7. Centrifuge 100×g in swing-out 1 min
8. Repeat wash and pellet
9. Resuspend in 1 ml W5 solution
10. Pour into BSA-coated plates
11. Grow protoplasts under 16 hr day optimal light (150 μE·m−2·s−1) @ 22° C., replacing media as needed.
   Protoplasts are then subjected to FACS or DNA extraction as described below.

### Results: Point Mutation by Induced DSB.

In this example, cleavage of the target by the molecular complex results in a Double-Strand-Break (DSB) in the plasmid DNA target. This DSB is created in the STOP codon site, which is digested and is repaired by endogenous NHEJ repair mechanism. NHEJ is prone to mutations, and some of these mutations may abolish the STOP codon and restore an open reading frame resulting in an active GFP open reading frame (ORF). GFP is then detected by means of microscopy or flow cytometer (FACS), enabling the measurement of system efficiency and comparison between variables for its improvement.

### Analysis:

The gene targeting efficiency is determined as the percentage of positive GFP cells. Protoplasts suspended in W5 solution are screened for GFP activity 3 days after transfection using an automated flow-cytometer (FACS). GFP is detected by excitation at 488 nm with emission detected by 530/30 filter. Threshold and compensation factors are set to exclude any false positives.

The target sequence is a STOP codon coupled with a diagnostic restriction site (SpeI ACTAGT, STOP underlined) in the GFP coding sequence. When successfully targeted, the STOP codon and diagnostic restriction site are abolished by a deletion, an insertion or a point mutation event. Repair in a specific frame can also restore GFP expression. The assay is analyzed by FACS as described herein below or by purifying plasmid DNA from the protoplasts using a plasmid miniprep kit (Bioneer K3030) as following: protoplasts in W5 solution are precipitated, and lysed by addition of 250 ul Buffer 1 and proceeding with the protocol as for bacterial pellets in the manufacturer's instructions. The region between the SCNAs is amplified from resulting plasmid preparation by PCR. PCR products are exhaustively cleaved with SpeI. After electrophoresis uncleaved products are excised from the gel, cloned into a T/A cloning vector (pUC57/T Fermentas) and individual clones are sequenced to detect different mutation events.

### Example 12. Bioassay for Determination of Optimal SCNA Distances

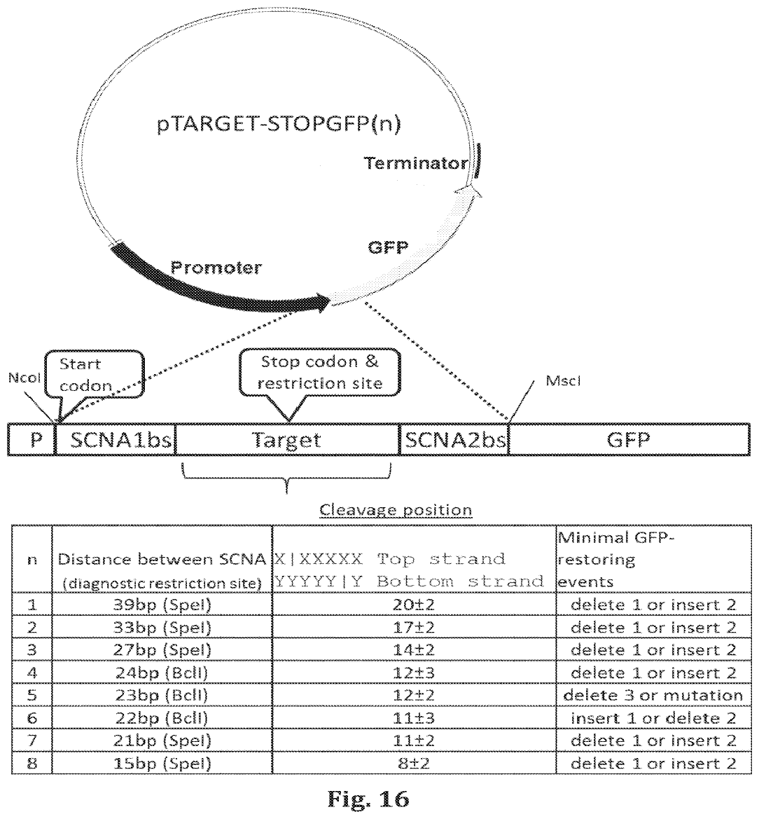

To determine optimal SCNA distances from potential target sites, for each different target type or programmable molecular complex type, a set of target plasmids (pTARGET-STOPGFP(n), FIG. 16.) containing a disrupted GFP reporter coding sequence (STOP-GFP) are created. In an artificial N′ leader and in the GFP coding sequence (CDS) two SCNA binding sequences (SCNAbs) are inserted, which flank a target sequence with variable lengths forming series of plasmids designated as pTARGET-STOPGFP(1-8) (FIG. 16). Inserts, as outlined in FIG. 16 are inserted using the restriction enzymes NcoI and MscI. The target sequence is a STOP codon coupled with a diagnostic restriction site (SpeI ACTAGT (SEQ ID NO:215) or BclI TGATCA (SEQ ID NO:216), STOP underlined) in the artificial N′ leader.

Other components of the plasmid include 1) a promoter operably linked to the GFP sequence. Assay can be conducted in different Eukaryotes. In this example, a plant promoter such as NosP is used for conducting the experiment in Arabidopsis protoplasts. 2) a pair of SCNA binding sites (SCNAlbs and SCNA2bs); 3) a Target site containing a STOP codon; 4) a GFP coding sequence and 5) a transcription terminator sequence, in this example NosT.

The schematic cartoon (not to scale) shown in FIG. 16, illustrates a set of eight exemplary constructs in a set of plasmids pTarget-STOPGFP(n), containing a disrupted Green Fluorescent Protein (GFP) reporter coding sequence (STOPGFP), where “n” signifies a serial number as shown in the table in FIG. 16. The set of inserts of variable length and composition are delineated by an NcoI restriction site encompassing the start codon and an MscI site at the opposite end. SCNAlbs is located in the GFP-artificial N′ leader and SCNA2bs is located in the GFP coding sequence. The target sequence is a STOP codon coupled with a diagnostic restriction site (SpeI ACTAGT (SEQ ID NO:215) or BclI TGATCA (SEQ ID NO:216), STOP underlined) and a frameshift (except in n=5) in the artificial N′ leader. Sequences of the target site spacers are shown in Example 12. In the table, “n” signifies the plasmid serial number. The distance between SCNAbs in base-pairs (bp) is shown followed by the relevant diagnostic restriction site in parenthesis. Desired cleavage positions on the top and bottom strands, due to expected four bp 5′ overhangs, are shown, where ±2 numbers are in even-numbered inserts and +3 numbers in odd-numbered inserts, due to uncertainty caused by the positioning of the catalytic location “on” a nucleotide instead of between nucleotides. In some cleavage events endogenous repair mechanisms may cause imperfect repair causing the deletion, mutation or addition of non-templated nucleotides. Some of these repaired sequences may cause the abolishment of the STOP codon and of the diagnostic restriction site coupled with a frame shift restoring GFP expression. The minimal restoring events, addition or deletion of nucleotides or point mutations, are shown in the rightmost column of the table.

Recognition sequence of SCNA1 binding in insert:
(SEQ ID NO: 182)
ATCTCAAGTCTCTAGGACTGGT
Recognition sequence of SCNA2 binding in GFP
sequence:
(SEQ ID NO: 183)
ATCTGTGAGCAAAGGCGAGGAG
As outlined in FIG. 16:
NcoI/MscI insert for n = 1:
(SEQ ID NO: 184)
CCATGGGATCTCAAGTCTCTAGGACTGGTCTTCAAAATCTTTCTCACT
AGTTTCTACGATCTTGGCCA
NcoI/MscI insert for n = 2:
(SEQ ID NO: 185)
CCATGGGATCTCAAGTCTCTAGGACTGGTCAAAATCTTTCTCACTAGT
TTCTACGCTGGCCA
NcoI/MscI insert for n = 3:
(SEQ ID NO: 186)
CCATGGGATCTCAAGTCTCTAGGACTGGTAATCTTTCTCACTAGTTAC
GCTGGCCA
NcoI/MscI insert for n = 4:
(SEQ ID NO: 187)
CCATGGGATCTCAAGTCTCTAGGACTGGTAATCTTTCTTGATCAGTCTG
GCCA
NcoI/MscI insert for n = 5:
(SEQ ID NO: 188)
CCATGGGATCTCAAGTCTCTAGGACTGGTAATCTTTCTTGATCAGCTGG
CCA
NcoI/MscI insert for n = 6:
(SEQ ID NO: 189)
CCATGGGATCTCAAGTCTCTAGGACTGGTAATCTTTCTTGATCACTGGC
CA
NcoI/MscI insert for n = 7:
(SEQ ID NO: 190)
CCATGGGATCTCAAGTCTCTAGGACTGGTCTTTCTCACTAGTTCTGGCCA
NcoI/MscI insert for n = 8:
(SEQ ID NO: 191)
CCATGGGATCTCAAGTCTCTAGGACTGGTCTTCACTAGTGGCCA

Each molecular complex is co transfected into Arabidopsis protoplasts as described herein below:

### Delivery

Bioassay setup: Arabidopsis protoplast preparation is based on (Wu et. al.) and is similar to that of example 1 with differences in the transfection step:

### Transfection:

1. Make fresh PEG sol for transfection in 2 ml tube
2. Pour off BSA from 6-well plates and dry
3. Mix ˜5×10\hat{ }4 protoplasts (2×10\hat{ }4-1×10\hat{ }5) in 0.2 ml MMg with a mixture of Donor plasmid DNA (where relevant), Protein Moiety expressing plasmid DNA and SCNAs ssDNA to a total of 30-40 microgram at RT° in 15 ml round-bottom (snap-cap) tubes. Alternatively Donor DNA and Protein-moiety expressing DNA are constructed and delivered on a single plasmid.
4. Add equal volume (0.2 ml protoplasts+midiprep vol.) of fresh PEG sol
5. Incubate RT° 5 min
6. Wash by slowly adding 3 ml W5, 1 ml at a time, and mixing
7. Centrifuge 100×g in swing-out 1 min
8. Repeat wash and pellet
9. Resuspend in 1 ml W5 solution
10. Pour into BSA-coated plates
11. Grow protoplasts under 16 hr day optimal light (150 microEinstein·m\hat{ }−2·s\hat{ }−1) at 22 degrees C., replacing media as needed.

### Analysis:

The gene targeting efficiency of each form of the molecular complex is tested on the pTARGET-STOPGFP(n) plasmid series.

When successfully targeted, the STOP codon and diagnostic restriction site are abolished by a deletion, an insertion or a point mutation event (FIG. 16). Repair in a specific frame can also restore GFP expression (FIG. 16). The assay is analyzed by FACS or by purifying plasmid DNA from the protoplasts using a plasmid miniprep kit (Bioneer K3030) as following: protoplasts in W5 solution are precipitated, and lysed by addition of 250 ul Buffer 1 and proceeding with the protocol as for bacterial pellets in the manufacturer's instructions. The “spacer” region is amplified from resulting plasmid preparation by PCR. PCR products are exhaustively cleaved with SpeI (37° C.) or BclI (50° C.) as appropriate. After electrophoresis, uncleaved products are excised from the gel, cloned into a T/A cloning vector (pUC57/T Fermentas) and individual clones are sequenced to detect different mutation events.

The gene targeting efficiency is then determined as the percentage of positive GFP cells. Protoplasts suspended in W5 solution are screened for GFP activity 3 days after transfection using an automated flow-cytometer (FACS). GFP is detected by excitation at 488 nm with emission detected by 530/30 filter. Threshold and compensation factors are set to exclude any false positives.

Controls included in the experiment are 1) use of illegitimate (non-base pairing) SCNAs to control for non-specific cleavage, 2) use of a pTARGET-STOPGFP missing one target binding site to control for non-dimer action, 3) use of pTARGET-GFP, a similar plasmid without the GFP-disrupting STOP codon and having an in-frame GFP as a positive control, 4) use of pTARGET-STOP-I-SceI-GFP, a plasmid similar to pTARGET-STOPGFP but containing an I-SceI restriction site near the GFP-disrupting STOP codon, in conjunction with pSAT4-NLS-I-SceI, a plasmid expressing a nuclear localized I-SceI restriction enzyme in plant cells, as a comparative heterologous system control.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

### REFERENCES

1. Schierling B, Dannemann N, Gabsalilow L, Wende W, Cathomen T, Pingoud A. (2012). A novel zinc-finger nuclease platform with a sequence-specific cleavage module. Nucleic Acids Res. 2012 March;40(6):2623-38.
2. Eisenschmidt K, Lanio T, Simoncsits A, Jeltsch A, Pingoud V, Wende W, Pingoud A. (2005). Developing a programmed restriction endonuclease for highly specific DNA cleavage. Nucleic Acids Res. 2005 Dec. 14; 33(22):7039-47.
3. Kubo T, Kanno K, Ohba H, Rumiana B, Fujii M. (2004). Control of intracellular delivery of oligonucleotides by signal peptides and genetic expression in human cells. Nucleic Acids Symp Ser (Oxf). 2004;(48):303-4.
4. Jinek M, Chylinski K, Fonfara I, Hauer M, Doudna J A, Charpentier E. (2012). A programmable dual-RNA-guided DNA endonuclease in adaptive bacterial immunity. Science. 2012 Aug. 17; 337(6096):816-21.
5. Hanic-Joyce P J, Gray M W (1991) Accurate transcription of a plant mitochondrial gene in vitro. Mol Cell Biol 11: 2035-2039
6. Vainstein A, Marton I, Zuker A, Danziger M, Tzfira T (2011) Permanent genome modifications in plant cells by transient viral vectors. Trends in Biotechnology 29: 363-369
7. Gallois P, Marinho P (1995) Leaf disk transformation using Agrobacterium tumefaciens-expression of heterologous genes in tobacco. Methods Mol Biol 49: 39-48
8. Kochevenko A, Willmitzer L (2003) Chimeric RNA/DNA oligonucleotide-based site-specific modification of the tobacco acetolactate syntase gene. Plant Physiol 132: 174-184
9. Marrs K A, Urioste J C (1995) Transient Gene Expression Analysis in Electroporated Maize Protoplasts. Vol. 55, pp 133-145.
10. Kotlo K U, Yehiely F, Efimova E, Harasty H, Hesabi B, Shchors K, Einat P, Rozen A, Berent E, Deiss L P (2003) Nrf2 is an inhibitor of the Fas pathway as identified by Achilles' Heel Method, a new function-based approach to gene identification in human cells. Oncogene 22: 797-806
11. Huston J S, Levinson D, Mudgett-Hunter M, Tai M S, Novotny J, Margolies M N, Ridge R J, Bruccoleri R E, Haber E, Crea R, et. al. (1988) Protein engineering of antibody binding sites: recovery of specific activity in an anti-digoxin single-chain Fv analogue produced in Escherichia coli. Proc Natl Acad Sci USA 85: 5879-5883
12. Wu F H, Shen S C, Lee L Y, Lee S H, Chan M T, Lin C S (2009) Tape-Arabidopsis Sandwich—a simpler Arabidopsis protoplast isolation method. Plant Methods 5: 16.
13. Antonelli N M, Stadler J (1989) Chemical methods for direct gene transfer to maize protoplasts: I. Efficient transient expression after treatment with the polycation Polybrene Maize News letter 63: 21-22
14. Sheen J (2001) Signal transduction in maize and Arabidopsis mesophyll protoplasts. Plant Physiol 127: 1466-1475
15. Gordon-Kamm W J, Spencer T M, Mangano M L, Adams T R, Dames R J, Start W G, O'Brien J V, Chambers S A, Adams W R, Jr., Willetts N G, Rice T B, Mackey C J, Krueger R W, Kausch A P, Lemaux P G (1990) Transformation of Maize Cells and Regeneration of Fertile Transgenic Plants. Plant Cell 2: 603-618.

1.-38. (canceled)

39. A composition for modifying a predetermined target site in a target nucleic acid sequence in a target cell, the composition comprising:
(a) a programmable polypeptide, or a nucleic acid molecule encoding the programmable polypeptide, the programmable polypeptide comprising:
(i) a functional domain capable of modifying the target site, the functional domain being devoid of a specific nucleic acid binding site; and
(ii) a linking domain that is capable of interacting with a specificity conferring nucleic acid (SCNA) molecule, the linking domain being devoid of a specific target-nucleic acid binding site;
(b) an SCNA molecule, or a nucleic acid molecule encoding the SCNA molecule, the SCNA molecule comprising:
(i) a nucleotide sequence complementary to a region of the target nucleic acid sequence flanking the target site; and
(ii) a recognition region capable of specifically attaching to the linking domain of the programmable polypeptide;
wherein physical association between the linking domain of the programmable polypeptide and the recognition region of the SCNA molecule forms a programmable nucleoprotein complex, and wherein the programmable nucleoprotein complex is capable of modifying the target site.

40. The composition of claim 39, wherein the SCNA molecule provides the specificity and binding capabilities of the programmable nucleoprotein complex to the target nucleic acid through base-pairing of the SCNA and the target nucleic acid.

41. The composition of claim 39, wherein the modifying is selected from the group consisting of: mutation, deletion, insertion, replacement, binding, digestion, double-strand break creation, nicking, methylation, acetylation, ligation, recombination, helix unwinding, chemical modification, labeling, activation, and inactivation.

42. The composition of claim 39, wherein the programmable polypeptide further comprises a subcellular localization domain.

43. The composition of claim 39, wherein the target nucleic acid molecule is a genomic DNA molecule.

44. The composition of claim 39, wherein the target nucleic acid molecule is an organellar DNA molecule.

45. The composition of claim 39, wherein the linking domain interacts with the SCNA molecule due to one or more of the following types of interactions: ligand-receptor, ligand-substrate, hydrogen bonds, van der Waals bond, covalent bonds formed in vivo, ionic bonds, and hydrophobic interactions.

46. The composition of claim 39, wherein the SCNA molecule comprises a nucleic acid molecule selected from the group consisting of a single-strand DNA, a single strand RNA, a double strand RNA, a modified DNA, a modified RNA, a locked-nucleic acid (LNA) and a peptide-nucleic acid (PNA) or combinations thereof.

47. The composition of claim 39, wherein the interaction between the SCNA molecule and the target nucleic acid is through base pairing, selected from the group consisting of a full double helix base pairing, a partial double helix base pairing, a full triple helix base pairing, a partial triple helix base pairing, D-loops or branched forms, formed by said pairing.

48. The composition of claim 39, wherein the programmable polypeptide comprises an interdomain connector or a spacer between the effector domain and the linking domain.

49. The composition of claim 39, wherein the recognition region comprises a chemical modification, and wherein interaction between the chemical modification and the linking domain results from a non-covalent interaction of a binding pair selected from the group consisting of: Biotin-Avidin; Biotin-Streptavidin; Biotin-modified forms of Avidin; protein-protein interactions; protein-nucleic acid interactions; ligand-receptor interactions; ligand-substrate interactions; antibody-antigen interactions; single chain antibody-antigen; antibody or single chain antibody-hapten interactions; hormone-hormone binding protein; receptor-agonist; receptor-receptor antagonist; anti-Fluorescein single-chain variable fragment antibody (anti-FAM ScFV)—Fluorescein; anti-DIG single-chain variable fragment (scFv) immunoglobin (DIG-ScFv)—Digoxigenin (DIG); IgG—protein A; enzyme-enzyme cofactor; coat protein-nucleic acid and Agrobacterium VirD2—VirD2 binding protein enzyme-enzyme inhibitor; single-strand DNA-VirE2; StickyC—dsDNA; RISC—RNA; viral coat protein-nucleic acid; and Agrobacterium VirD2—VirD2 binding protein.

50. The composition of claim 49, wherein the chemical modification is selected from the group consisting of a 5′-end modification, a 3′-end modification, and an internal modification relative to the recognition region.

51. The composition of claim 39, wherein the recognition region of the SCNA molecule comprises a nucleotide motif capable of specifically attaching to the linking domain of the programmable polypeptide.

52. The composition of claim 39, wherein the recognition region of the SCNA molecule comprises a nucleotide motif that interacts, attaches, or binds to the linking domain, and wherein the interaction is selected from the group consisting of Zinc finger protein—Zinc finger motif, restriction enzyme recognition domain—restriction enzyme recognition sequence; DNA binding domain of transcription factor—DNA motif, repressor—operator; Leucine zipper—promoter; Helix loop helix—E box domain; RNA binding motifs comprising Arginine-Rich Motif domains; αβ protein domains; RNA Recognition Motif (RRM) domains; K-Homology Domains; Double Stranded RNA Binding Motifs; RNA-binding Zinc Fingers; and RNA Targeting Enzymes—cognate specific RNA sequence; HIV-rev protein—Stem IIB of the HIV rev response element (RRE); Bovine immunodeficiency virus (BIV) Tat main binding domain—loop 1 of the BIV trans-acting response element (TAR) sequence; Phage lambda, phi21, and P22 Nproteins—the boxB loop hairpins in the N-utilization (nut) sites in their respective RNA.

53. The composition of claim 39, wherein the recognition domain comprises a nucleotide sequence that is covalently bound to a protein selected from the group consisting of Agrobacterium VirD2 protein, Picornavirus VPg, Topoisomerase, PhiX174 phage A protein, and PhiX A* protein.

54. The composition of claim 39, wherein the functional domain comprises a catalytic domain.

55. The composition of claim 54, wherein the catalytic domain is selected from the group consisting of a DNA binding protein, a nuclease, a methylase, a methyltransferase, a methylated DNA binding factor, a transcription factor, a chromatin remodeling factor, a polymerase, a demethylase, an acetylase, a deacetylase, a kinase, a phosphatase, an integrase, a recombinase, a ligase, a topoisomerase, a gyrase, and a helicase.

56. A host cell comprising the composition of claim 39.

57. The host cell of claim 56, wherein the host cell is a eukaryotic cell.

58. The host cell of claim 56, wherein the host cell is a plant cell.

59. The host cell of claim 56, wherein the host cell is an animal cell.


SEQUENCE LISTING (217 sequences)

SEQ ID NO: 1 - DNA, 13 bp, synthetic construct
  misc_feature 1..13: note = polynucleotide
  source 1..13: mol_type = other DNA
ctctgtatcttgt

SEQ ID NO: 2 - DNA, 25 bp, synthetic construct
  misc_feature 1..25: note = polynucleotide
  source 1..25: mol_type = other DNA
gtttacccgccaatatatcctgtca

SEQ ID NO: 3 - protein, 7 aa, synthetic construct
  REGION 1..7: note = peptide
PKKKRKV

SEQ ID NO: 4 - protein, 25 aa, synthetic construct
  REGION 1..25: note = Peptide
MLSLRQSIRFFKPATRTLCSSRYLL

SEQ ID NO: 5 - protein, 33 aa, synthetic construct
  REGION 1..33: note = polypeptide
MLSALARPASAALRRSFSTSAQNNAKVAVLGAS

SEQ ID NO: 6 - protein, 23 aa, synthetic construct
  REGION 1..23: note = polypeptide
MHSRSALLYRFLRPASRCFSSSS

SEQ ID NO: 7 - protein, 5 aa, synthetic construct
  REGION 1..5: note = peptide
GGSGG

SEQ ID NO: 8 - protein, 12 aa, synthetic construct
  REGION 1..12: note = peptide
NIHHVTWHMDFP

SEQ ID NO: 9 - protein, 7 aa, synthetic construct
  REGION 1..7: note = peptide
PNSLIVP

SEQ ID NO: 10 - protein, 6 aa, synthetic construct
  REGION 1..6: note = peptide
TGLDSP

SEQ ID NO: 11 - protein, 4 aa, synthetic construct
  REGION 1..4: note = peptide
GSLE

SEQ ID NO: 12 - protein, 461 aa, synthetic construct
  REGION 1..461: note = Artificial peptide
MPKKKRKVVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSLEGGSGGFDPEVQLQQSGPELVKPGASVRMSCKSSGYIFTDFYMNWVRQSHGKSLDYIGYISPYSGVTGYNQKFKGKATLTVDKSSSTAYMELRSLTSEDSAVYYCAGSSGNKWAMDYWGHGASVTVSSGGGGSGGGGSGGGGSDVVMTQTPLSLPVSLGDQASISCRSSQSLVHSNGNTYLNWYLQKAGQSPKLLIYKVSNRFSGVPDRFSGSGSGTDFTLKISRVEAEDLGIYFCSQTTHVPPTFGGGTKLEIKR

SEQ ID NO: 13 - DNA, 1386 bp, synthetic construct
  misc_feature 1..1386: note = Polynucleotide
  source 1..1386: mol_type = other DNA
atgcccaaaaagaaacgtaaagttgtgaaatcggaactcgaagagaagaaatccgagctcaggcataagttgaagtatgttccgcacgagtacattgaactaattgagatcgctcgtaactctacacaagaccgtattcttgaaatgaaggtgatggagtttttcatgaaggtttatggatatagagggaagcacttgggtggttctcgaaaacctgatggcgcaatctacacggttggctcgccaattgattatggggtgatagtggatactaaggcatactctggtggatacaatcttcctataggtcaagccgacgaaatgcagaggtatgttgaagaaaatcagacaagaaacaagcatatcaatcccaatgagtggtggaaggtttaccctagctctgtgaccgaattcaaattcttatttgtctccggtcactttaagggaaactataaggctcaattgacaagacttaatcatattacaaactgtaatggtgcagtgctcagtgtcgaagagttgcttataggtggggagatgattaaagccggaacgcttaccttggaagaagtacgtagaaaattcaataacggggagataaactttggaagcttagagggtggaagtggtggcttcgatccggaagtccagctccaacaaagtggacctgaactagttaagcctggcgcatccgttagaatgtcttgtaaatcaagtggatacatctttacagatttctacatgaactgggttaggcaatctcatgggaagtctcttgattacattggctatatctctccatattctggtgtaactgggtacaatcagaagtttaaaggtaaggctactttaactgtcgataaatcatcttctacagcgtatatggagttaagatcactgacatccgaagattcagctgtgtattactgtgctgggagttctgggaataagtgggcgatggactattggggccatggtgcttcggtcaccgtatcaagtggcggtggcggatccggtggtggtggctcgggtggtggcggaagtgacgttgtaatgacacaaaccccactgtctcttcctgtcagcctaggtgatcaggctagcatttcttgccgatcctcacaatctttagtgcactcgaatggaaacacttatcttaactggtacttgcagaaggcaggacagtcaccaaaactcttgatctataaagtttcaaacagattctcaggagttcctgacaggtttagcggaagcggatcgggaacagatttcacgctgaaaatctcacgcgttgaggctgaggatctcggtatttacttttgcagccaaactactcatgtgccaccgacttttgggggtggaacgaaattggagataaagaggtga

SEQ ID NO: 14 - protein, 9 aa, synthetic construct
  REGION 1..9: note = peptide
GSLEGGSGG

SEQ ID NO: 15 - DNA, 1875 bp, synthetic construct
  misc_feature 1..1875: note = polynucleotide
  source 1..1875: mol_type = other DNA
gacaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagccatggccgtcgacaactagtccagatctgtgtccaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactctaagcttgggtctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgc

SEQ ID NO: 16 - DNA, 712 bp, synthetic construct
  misc_feature 1..712: note = polynucleotide
  source 1..712: mol_type = other DNA
atggtgagcaagggcgaggaggataacatggccatcatcaaggagttcatgcccttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagataa

SEQ ID NO: 17 - DNA, 21 bp, synthetic construct
  misc_feature 1..21: note = polynucleotide
  source 1..21: mol_type = other DNA
gtcgacaactagtccagatct

SEQ ID NO: 18 - DNA, 28 bp, synthetic construct
  misc_feature 1..28: note = polynucleotide
  variation 1..6: note = n is a, c, g, t or u
  source 1..28: mol_type = other DNA
nnnnnngtgtccaagggcgaggagctgt

SEQ ID NO: 19 - DNA, 28 bp, synthetic construct
  misc_feature 1..28: note = polynucleotide
  variation 23..28: note = n is a, c, g, t or u
  source 1..28: mol_type = other DNA
tttacgaacgatagccatggccnnnnnn

SEQ ID NO: 20 - DNA, 24 bp, synthetic construct
  misc_feature 1..24: note = polynucleotide
  variation 1..2: note = n is a, c, g, t or u
  source 1..24: mol_type = other DNA
nngtccaagggcgaggagctgttc

SEQ ID NO: 21 - DNA, 24 bp, synthetic construct
  misc_feature 1..24: note = polynucleotide
  variation 23..24: note = n is a, c, g, t or u
  source 1..24: mol_type = other DNA
atttacgaacgatagccatggcnn

SEQ ID NO: 22 - DNA, 27 bp, synthetic construct
  misc_feature 1..27: note = polynucleotide
  variation 22..27: note = n is a, c, g, t or u
  source 1..27: mol_type = other DNA
cagctcctcgcccttggagacnnnnnn

SEQ ID NO: 23 - DNA, 27 bp, synthetic construct
  misc_feature 1..27: note = polynucleotide
  variation 1..6: note = n is a, c, g, t or u
  source 1..27: mol_type = other DNA
nnnnnnggccatggctatcgttcgtaa

SEQ ID NO: 24 - DNA, 24 bp, synthetic construct
  misc_feature 1..24: note = polynucleotide
  variation 23..24: note = n is a, c, g, t or u
  source 1..24: mol_type = other DNA
gaacagctcctcgcccttggacnn

SEQ ID NO: 25 - DNA, 24 bp, synthetic construct
  misc_feature 1..24: note = polynucleotide
  variation 1..2: note = n is a, c, g, t or u
  source 1..24: mol_type = other DNA
nngccatggctatcgttcgtaaat

SEQ ID NO: 26 - DNA, 21 bp, synthetic construct
  misc_feature 1..21: note = polynucleotide
  source 1..21: mol_type = other DNA
gactctaagcttgggtctaga

SEQ ID NO: 27 - DNA, 24 bp, synthetic construct
  misc_feature 1..24: note = polynucleotide
  variation 1..2: note = n is a, c, g, or t
  source 1..24: mol_type = other DNA
nntccgcaaaaatcaccagtctct

SEQ ID NO: 28 - DNA, 24 bp, synthetic construct
  misc_feature 1..24: note = polynucleotide
  variation 23..24: note = n is a, c, g, or t
  source 1..24: mol_type = other DNA
gcatggacgagctgtacaagtcnn

SEQ ID NO: 29 - DNA, 24 bp, synthetic construct
  misc_feature 1..24: note = polynucleotide
  variation 23..24: note = n is a, c, g, or t
  source 1..24: mol_type = other DNA
agagactggtgatttttgcggann

SEQ ID NO: 30 - DNA, 24 bp, synthetic construct
  misc_feature 1..24: note = polynucleotide
  variation 1..2: note = n is a, c, g, or t
  source 1..24: mol_type = other DNA
nngacttgtacagctcgtccatgc

SEQ ID NO: 31 - DNA, 22 bp, synthetic construct
  misc_feature 1..22: note = polynucleotide
  source 1..22: mol_type = other DNA
ctatccttcgcaagacccttcc

SEQ ID NO: 32 - DNA, 22 bp, synthetic construct
  misc_feature 1..22: note = polynucleotide
  source 1..22: mol_type = other DNA
ttatcttgtacagctcgtccat

SEQ ID NO: 33 - DNA, 22 bp, synthetic construct
  misc_feature 1..22: note = polynucleotide
  source 1..22: mol_type = other DNA
ccctataagaaccctaattccc

SEQ ID NO: 34 - DNA, 20 bp, synthetic construct
  misc_feature 1..20: note = polynucleotide
  source 1..20: mol_type = other DNA
atggtgagcaagggcgagga

SEQ ID NO: 35 - DNA, 21 bp, synthetic construct
  misc_feature 1..21: note = polynucleotide
  source 1..21: mol_type = other DNA
ttctcaagtcatgagcaactc

SEQ ID NO: 36 - DNA, 28 bp, synthetic construct
  misc_feature 1..28: note = polynucleotide
  variation 1..6: note = n is a, c, g, or t
  source 1..28: mol_type = other DNA
nnnnnnctgtggggccatatcccagaac

SEQ ID NO: 37 - DNA, 28 bp, synthetic construct
  misc_feature 1..28: note = polynucleotide
  variation 23..28: note = n is a, c, g, or t
  source 1..28: mol_type = other DNA
gcgggcaccgagttgtattgtannnnnn

SEQ ID NO: 38 - DNA, 28 bp, synthetic construct
  misc_feature 1..28: note = polynucleotide
  variation 23..28: note = n is a, c, g, or t
  source 1..28: mol_type = other DNA
gttctgggatatggccccacagnnnnnn

SEQ ID NO: 39 - DNA, 28 bp, synthetic construct
  misc_feature 1..28: note = polynucleotide
  variation 1..6: note = n is a, c, g, or t
  source 1..28: mol_type = other DNA
nnnnnntacaatacaactcggtgcccgc

SEQ ID NO: 40 - DNA, 23 bp, synthetic construct
  misc_feature 1..23: note = polynucleotide
  variation 1..2: note = n is a, c, g, or t
  source 1..23: mol_type = other DNA
nngtggggccatatcccagaact

SEQ ID NO: 41 - DNA, 24 bp, synthetic construct
  misc_feature 1..24: note = polynucleotide
  variation 23..24: note = n is a, c, g, or t
  source 1..24: mol_type = other DNA
agcgggcaccgagttgtattgtnn

SEQ ID NO: 42 - DNA, 24 bp, synthetic construct
  misc_feature 1..24: note = polynucleotide
  variation 1..2: note = n is a, c, g, or t
  source 1..24: mol_type = other DNA
nnacaatacaactcggtgcccgct

SEQ ID NO: 43 - DNA, 23 bp, synthetic construct
  misc_feature 1..23: note = polynucleotide
  variation 22..23: note = n is a, c, g, or t
  source 1..23: mol_type = other DNA
agttctgggatatggccccacnn

SEQ ID NO: 44 - DNA, 22 bp, synthetic construct
  misc_feature 1..22: note = polynucleotide
  source 1..22: mol_type = other DNA
gagctagatagcagatgcagat

SEQ ID NO: 45 - DNA, 22 bp, synthetic construct
  misc_feature 1..22: note = polynucleotide
  source 1..22: mol_type = other DNA
ctccagaaaatccctagaaaca

SEQ ID NO: 46 - DNA, 1988 bp, synthetic construct
  misc_feature 1..1988: note = polynucleotide
  source 1..1988: mol_type = other DNA
ggtgtctgacagcagaagatcctcgatggagatggatggggttctgcaagccgcggatgccaaggactgggtttacaagggggaaggcgccgcgaatctcatcctcagctacaccggctcgtcgccctccatggtaagcgctgagtaggttcttactgagcgtgcacgcatcgatcacttgactttaggggctcaatgtgtgattcacgggtgccgcggcgccattcgagctccagatccagtaccgctcgagcaagtgataaaacatggagcagggacgatcacgtggtcacttgaaaattacgtgaggtccggggcgacgatgtacggcgccggcgaactctcaaacactcacacaaccaaaaccgcttcgtgttcgtctttgttccaagcgactgtgtgagtgtttgagagttcgccagcgccgtacatcgtcgccccggatctgacaaattaagctttcgttgcttttccatgattgtgcattttgtgagcatgcactgaatactatgatggatatgtttggaggaagcattattccaatttgatgataagggtgttatttacacttgttttcagcttggcaaggtactgcggctcaagaagattctaaaaaacaagatgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactcaagcctatgcagtgcatgttatgagccaacacctgggtgccaatcatgtcgatggtggggtatggttcagattcagttcatttatgtcctgttattgtgattttgattggtaacatattgacaacctcgacacttgggatcagattcagttcacttatggaagaaattggagaattgttataatttatctataatcacccctactgaaatagaaataacatggcatcaatgtgcatgctattggattttgacacgaatatgctttattctatcatatgttggtaattccagcaggcagcaggcactactctttggatccacgtgacttgacaaagaaatcatgccatctttccacaatgcaggtccgtgtacgtgtttctagggattttctggagcttgtcgaaaagaatgttcttagcagccgtcctgctgggagagtaaatgcaagttcaattgataacactgctgatgccgctcttctaatagcagaccactctttattttctggtacgtactctatccctcttcttaccataatctgaatcttgttaaggtttaaaatatatgattgattaagtaaaatccagagctctattcatatctcatgcactgatgttttgatgaaacacttgtagcaagacggttgc

SEQ ID NO: 47 - DNA, 2987 bp, synthetic construct
  misc_feature 1..2987: note = polynucleotide
  source 1..2987: mol_type = other DNA
ggtgtctgacagcagaagatcctcgatggagatggatggggttctgcaagccgcggatgccaaggactgggtttacaagggggaaggcgccgcgaatctcatcctcagctacaccggctcgtcgccctccatggtaagcgctgagtaggttcttactgagcgtgcacgcatcgatcacttgactttaggggctcaatgtgtgattcacgggtgccgcggcgccattcgagctccagatccagtaccgctcgagcaagtgataaaacatggagcagggacgatcacgtggtcacttgaaaattacgtgaggtccggggcgacgatgtacggcgccggcgaactctcaaacactcacacaaccaaaaccgcttcgtgttcgtctttgttccaagcgactgtgtgagtgtttgagagttcgccagcgccgtacatcgtcgccccggatctgacaaattaagctttcgttgcttttccatgattgtgcattttgtgagcatgcactgaatactatgatggatatgtttggaggaagcattattccaatttgatgataagggtgttatttacacttgttttcagcttggcaaggtactgcggctcaagaagattctaaaaaacaagtaaatagtataaccggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagcatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggctcaagcctatgcagtgcatgttatgagccaacacctgggtgccaatcatgtcgatggtggggtatggttcagattcagttcatttatgtcctgttattgtgattttgattggtaacatattgacaacctcgacacttgggatcagattcagttcacttatggaagaaattggagaattgttataatttatctataatcacccctactgaaatagaaataacatggcatcaatgtgcatgctattggattttgacacgaatatgctttattctatcatatgttggtaattccagcaggcagcaggcactactctttggatccacgtgacttgacaaagaaatcatgccatctttccacaatgcaggtccgtgtacgtgtttctagggattttctggagcttgtcgaaaagaatgttcttagcagccgtcctgctgggagagtaaatgcaagttcaattgataacactgctgatgccgctcttctaatagcagaccactctttattttctggtacgtactctatccctcttcttaccataatctgaatcttgttaaggtttaaaatatatgattgattaagtaaaatccagagctctattcatatctcatgcactgatgttttgatgaaacacttgtagcaagacggttgc

SEQ ID NO: 48 - DNA, 1672 bp, synthetic construct
  misc_feature 1..1672: note = polynucleotide
  source 1..1672: mol_type = other DNA
atggtcatttcttctggttcaagcatgacatgaacaggcaataaataagttgagattttgatcacagtaactgatacttgaatcgaatcatttagattttttttttttttagtttacttgtttagtaaatatgttgtctatgtttgtcacaaaaacgtggctcagttcttgtatatatggagacaaaaaaatccattaaaagattgttgacattctcggaaatttagtgccaactgttattgcgagaacttactatagttttcctttggcgaaaagctaataatcttaaatcttgattttgtcctcttttctctgagttagattttcttaaattccacttccgacctattaagaaatgggcttttgcaaagaagatccgcttcactgagcccgtatctcgaagaggataatacaacaacaaagcaaaacggcacgtagttttaattgtaaccaaggattgcatttcggtcttgtttcaacaaacgaaacttcctgaaatgccaagaaaaatctggtcatttcaccacagtgatcattgtgtatgtgttctaaagactccaagcgaaggttttagaaaaaggagcattttctattctattcaagaaactcgaagaacattctctcttcatcctctaacttccctataaatatgtcctttgctaatcagatcaaatcagcaggaaaatcaagaaccaaaagtctcccgaaaagcaacgaacaatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggatcccccgggctgcaggaattcgatccgctagcatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctagcgg

SEQ ID NO: 49 - protein, 462 aa, synthetic construct
  REGION 1..462: note = polypeptide
MPKKKRKVVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGGSGGSDVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKSSADDAKKDAAKKDDAKKDDAKKDGGVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVS

SEQ ID NO: 50 - DNA, 1389 bp, synthetic construct
  misc_feature 1..1389: note = polynucleotide
  source 1..1389: mol_type = other DNA
atgccgaaaaagaagaggaaggtggtgaagagcgaactggaggagaaaaagtccgagctccgccacaagctgaagtacgtcccgcacgagtacatcgaactgatcgagattgccaggaattccacccaggaccgcatcctcgaaatgaaggtcatggaattcttcatgaaggtgtacggctaccgcgggaaacacctcggcggcagcaggaagccagatggcgcgatctatacggtcgggtccccgatcgactatggggtgatcgtcgacaccaaagcgtacagcggcggctacaacctcccaattggccaggcggacgagatgcagaggtatgtggaggagaaccagacccgcaataagcacatcaacccgaacgagtggtggaaggtgtacccaagctccgtgaccgagttcaagttcctcttcgtgtccgggcacttcaagggcaattacaaggcccagctgacgcgcctcaaccatatcacgaattgcaatggcgccgtcctgtccgtcgaagagctgctcatcggcggggagatgatcaaggcgggcacgctgacgctggaggaagtacgtaggaaattcaacaacggggaaatcaatttcgggggatccgggggcagcgatgtggtgatgacgcagaccccactgtccctgccagtgtccctaggcgaccaagcgagcatctcctgtcgcagcagccagtccctcgtgcactccaacggcaacacgtatctccgctggtatctccagaagccggggcagtccccgaaagtgctgatctacaaggtctccaaccgcttctccggcgtcccggataggtttagcggcagcgggtccggcaccgactttaccctgaaaatctccagggtggaggccgaggatctcggggtctacttttgcagccagagcacgcacgtcccatggacctttgggggcggcacgaaactggagattaaaagcagcgctgacgatgccaagaaggacgcggcgaaaaaggacgacgccaagaaagacgacgcgaaaaaagacggcggcgtcaagctcgatgaaaccggcggcgggctcgtgcaaccaggccgcccgatgaagcttagctgcgtggccagcgggttcaccttcagcgactactggatgaactgggtccgccaatccccggaaaagggcctggagtgggtggcccagatcaggaacaagccatataactatgagacgtactactccgatagcgtgaaagggcgcttcaccatctccagggacgacagcaagtcctccgtgtacctccagatgaacaacctacgcgtggaggatatgggcatctactactgcaccgggagctactacggcatggactactggggccagggcacgtccgtcaccgtgtcctga

SEQ ID NO: 51 - DNA, 21 bp, synthetic construct
  misc_feature 1..21: note = polynucleotide
  source 1..21: mol_type = other DNA
gtcctgctaagcctttgaaag

SEQ ID NO: 52 - DNA, 28 bp, synthetic construct
  misc_feature 1..28: note = polynucleotide
  variation 23..28: note = n is a, c, g, or t
  source 1..28: mol_type = other DNA
gcatccttccgtagtgctcctcnnnnnn

SEQ ID NO: 53 - DNA, 28 bp, synthetic construct
  misc_feature 1..28: note = polynucleotide
  variation 1..6: note = n is a, c, g, or t
  source 1..28: mol_type = other DNA
nnnnnnttgtaattgctggtgctggtat

SEQ ID NO: 54 - DNA, 28 bp, synthetic construct
  misc_feature 1..28: note = polynucleotide
  variation 1..6: note = n is a, c, g, or t
  source 1..28: mol_type = other DNA
nnnnnngaggagcactacggaaggatgc

SEQ ID NO: 55 - DNA, 24 bp, synthetic construct
  misc_feature 1..24: note = polynucleotide
  variation 23..24: note = n is a, c, g, or t
  source 1..24: mol_type = other DNA
tgcatccttccgtagtgctcctnn

SEQ ID NO: 56 - DNA, 24 bp, synthetic construct
  misc_feature 1..24: note = polynucleotide
  variation 1..2: note = n is a, c, g, or t
  source 1..24: mol_type = other DNA
nngtaattgctggtgctggtatgt

SEQ ID NO: 57 - DNA, 24 bp, synthetic construct
  misc_feature 1..24: note = polynucleotide
  variation 1..2: note = n is a, c, g, or t
  source 1..24: mol_type = other DNA
nnaggagcactacggaaggatgca

SEQ ID NO: 58 - DNA, 24 bp, synthetic construct
  misc_feature 1..24: note = polynucleotide
  variation 23..24: note = n is a, c, g, or t
  source 1..24: mol_type = other DNA
acataccagcaccagcaattacnn

SEQ ID NO: 59 - DNA, 1865 bp, synthetic construct
  misc_feature 1..1865: note = polynucleotide
  source 1..1865: mol_type = other DNA
aaattacgttgagatgcatggtctctctctactcaattaaccaaataaggaaaagaatcatatggtcatcaattcgtaaatcaaaattttaatttgtgtggtatttaatccatctacatgtttcgtaagcaacaaaagagcttggtctgaaaaccaaacaagaccatatgggcactcgaatactccattttgttatcggctacttccactagcctcctccttcgctgcgtctcctgtttctctacttcacgattactcgctaggtaaaagataagatactaaacaactactatagcccctttaattcaagtccgtttttttttgttcttcttcttttcaattatgtgttaaagatacaaacttttgtctgatttgcttccaccggtttcacctaagatactcaattttcttactttttgtgtgttttgtaattctaattcttttatagcttcaatttttagattcattgaagcagttgtgagttaagttggagaaaatggttgtgtttgggaatgtttctgcggcgaatttgccttatcaaaacgggtttttggaggcactttcatctggaggttgtgaactaatgggacatagctttagggttcccacttctcaagcgcttaagacaagaacaaggaggaggagtactgctggtcctttgatggtgagcaagggcgaggaggataacatggccatcatcaaggagttcatgcccttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaggtatgtctacaacttcatttcttactgctttgtttgggatctgatggaattaatgttcatgtgatgaagttcttttggctctcaaataattgcaggattggctggattgtcaactgcaaagtacctggctgatgcaggccacaaacctctgttgcttgaagcaagagatgttcttggtggaaaggtactttgattggtctcactttccgaattacttattgttgtttgcatttctcattcttatatgatttgctgattcccaaaggctgtaccaattatgtttgtgagactacaaggtataactagtgtttagagtgttacagctcctaggtacccaatgtatctgtcctgtttgttgtattagtcttgtactgcccaatttgttttttattatgttctaatgtcttcagtcttgtgctacacaccgcacaaccctttccagtttctaggcttaatgtgaatcaaatatagagtagtttactggaagatatc

SEQ ID NO: 60 - DNA, 23 bp, synthetic construct
  misc_feature 1..23: note = polynucleotide
  source 1..23: mol_type = other DNA
tggttgtgtttgggaatgtttct

SEQ ID NO: 61 - DNA, 26 bp, synthetic construct
  misc_feature 1..26: note = polynucleotide
  source 1..26: mol_type = other DNA
tatccaaaagatatcttccagtaaac

SEQ ID NO: 62 - RNA, 20 nt, synthetic construct
  misc_feature 1..20: note = polynucleoitde
  source 1..20: mol_type = other RNA
ttcacctctaaccgggtgag

SEQ ID NO: 63 - protein, 18 aa, synthetic construct
  REGION 1..18: note = polypeptide
GTAKSRYKARRAELIAER

SEQ ID NO: 64 - DNA, 21 bp, synthetic construct
  misc_feature 1..21: note = polynucleotide
  source 1..21: mol_type = other DNA
ggtcaagtgccacgtaggatg

SEQ ID NO: 65 - DNA, 21 bp, synthetic construct
  misc_feature 1..21: note = polynucleotide
  source 1..21: mol_type = other DNA
ggtcaagtggcgcgcaggatg

SEQ ID NO: 66 - protein, 194 aa, synthetic construct
  REGION 1..194: note = polypeptide
VKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 67 - protein, 8 aa, synthetic construct
  REGION 1..8: note = peptide
MPKKKRKV

SEQ ID NO: 68 - protein, 225 aa, synthetic construct
  REGION 1..225: note = polypeptide
MGTAKSRYKARRAELIAERGGSGGVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFPKKKRKV

SEQ ID NO: 69 - DNA, 678 bp, synthetic construct
  misc_feature 1..678: note = polypeptide
  source 1..678: mol_type = other DNA
atggggacggccaagtccaggtacaaggcgcgcagggccgaactgattgcggagaggggcggatccggcggcgtcaaatccgagctcgaagagaagaagagcgagctgaggcacaagctgaagtacgtcccgcacgagtacatcgaactcatcgagatcgccaggaactccacccaggaccgcatcctggagatgaaggtgatggagttcttcatgaaggtgtacggctaccgtggcaaacatctggggggctccaggaaaccggatggggccatctacaccgtggggagcccgatcgactacggcgtgatcgtggacaccaaagcgtactccggcggctacaacctcccgattggccaggccgatgaaatgcagcgctacgtggaggagaaccagacgcgcaataagcacatcaacccgaacgagtggtggaaggtgtacccgtcctccgtgaccgagttcaagttcctgttcgtgagcggccacttcaaggggaactacaaggcccagctgacccgcctgaaccacatcaccaactgcaatggggcggtgctctccgtggaggaactcctgatcggcggcgagatgatcaaggccggcaccctgaccctggaagaggtacgtagaaagttcaacaacggcgagatcaacttcccgaagaagaagcgcaaggtgtga

SEQ ID NO: 70 - RNA, 43 nt, synthetic construct
  misc_feature 1..43: note = polynucleotide
  variation 21: note = n is a, c, g, or u
  source 1..43: mol_type = other RNA
ttcacctctaaccgggtgagnggtactgatgcttttcaggaaa

SEQ ID NO: 71 - RNA, 43 nt, synthetic construct
  misc_feature 1..43: note = polynucleotide
  variation 23: note = n is a, c, g, or u
  source 1..43: mol_type = other RNA
atagcgtccccattgttgctatnttcacctctaaccgggtgag

SEQ ID NO: 72 - RNA, 43 nt, synthetic construct
  misc_feature 1..43: note = polynucleotide
  variation 23: note = n is a, c, g, or u
  source 1..43: mol_type = other RNA
tttcctgaaaagcatcagtaccnttcacctctaaccgggtgag

SEQ ID NO: 73 - RNA, 42 nt, synthetic construct
  misc_feature 1..42: note = polynucleotide
  variation 20: note = n is a, c, g, or u
  source 1..42: mol_type = other RNA
tcacctctaaccgggtgagnatagcaacaatggggacgctat

SEQ ID NO: 74 - protein, 8 aa, synthetic construct
  REGION 1..8: note = peptide
GGSGGESK

SEQ ID NO: 75 - protein, 229 aa, synthetic construct
  REGION 1..229: note = polypeptide
MPKKKRKVVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGGSGGESKGTAKSRYKARRAELIAERR

SEQ ID NO: 76 - DNA, 690 bp, synthetic construct
  misc_feature 1..690: note = polypeptide
  source 1..690: mol_type = other DNA
atgccaaagaagaagagaaaggttgttaagtctgaacttgaagaaaagaagtctgaacttagacataagcttaagtatgttccacatgaatatattgaacttattgaaattgctagaaattctactcaagatagaattcttgaaatgaaggttatggaattttttatgaaggtttatggatatagaggaaagcatcttggaggatctagaaagccagatggagctatttatactgttggatctccaattgattatggagttattgttgatactaaggcttattctggaggatataatcttccaattggacaagctgatgaaatgcaaagatatgttgaagaaaatcaaactagaaataagcatattaatccaaatgaatggtggaaggtttatccatcttctgttactgaatttaagtttctttttgtttctggacattttaagggaaattataaggctcaacttactagacttaatcatattactaattgtaatggagctgttctttctgttgaagaacttcttattggaggagaaatgattaaggctggaactcttactcttgaagaagttagaagaaagtttaataatggagaaattaattttggaggatctggaggagaatctaagggaactgctaagtctagatataaggctagaagagctgaacttattgctgaaagaagataa

SEQ ID NO: 77 - RNA, 42 nt, synthetic construct
  misc_feature 1..42: note = polynucleotide
  source 1..42: mol_type = other RNA
ttcacctctaaccgggtgagtactgatgcttttcaggaaact

SEQ ID NO: 78 - RNA, 42 nt, synthetic construct
  misc_feature 1..42: note = polynucleotide
  source 1..42: mol_type = other RNA
gatagcgtccccattgttgctattcacctctaaccgggtgag

SEQ ID NO: 79 - RNA, 42 nt, synthetic construct
  misc_feature 1..42: note = polynucleotide
  source 1..42: mol_type = other RNA
agtttcctgaaaagcatcagtattcacctctaaccgggtgag

SEQ ID NO: 80 - RNA, 42 nt, synthetic construct
  misc_feature 1..42: note = polynucleotide
  source 1..42: mol_type = other RNA
ttcacctctaaccgggtgagtagcaacaatggggacgctatc

SEQ ID NO: 81 - RNA, 43 nt, synthetic construct
  misc_feature 1..43: note = polynucleotide
  variation 21: note = n is a, c, g, or u
  source 1..43: mol_type = other RNA
ttcacctctaaccgggtgagntcggtactgatgcttttcagga

SEQ ID NO: 82 - RNA, 43 nt, synthetic construct
  misc_feature 1..43: note = polynucleotide
  variation 23: note = n is a, c, g, or u
  source 1..43: mol_type = other RNA
gcgtccccattgttgctataacnttcacctctaaccgggtgag

SEQ ID NO: 83 - RNA, 43 nt, synthetic construct
  misc_feature 1..43: note = polynucleotide
  variation 23: note = n is a, c, g, or u
  source 1..43: mol_type = other RNA
tcctgaaaagcatcagtaccganttcacctctaaccgggtgag

SEQ ID NO: 84 - RNA, 43 nt, synthetic construct
  misc_feature 1..43: note = polynucleotide
  variation 21: note = n is a, c, g, or u
  source 1..43: mol_type = other RNA
ttcacctctaaccgggtgagngttatagcaacaatggggacgc

SEQ ID NO: 85 - DNA, 1995 bp, synthetic construct
  misc_feature 1..1995: note = polynucleotide
  source 1..1995: mol_type = other DNA
atggcggcggctgcggcggctccatctccctctttctccaaaaccctatcgtcctcctcctccaaatcctccaccctcctccctagatccaccttccctttcccccaccacccccacaaaaccaccccaccacccctccacctcacccccacccacattcacagccaacgccgtcgtttcaccatctccaatgtcatttccactacccaaaaagtttccgagacccaaaaagccgaaactttcgtttcccgttttgcccctgacgaacccagaaagggttccgacgttctcgtggaggccctcgaaagagaaggggttacggacgtttttgcgtacccaggcggcgcttccatggagattcaccaagctttgacgcgctcaagcatcatccgcaacgtgctaccacgtcacgagcagggtggtgtcttcgccgctgagggttacgcacgcgccaccggcttccccggcgtttgcattgccacctccggccctggcgccaccaatctcgtcagtggcctcgcggacgccctactggatagcgtccccattgttgctataaccggtcaagtgccacgtaggatgatcggtactgatgcttttcaggaaactccgattgttgaggtaactagatcgattaccaagcataattatctcgttatggacgtagaggatattcctagggttgtacgtgaggcttttttccttgcgagatcgggccggcctggccctgttttgattgatgtacctaaggatattcagcaacaattggtgatacctgactgggatcagccaatgaggttgcctggttacatgtctaggttacctaaattgcccaatgagatgcttttagaacaaattgttaggcttatttctgagtcaaagaagcctgttttgtatgtggggggtgggtgttcgcaatcgagtgaggagttgagacgattcgtggagctcaccggtatccccgtggcaagtactttgatgggtcttggagcttttccaactggggatgagctttccctttcaatgttgggtatgcatggtactgtttatgctaattatgctgtggacagtagtgatttattgctcgcatttggggtgaggtttgatgatagagttactggaaagttagaagcttttgctagccgagcgaaaattgttcacattgatattgattcagctgagattggaaagaacaagcagcctcatgtttccatttgtgcggatatcaagttggcgttacagggtttgaattcgatattggagagtaaggaaggtaaactgaagttggatttttctgcttggaggcaggagttgacggtgcagaaagtgaagtacccgttgaattttaaaacttttggtgatgctattcctccgcaatatgctatccaggttctagatgagttaactaatgggagtgctattataagtaccggtgttgggcagcaccagatgtgggctgctcaatattataagtacagaaagccacgccaatggttgacatctggtggattaggagcgatgggatttggtttgcccgctgctattggtgcggctgttggaagacctgatgaagttgtggttgacattgatggtgatggcagtttcatcatgaatgtgcaggagctagcaactattaaggtggagaatctcccagttaagattatgttactgaataatcaacacttgggaatggtggttcaatgggaggatcggttctataaggctaacagagcacacacatacctggggaatccttctaatgaggcggagatctttcctaatatgttgaaatttgcagaggcttgtggcgtacctgctgcgagagtgacacacagggatgatcttagagcggctattcaaaagatgttagacactcctgggccatacttgttggatgtgattgtacctcatcaggaacatgttctacctatgattcccagtggcggggctttcaaagatgtgatcacagagggtgacgggagaagttcctattga

SEQ ID NO: 86 - DNA, 1995 bp, synthetic construct
  misc_feature 1..1995: note = polynucleotide
  source 1..1995: mol_type = other DNA
atggcggcggctgcggcggctccatctccctctttctccaaaaccctatcgtcctcctcctccaaatcctccaccctcctccctagatccaccttccctttcccccaccacccccacaaaaccaccccaccacccctccacctcacccccacccacattcacagccaacgccgtcgtttcaccatctccaatgtcatttccactacccaaaaagtttccgagacccaaaaagccgaaactttcgtttcccgttttgcccctgacgaacccagaaagggttccgacgttctcgtggaggccctcgaaagagaaggggttacggacgtttttgcgtacccaggcggcgcttccatggagattcaccaagctttgacgcgctcaagcatcatccgcaacgtgctaccacgtcacgagcagggtggtgtcttcgccgctgagggttacgcacgcgccaccggcttccccggcgtttgcattgccacctccggccctggcgccaccaatctcgtcagtggcctcgcggacgccctactggatagcgtcccaatagtagccataactggtcaagtggcgcgcaggatgatcggtaccgatgcttttcaggaaactccgattgttgaggtaactagatcgattaccaagcataattatctcgttatggacgtagaggatattcctagggttgtacgtgaggcttttttccttgcgagatcgggccggcctggccctgttttgattgatgtacctaaggatattcagcaacaattggtgatacctgactgggatcagccaatgaggttgcctggttacatgtctaggttacctaaattgcccaatgagatgcttttagaacaaattgttaggcttatttctgagtcaaagaagcctgttttgtatgtggggggtgggtgttcgcaatcgagtgaggagttgagacgattcgtggagctcaccggtatccccgtggcaagtactttgatgggtcttggagcttttccaactggggatgagctttccctttcaatgttgggtatgcatggtactgtttatgctaattatgctgtggacagtagtgatttattgctcgcatttggggtgaggtttgatgatagagttactggaaagttagaagcttttgctagccgagcgaaaattgttcacattgatattgattcagctgagattggaaagaacaagcagcctcatgtttccatttgtgcggatatcaagttggcgttacagggtttgaattcgatattggagagtaaggaaggtaaactgaagttggatttttctgcttggaggcaggagttgacggtgcagaaagtgaagtacccgttgaattttaaaacttttggtgatgctattcctccgcaatatgctatccaggttctagatgagttaactaatgggagtgctattataagtaccggtgttgggcagcaccagatgtgggctgctcaatattataagtacagaaagccacgccaatggttgacatctggtggattaggagcgatgggatttggtttgcccgctgctattggtgcggctgttggaagacctgatgaagttgtggttgacattgatggtgatggcagtttcatcatgaatgtgcaggagctagcaactattaaggtggagaatctcccagttaagattatgttactgaataatcaacacttgggaatggtggttcaatgggaggatcggttctataaggctaacagagcacacacatacctggggaatccttctaatgaggcggagatctttcctaatatgttgaaatttgcagaggcttgtggcgtacctgctgcgagagtgacacacagggatgatcttagagcggctattcaaaagatgttagacactcctgggccatacttgttggatgtgattgtacctcatcaggaacatgttctacctatgattcccagtggcggggctttcaaagatgtgatcacagagggtgacgggagaagttcctattga

SEQ ID NO: 87 - protein, 286 aa, synthetic construct
  REGION 1..286: note = polypeptide
PAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGALFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 88 - protein, 557 aa, synthetic construct
  REGION 1..557: note = polypeptide
MPKKKRKVDVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKSSADDAKKDAAKKDDAKKDDAKKDGGVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSGSLEGGSGGPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGALFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 89 - DNA, 1674 bp, synthetic construct
  misc_feature 1..1674: note = polynucleotide
  source 1..1674: mol_type = other DNA
atgcctaagaagaagagaaaggttgatgttgttatgactcaaactcctctttctcttcctgtttctcttggagatcaagcttctatttcttgtagatcttctcaatctcttgttcattctaatggaaatacttatcttagatggtatcttcaaaagcctggacaatctcctaaggttcttatttataaggtttctaatagattttctggagttcctgatagattttctggatctggatctggaactgattttactcttaagatttctagagttgaagctgaagatcttggagtttatttttgttctcaatctactcatgttccttggacttttggaggaggaactaagcttgaaattaagtcttctgctgatgatgctaagaaggatgctgctaagaaggatgatgctaagaaggatgatgctaagaaggatggaggagttaagcttgatgaaactggaggaggacttgttcaacctggaagacctatgaagctttcttgtgttgcttctggatttactttttctgattattggatgaattgggttagacaatctcctgaaaagggacttgaatgggttgctcaaattagaaataagccttataattatgaaacttattattctgattctgttaagggaagatttactatttctagagatgattctaagtcttctgtttatcttcaaatgaataatcttagagttgaagatatgggaatttattattgtactggatcttattatggaatggattattggggacaaggaacttctgttactgtttctggatctcttgaaggaggatctggaggacctgctgaaaagagaaagcctattagagttctttctctttttgatggaattgctactggacttcttgttcttaaggatcttggaattcaagttgatagatatattgcttctgaagtttgtgaagattctattactgttggaatggttagacatcaaggaaagattatgtatgttggagatgttagatctgttactcaaaagcatattcaagaatggggaccttttgatcttgttattggaggatctccttgtaatgatctttctattgttaatcctgctagaaagggactttatgaaggaactggagctcttttttttgaattttatagacttcttcatgatgctagacctaaggaaggagatgatagaccttttttttggctttttgaaaatgttgttgctatgggagtttctgataagagagatatttctagatttcttgaatctaatcctgttatgattgatgctaaggaagtttctgctgctcatagagctagatatttttggggaaatcttcctggaatgaatagacctcttgcttctactgttaatgataagcttgaacttcaagaatgtcttgaacatggaagaattgctaagttttctaaggttagaactattactactagatctaattctattaagcaaggaaaggatcaacattttcctgtttttatgaatgaaaaggaagatattctttggtgtactgaaatggaaagagtttttggatttcctgttcattatactgatgtttctaatatgtctagacttgctagacaaagacttcttggaagatcttggtctgttcctgttattagacatctttttgctcctcttaaggaatattttgcttgtgtttaa

SEQ ID NO: 90 - DNA, 1896 bp, synthetic construct
  misc_feature 1..1896: note = polynucleotide
  source 1..1896: mol_type = other DNA
accggtcaacatgtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacgtcgagagttctcaacacaacatatacaaaacaaacgaatctcaagcaatcaagcattctacttctattgcagcaatttaaatcatttcttttaaagcaaaagcaattttctgaaaattttcaccatttacgaacgatagagatctcgagctcaagcttcgaattctgcagtcgacggtaccgcgggcccgggatcctgatggtgagcaagggcgaggaggataacatggccatcatcaaggagttcatgcccttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagagatccacctgatctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacgcggccgc

SEQ ID NO: 91 - DNA, 28 bp, synthetic construct
  misc_feature 1..28: note = polynucleotide
  variation 23..28: note = n is a, c, g, or t
  source 1..28: mol_type = other DNA
tcgagctcaagcttcgaattctnnnnnn

SEQ ID NO: 92 - DNA, 28 bp, synthetic construct
  misc_feature 1..28: note = polynucleotide
  variation 1..6: note = n is a, c, g, or t
  source 1..28: mol_type = other DNA
nnnnnngatggtgagcaagggcgaggag

SEQ ID NO: 93 - protein, 17 aa, synthetic construct
  REGION 1..17: note = peptide
SGPRPRGTRGKGRRIRR

SEQ ID NO: 94 - RNA, 31 nt, synthetic construct
  misc_feature 1..31: note = polynucleotide
  source 1..31: mol_type = other RNA
ttcagctcgtgtagctcattagctccgagct

SEQ ID NO: 95 - DNA, 31 bp, synthetic construct
  misc_feature 1..31: note = polynucleotide
  source 1..31: mol_type = other DNA
ttccatacagtcagtatcaattctggaagaa

SEQ ID NO: 96 - DNA, 22 bp, synthetic construct
  misc_feature 1..22: note = polypeptide
  source 1..22: mol_type = other DNA
atcaattctggaagaatttcca

SEQ ID NO: 97 - DNA, 22 bp, synthetic construct
  misc_feature 1..22: note = polynucleotide
  source 1..22: mol_type = other DNA
tcattacacctgcagctctcat

SEQ ID NO: 98 - DNA, 53 bp, synthetic construct
  misc_feature 1..53: note = polynucleotide
  source 1..53: mol_type = other DNA
ttcagctcgtgtagctcattagctccgagctatcaattctggaagaatttcca

SEQ ID NO: 99 - DNA, 53 bp, synthetic construct
  misc_feature 1..53: note = polynucleotide
  source 1..53: mol_type = other DNA
tcattacacctgcagctctcatttcagctcgtgtagctcattagctccgagct

SEQ ID NO: 100 - RNA, 55 nt, synthetic construct
  misc_feature 1..55: note = polynucleotide
  variation 32..33: note = n is a, c, g, or u
  source 1..55: mol_type = other RNA
ttcagctcgtgtagctcattagctccgagctnngtatcaattctggaagaatttc

SEQ ID NO: 101 - DNA, 56 bp, synthetic construct
  misc_feature 1..56: note = polynucleotide
  variation 24..25: note = n is a, c, g, t or u
  source 1..56: mol_type = other DNA
caaaaagaaggtcttcattacacnnttcagctcgtgtagctcattagctccgagct

SEQ ID NO: 102 - DNA, 32 bp, synthetic construct
  misc_feature 1..32: note = polynucleotide
  source 1..32: mol_type = other DNA
tttccatacagtcagtatcaattctggaagaa

SEQ ID NO: 103 - DNA, 31 bp, synthetic construct
  misc_feature 1..31: note = polynucleotide
  source 1..31: mol_type = other DNA
cagggtggaacaagatggattatcaagtgtc

SEQ ID NO: 104 - DNA, 53 bp, synthetic construct
  misc_feature 1..53: note = polynucleotide
  source 1..53: mol_type = other DNA
ttcagctcgtgtagctcattagctccgagctaagtccaatctatgacatcaat

SEQ ID NO: 105 - DNA, 53 bp, synthetic construct
  misc_feature 1..53: note = polynucleotide
  source 1..53: mol_type = other DNA
aagatcactttttatttatgcattcagctcgtgtagctcattagctccgagct

SEQ ID NO: 106 - DNA, 55 bp, synthetic construct
  misc_feature 1..55: note = polynucleotide
  variation 32..33: note = n is a, c, g, t or u
  source 1..55: mol_type = other DNA
ttcagctcgtgtagctcattagctccgagctnntcaagtccaatctatgacatca

SEQ ID NO: 107 - DNA, 55 bp, synthetic construct
  misc_feature 1..55: note = polynucleotide
  variation 23..24: note = n is a, c, g, t or u
  source 1..55: mol_type = other DNA
gatcactttttatttatgcacannttcagctcgtgtagctcattagctccgagct

SEQ ID NO: 108 - DNA, 31 bp, synthetic construct
  misc_feature 1..31: note = polynucleotide
  source 1..31: mol_type = other DNA
atatctgtgggcttgtgacacggactcaagt

SEQ ID NO: 109 - DNA, 53 bp, synthetic construct
  misc_feature 1..53: note = polynucleotide
  source 1..53: mol_type = other DNA
ttcagctcgtgtagctcattagctccgagctgggctggtgacccagtcagagt

SEQ ID NO: 110 - DNA, 53 bp, synthetic construct
  misc_feature 1..53: note = polynucleotide
  source 1..53: mol_type = other DNA
ccgatccactggggagcaggaattcagctcgtgtagctcattagctccgagct

SEQ ID NO: 111 - DNA, 55 bp, synthetic construct
  misc_feature 1..55: note = polynucleotide
  variation 32..33: note = n is a, c, g, t or u
  source 1..55: mol_type = other DNA
ttcagctcgtgtagctcattagctccgagctnntgggctggtgacccagtcagag

SEQ ID NO: 112 - DNA, 55 bp, synthetic construct
  misc_feature 1..55: note = polynucleotide
  variation 23..24: note = n is a, c, g, t or u
  source 1..55: mol_type = other DNA
atccactggggagcaggaaatannttcagctcgtgtagctcattagctccgagct

SEQ ID NO: 113 - protein, 7 aa, synthetic construct
  REGION 1..7: note = peptide
GSGGSGP

SEQ ID NO: 114 - protein, 223 aa, synthetic construct
  REGION 1..223: note = polypeptide
MPKKKRKVVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGGSGPRPRGTRGKGRRIRR

SEQ ID NO: 115 - DNA, 672 bp, synthetic construct
  misc_feature 1..672: note = polypeptide
  source 1..672: mol_type = other DNA
atgcccaagaagaagagaaaggtggtgaagagcgagctggaggagaagaagagcgagctgagacacaagctgaagtacgtgccccacgagtacatcgagctgatcgagatcgccagaaacagcacccaggacagaatcctggagatgaaggtgatggagttcttcatgaaggtgtacggctacagaggcaagcacctgggcggcagcagaaagcccgacggcgccatctacaccgtgggcagccccatcgactacggcgtgatcgtggacaccaaggcctacagcggcggctacaacctgcccatcggccaggccgacgagatgcagagatacgtggaggagaaccagaccagaaacaagcacatcaaccccaacgagtggtggaaggtgtaccccagcagcgtgaccgagttcaagttcctgttcgtgagcggccacttcaagggcaactacaaggcccagctgaccagactgaaccacatcaccaactgcaacggcgccgtgctgagcgtggaggagctgctgatcggcggcgagatgatcaaggccggcaccctgaccctggaggaggtgagaagaaagttcaacaacggcgagatcaacttcggcagcggcggcagcggccccagacccagaggcaccagaggcaagggcagaagaatcagaagataa

SEQ ID NO: 116 - protein, 7 aa, synthetic construct
  REGION 1..7: note = peptide
GGSGGGP

SEQ ID NO: 117 - DNA, 31 bp, synthetic construct
  misc_feature 1..31: note = polynucleotide
  source 1..31: mol_type = other DNA
ttcagctcgtgtagctcattagctccgagct

SEQ ID NO: 118 - DNA, 31 bp, synthetic construct
  misc_feature 1..31: note = polynucleotide
  source 1..31: mol_type = other DNA
gcaaaacaattatttggttatgccattttgg

SEQ ID NO: 119 - DNA, 53 bp, synthetic construct
  misc_feature 1..53: note = polynucleotide
  source 1..53: mol_type = other DNA
ttcagctcgtgtagctcattagctccgagctcaatgatggatttcgcgccacg

SEQ ID NO: 120 - DNA, 53 bp, synthetic construct
  misc_feature 1..53: note = polynucleotide
  source 1..53: mol_type = other DNA
ttagcttcggttagagcaaagcttcagctcgtgtagctcattagctccgagct

SEQ ID NO: 121 - DNA, 53 bp, synthetic construct
  misc_feature 1..53: note = polynucleotide
  source 1..53: mol_type = other DNA
ttcagctcgtgtagctcattagctccgagctgccaatgatggatttcgcgcca

SEQ ID NO: 122 - DNA, 53 bp, synthetic construct
  misc_feature 1..53: note = polynucleotide
  source 1..53: mol_type = other DNA
agcttcggttagagcaaagcccttcagctcgtgtagctcattagctccgagct

SEQ ID NO: 123 - DNA, 22 bp, synthetic construct
  misc_feature 1..22: note = polynucleotide
  source 1..22: mol_type = other DNA
atgttagaaggtgcaaaatcaa

SEQ ID NO: 124 - DNA, 22 bp, synthetic construct
  misc_feature 1..22: note = polynucleotide
  source 1..22: mol_type = other DNA
ctaacggacttggaatacgaat

SEQ ID NO: 125 - DNA, 2833 bp, synthetic construct
  misc_feature 1..2833: note = polynucleotide
  source 1..2833: mol_type = other DNA
tgctgtaccatacagtctttccgcttaaagaattatattctctctagctacttcacagtgtcctttcctttcgcgtactcctctgtctttttcctatcttctctcttctattaccgagacccgtacatacaaagatctaggtagctcatctccttctaagaagtcagatctccttcctatattctataaagaataagagcccttcttaacttcactctttaattgtaaagagaggcctcatttaggagcgctgttttcatccaactatcaatctcgtaagagaataaagcgtggaaaagaaaggttttgattcgctttataaagaagaaagctttttccggttctaacaggctaattaaattagaaacgcgtctgttcacgtcaggtccgaagggatgtggttgcttttccttttttgcgtcgagattgggttggtgttcagtgtaccgcttgtctagcctatgctttgcatgaacatctcaatgtccaagataaaaagaacgaggggaagaatcgacgaggccagtgttctcgaagagaaaatcgtgatggaaaaagcgtgaggagaattcgaaagtcgatatgttagaaggtgcaaaatcaatgggtgcaggagctgctacaatcgcttcagcgggagctgctatcggtattggaaacgtccttagttcctcgattcattccgtgttaggtatacaaataacagacttacatcacgatgtctttttcttcgttattctgattttggttttcgtatcatggatcttgggtcgcgctttatggcatttccactataaaaaaaatccaatcccgcaaaggattgttcatggaactactatcgagattcttcggaccttatttcttagtatcatccctatgttcattgctataccatcatttgccctgtatgggtattcggactataacagttccgatgaacagtcactcacttttgacagttatacgattccagaagatcatccagaattgggtcaatcacgtttattagaagtcgacaatagagtggttgtaccagcaaacagttctctccgttttattgtaacatctgcggctgtaccttccttaggtgtcaaaggtgatgctgtgccttccttaggtgtcaaaggtgatgctgtgccttccttaggtgtcaaaggtgatgctgtgcctgggcctgggcgggtttttcagacttggacccgagcttttgagcgtttgggcctgttgacggttgcccattgcgccggcaccggaacatcaagctcgggctcggtagtcagtcttccacaggacgaaatatgggccgcccttgagggcgatccccaggcccttccggaagacggccaatttcacgccgtcgcccctgaggggaatccccaggcccttccggaagacgggcaatttcacgccgtcccccctgaggggaatccccaggcccttccggaagacgggcaatttcaggccgtcgcccctgaggggaatccccaggcccttccggaagacgggcaatttcacgccatcgcctttgaccctcttatagcaacacggcaagacgcgtggaatacgctacttgtcttgttgcggcgcagcaccaaaattggcctaaggccaattttgttactaaagcaggggaagatcttggtatagaggaggtcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcaggagggaagctaatgcatcgtttgccccaatgatggcctttttgatctcattcgtattccaagtccgttagtcatcgtttacggtgggtgggtaagcaggaggggatccctgtggttagattagagtaactggccgagaaggttagcgaggttccagctatggtgaagtgaaagatctttcacttcactatagtgggaaagaagacaggtgggagcgagcggagcgagagcaaagcaagctctagtggtgggttgtcttcgcggtcccatttcatcga

SEQ ID NO: 126 - DNA, 31 bp, synthetic construct
  misc_feature 1..31: note = polynucleotide
  source 1..31: mol_type = other DNA
agacttacatcacgatgtctttttcttcgtt

SEQ ID NO: 127 - DNA, 53 bp, synthetic construct
  misc_feature 1..53: note = polynucleotide
  source 1..53: mol_type = other DNA
ttcagctcgtgtagctcattagctccgagctgttatttgtatacctaacacgg

SEQ ID NO: 128 - DNA, 53 bp, synthetic construct
  misc_feature 1..53: note = polynucleotide
  source 1..53: mol_type = other DNA
atacgaaaaccaaaatcagaatttcagctcgtgtagctcattagctccgagct

SEQ ID NO: 129 - DNA, 53 bp, synthetic construct
  misc_feature 1..53: note = polynucleotide
  source 1..53: mol_type = other DNA
ttcagctcgtgtagctcattagctccgagctctgttatttgtatacctaacac

SEQ ID NO: 130 - DNA, 53 bp, synthetic construct
  misc_feature 1..53: note = polynucleotide
  source 1..53: mol_type = other DNA
acgaaaaccaaaatcagaataattcagctcgtgtagctcattagctccgagct

SEQ ID NO: 131 - DNA, 2852 bp, synthetic construct
  misc_feature 1..2852: note = polynucleotide
  source 1..2852: mol_type = other DNA
tgctgtaccatacagtctttccgcttaaagaattatattctctctagctacttcacagtgtcctttcctttcgcgtactcctctgtctttttcctatcttctctcttctattaccgagacccgtacatacaaagatctaggtagctcatctccttctaagaagtcagatctccttcctatattctataaagaataagagcccttcttaacttcactctttaattgtaaagagaggcctcatttaggagcgctgttttcatccaactatcaatctcgtaagagaataaagcgtggaaaagaaaggttttgattcgctttataaagaagaaagctttttccggttctaacaggctaattaaattagaaacgcgtctgttcacgtcaggtccgaagggatgtggttgcttttccttttttgcgtcgagattgggttggtgttcagtgtaccgcttgtctagcctatgctttgcatgaacatctcaatgtccaagataaaaagaacgaggggaagaatcgacgaggccagtgttctcgaagagaaaatcgtgatggaaaaagcgtgaggagaattcgaaagtcgatatgttagaaggtgcaaaatcaatgggtgcaggagctgctacaaatgcttcagcgggagctgctatcggtattggaaacgtccttagttcctcgattcattccgtggcgcgaaatccatcattggcaaaacaattatttggttatgccattttgggctttgctctaaccgaagctaatgcatcgtttgccccaatgatggcctttttgatctcattcgtattccaagtccgttagaggaggtcgactttaaaagcgaacgaaaaacaattgcaaaagcagattgatttttggcaacgtgaatttaggttttggaagtaaaataagttttatttgataaaaattgctaattcagtataattaatatttacgaggtgacataacgtatgaaaaaatcagaggattattcctcctaaatataaaaatttaaaatttaggaggaagttatatatgacttttaatattattgaattagaaaattgggatagaaaagaatattttgaacactattttaatcagcaaactacttatagcattactaaagaaattgatattactttgtttaaagatatgataaaaaagaaaggatatgaaatttatccctctttaatttatgcaattatggaagttgtaaataaaaataaagtgtttagaacaggaattaatagtgagaataaattaggttattgggataagttaaatcctttgtatacagtttttaataagcaaactgaaaaatttactaacatttggactgaatctgataaaaacttcatttctttttataataattataaaaatgacttgcttgaatataaagataaagaagaaatgtttcctaaaaaaccgatacctgaaaacaccataccgatttcaatgattccttggattgattttagttcatttaatttaaatattggtaacaatagcagctttttattgcctattattacgataggtaaattttatagtgagaataataaaatttatataccagttgctctgcaacttcatcattctgtatgtgatggttaccatgcttcactatttatgaatgaatttcaagatataattcatagggtagatgattggatttagtttttagattttgaaagtgaatttaattttatacacgtaagtgatcaggaggaaaaaatccaatcccgcaaaggattgttcatggaactactatcgagattcttcggaccttatttcttagtatcatccctatgttcattgctataccatcatttgccctgtatgggtattcggactataacagttccgatgaacagtcactcacttttgacagttatacgattccagaagatcatccagaattgggtcaatcacgtttattagaagtcgacaatagagtggttgtaccagcaaacagttctctccgttttattgtaacatctgcggctgtaccttccttaggtgtcaaaggtgatgctgtgccttccttaggtgtcaaaggtgatgctgtgccttccttaggtgtcaaaggtgatgctgtgcctgggcctgggcgggtttttcagacttggacccgagcttttgagcgtttgggcctgttgacggttgcccattgcgccggcaccggaacatcaagctcgggctcggtagtcagtcttccacaggacgaaatatgggccgcccttgagggcgatccccaggcccttccggaagacggccaatttcacgccgtcgcccctgaggggaatccccaggcccttccggaagacgggcaatttcacgccgtcccccctgaggggaatccccaggcccttccggaagacgggcaatttcaggccgtcgcccctgaggggaatccccaggcccttccggaagacgggcaatttcacgccatcgcctttgaccctcttatagcaacacggcaagacgcgtggaatacgctacttgtcttgttgcggcgcagcaccaaaattggcctaaggccaattttgttactaaagcaggggaagatcttggtataggaagctaatgcatcgtttgccccaatgatggcctttttgatctcattcgtattccaagtccgttagtcatcgtttacggtgggtgggtaagcaggaggggatccctgtggttagattagagtaactggccgagaaggttagcgaggttccagctatggtgaagtgaaagatctttcacttcactatagtgggaaagaagacaggtgggagcgagcggagcgagagcaaagcaagctctagtggtgggttgtcttcgcggtcccatttcatcga

SEQ ID NO: 132 - protein, 399 aa, synthetic construct
  REGION 1..399: note = polypeptide
MDKAELIPEPPKKKRKVELGTAANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKEGSLEGGSGGDVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKSSADDAKKDAAKKDDAKKDDAKKDGGVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVS

SEQ ID NO: 133 - DNA, 21 bp, synthetic construct
  misc_feature 1..21: note = polynucleotide
  source 1..21: mol_type = other DNA
gggcatctggaccctcctacc

SEQ ID NO: 134 - DNA, 28 bp, synthetic construct
  misc_feature 1..28: note = polynucleotide
  variation 23..28: note = n is a, c, g, t or u
  source 1..28: mol_type = other DNA
aggattgctcaacaaccatgctnnnnnn

SEQ ID NO: 135 - DNA, 28 bp, synthetic construct
  misc_feature 1..28: note = polynucleotide
  variation 1..6: note = n is a, c, g, t or u
  source 1..28: mol_type = other DNA
nnnnnntctggtgagccctctcctgccc

SEQ ID NO: 136 - DNA, 24 bp, synthetic construct
  misc_feature 1..24: note = polynucleotide
  variation 23..24: note = n is a, c, g, t or u
  source 1..24: mol_type = other DNA
ggaggattgctcaacaaccatgnn

SEQ ID NO: 137 - DNA, 24 bp, synthetic construct
  misc_feature 1..24: note = polynucleotide
  variation 1..2: note = n is a, c, g, t or u
  source 1..24: mol_type = other DNA
nnctggtgagccctctcctgcccg

SEQ ID NO: 138 - DNA, 21 bp, synthetic construct
  misc_feature 1..21: note = polynucleotide
  source 1..21: mol_type = other DNA
tacgtctgttgctagattatc

SEQ ID NO: 139 - DNA, 28 bp, synthetic construct
  misc_feature 1..28: note = polynucleotide
  variation 23..28: note = n is a, c, g, t or u
  source 1..28: mol_type = other DNA
atgcttttattttacaggttctnnnnnn

SEQ ID NO: 140 - DNA, 28 bp, synthetic construct
  misc_feature 1..28: note = polynucleotide
  variation 1..6: note = n is a, c, g, t or u
  source 1..28: mol_type = other DNA
nnnnnngtccaaaagtgttaatgcccaa

SEQ ID NO: 141 - DNA, 24 bp, synthetic construct
  misc_feature 1..24: note = polynucleotide
  variation 23..24: note = n is a, c, g, t or u
  source 1..24: mol_type = other DNA
tcatgcttttattttacaggttnn

SEQ ID NO: 142 - DNA, 24 bp, synthetic construct
  misc_feature 1..24: note = polynucleotide
  variation 1..2: note = n is a, c, g, t or u
  source 1..24: mol_type = other DNA
nntccaaaagtgttaatgcccaag

SEQ ID NO: 143 - DNA, 21 bp, synthetic construct
  misc_feature 1..21: note = polynucleotide
  source 1..21: mol_type = other DNA
cagttgagactcagaacttgg

SEQ ID NO: 144 - DNA, 29 bp, synthetic construct
  misc_feature 1..29: note = polynucleotide
  variation 24..29: note = n is a, c, g, t or u
  source 1..29: mol_type = other DNA
ggaattgaggaagactgttactannnnnn

SEQ ID NO: 145 - DNA, 34 bp, synthetic construct
  misc_feature 1..34: note = polynucleotide
  variation 1..6: note = n is a, c, g, t or u
  source 1..34: mol_type = other DNA
nnnnnnaaggcctgcatcatgatggccaattctc

SEQ ID NO: 146 - DNA, 24 bp, synthetic construct
  misc_feature 1..24: note = polynucleotide
  variation 23..24: note = n is a, c, g, t or u
  source 1..24: mol_type = other DNA
ggaattgaggaagactgttactnn

SEQ ID NO: 147 - DNA, 24 bp, synthetic construct
  misc_feature 1..24: note = polynucleotide
  variation 1..2: note = n is a, c, g, t or u
  source 1..24: mol_type = other DNA
nnggcctgcatcatgatggccaat

SEQ ID NO: 148 - DNA, 18 bp, synthetic construct
  misc_feature 1..18: note = polynucleotide
  source 1..18: mol_type = other DNA
catgcttttattttacag

SEQ ID NO: 149 - DNA, 20 bp, synthetic construct
  misc_feature 1..20: note = polynucleotide
  source 1..20: mol_type = other DNA
ctgtgactttcactgtaatc

SEQ ID NO: 150 - DNA, 21 bp, synthetic construct
  misc_feature 1..21: note = polynucleotide
  source 1..21: mol_type = other DNA
caattgtgaattcacatagaa

SEQ ID NO: 151 - DNA, 28 bp, synthetic construct
  misc_feature 1..28: note = polynucleotide
  variation 23..28: note = n is a, c, g, t or u
  source 1..28: mol_type = other DNA
ggtgtcatattatacaatatttnnnnnn

SEQ ID NO: 152 - DNA, 28 bp, synthetic construct
  misc_feature 1..28: note = polynucleotide
  variation 1..6: note = n is a, c, g, t or u
  source 1..28: mol_type = other DNA
nnnnnnaacattaaattataatgtttga

SEQ ID NO: 153 - DNA, 24 bp, synthetic construct
  misc_feature 1..24: note = polynucleotide
  variation 23..24: note = n is a, c, g, t or u
  source 1..24: mol_type = other DNA
ttggtgtcatattatacaatatnn

SEQ ID NO: 154 - DNA, 24 bp, synthetic construct
  misc_feature 1..24: note = polynucleotide
  variation 1..2: note = n is a, c, g, t or u
  source 1..24: mol_type = other DNA
nnacattaaattataatgtttgac

SEQ ID NO: 155 - DNA, 22 bp, synthetic construct
  misc_feature 1..22: note = polynucleotide
  source 1..22: mol_type = other DNA
ctttgtttataactctgagaag

SEQ ID NO: 156 - DNA, 22 bp, synthetic construct
  misc_feature 1..22: note = polynucleotide
  source 1..22: mol_type = other DNA
tcaaaatgcttttgatgcctga

SEQ ID NO: 157 - DNA, 1200 bp, synthetic construct
  misc_feature 1..1200: note = polynucleotide
  source 1..1200: mol_type = other DNA
atggacaaggcagagttgatccctgaaccacctaagaaaaagagaaaggtggagctcggaacagcagctaacttcaaccagtctggaaacattgcagactcttctttgtcttttactttcacaaattcttctaatggaccaaacttgattacaacacagacaaactcacaggctttgtctcagccaatcgcttcttctaatgtgcacgacaacttcatgaacaacgagattacagcttcaaagatcgatgacggtaacaattctaagcctctttctcctggatggactgaccagactgcatacaacgcattcggaatcacaacaggtatgttcaacacaactactatggatgatgtgtacaattacctttttgatgacgaggacacaccaccaaacccaaagaaagagggatcacttgagggtggatccggaggagacgtggtgatgacacagacaccattgtctttgcctgtgtccctaggagaccaggcatcaatctcatgcaggtcatctcagtctcttgtgcactctaatggtaacacttatttgaggtggtacttgcagaagccaggtcagtctccaaaggtgcttatctataaggtttctaacagattctcaggagtgccagataggttctcaggatcaggatctggaacagactttacattgaagatttcaagggttgaggcagaggaccttggagtgtacttctgctctcagtcaacacacgttccatggactttcggtggaggaacaaaacttgagattaaatctagcgctgacgacgctaagaaggacgctgctaagaaggatgacgctaaaaaggacgacgcaaagaaggacggtggtgtgaagcttgacgagactggaggtggtttggtgcagcctggaaggcctatgaaactttcatgcgtggcttctggattcacattctctgactactggatgaactgggttaggcagtcacctgagaagggtttggaatgggtggctcagatcaggaacaagccttacaactatgagacttactactctgattctgtgaaaggaaggttcactatctcaagggacgattcaaagtcttctgtttaccttcagatgaataacttacgcgtagaagacatgggtatctattactgtactggatcttactacggtatggactattggggacagggtacatcagtgacagtgtcatga

SEQ ID NO: 158 - DNA, 20 bp, synthetic construct
  misc_feature 1..20: note = polynucleotide
  source 1..20: mol_type = other DNA
tagggcgctggcaagtgtag

SEQ ID NO: 159 - DNA, 20 bp, synthetic construct
  misc_feature 1..20: note = polynucleotide
  source 1..20: mol_type = other DNA
cataacaccccttgtattac

SEQ ID NO: 160 - DNA, 1120 bp, synthetic construct
  misc_feature 1..1120: note = polynucleotide
  source 1..1120: mol_type = other DNA
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaacaataaaactgtctgcttacataaaca

SEQ ID NO: 161 - DNA, 21 bp, synthetic construct
  misc_feature 1..21: note = polynucleotide
  source 1..21: mol_type = other DNA
tatgagtattcaacatttccg

SEQ ID NO: 162 - DNA, 28 bp, synthetic construct
  misc_feature 1..28: note = polynucleotide
  variation 23..28: note = n is a, c, g, t or u
  source 1..28: mol_type = other DNA
aataatattgaaaaaggaagagnnnnnn

SEQ ID NO: 163 - DNA, 28 bp, synthetic construct
  misc_feature 1..28: note = polynucleotide
  variation 1..6: note = n is a, c, g, t or u
  source 1..28: mol_type = other DNA
nnnnnntgtcgcccttattccctttttt

SEQ ID NO: 164 - DNA, 28 bp, synthetic construct
  misc_feature 1..28: note = polynucleotide
  variation 1..6: note = n is a, c, g, t or u
  source 1..28: mol_type = other DNA
nnnnnnctcttcctttttcaatattatt

SEQ ID NO: 165 - DNA, 28 bp, synthetic construct
  misc_feature 1..28: note = polynucleotide
  variation 23..28: note = n is a, c, g, t or u
  source 1..28: mol_type = other DNA
aaaaaagggaataagggcgacannnnnn

SEQ ID NO: 166 - DNA, 24 bp, synthetic construct
  misc_feature 1..24: note = polynucleotide
  variation 23..24: note = n is a, c, g, t or u
  source 1..24: mol_type = other DNA
caataatattgaaaaaggaagann

SEQ ID NO: 167 - DNA, 24 bp, synthetic construct
  misc_feature 1..24: note = polynucleotide
  variation 1..2: note = n is a, c, g, t or u
  source 1..24: mol_type = other DNA
nntcgcccttattcccttttttgc

SEQ ID NO: 168 - DNA, 24 bp, synthetic construct
  misc_feature 1..24: note = polynucleotide
  variation 1..2: note = n is a, c, g, t or u
  source 1..24: mol_type = other DNA
nntcttcctttttcaatattattg

SEQ ID NO: 169 - DNA, 24 bp, synthetic construct
  misc_feature 1..24: note = polynucleotide
  variation 23..24: note = n is a, c, g, t or u
  source 1..24: mol_type = other DNA
gcaaaaaagggaataagggcgann

SEQ ID NO: 170 - DNA, 21 bp, synthetic construct
  misc_feature 1..21: note = polynucleotide
  source 1..21: mol_type = other DNA
agcattggtaactgtcagacc

SEQ ID NO: 171 - DNA, 28 bp, synthetic construct
  misc_feature 1..28: note = polynucloeitde
  variation 23..28: note = n is a, c, g, t or u
  source 1..28: mol_type = other DNA
gagataggtgcctcactgattannnnnn

SEQ ID NO: 172 - DNA, 28 bp, synthetic construct
  misc_feature 1..28: note = polynucleotide
  variation 1..6: note = n is a, c, g, t or u
  source 1..28: mol_type = other DNA
nnnnnnaagtttactcatatatacttta

SEQ ID NO: 173 - DNA, 28 bp, synthetic construct
  misc_feature 1..28: note = polynucleotide
  variation 1..6: note = n is a, c, g, t or u
  source 1..28: mol_type = other DNA
nnnnnntaatcagtgaggcacctatctc

SEQ ID NO: 174 - DNA, 28 bp, synthetic construct
  misc_feature 1..28: note = polynucleotide
  variation 23..28: note = n is a, c, g, t or u
  source 1..28: mol_type = other DNA
taaagtatatatgagtaaacttnnnnnn

SEQ ID NO: 175 - DNA, 24 bp, synthetic construct
  misc_feature 1..24: note = polynucleotide
  variation 23..24: note = n is a, c, g, t or u
  source 1..24: mol_type = other DNA
tgagataggtgcctcactgattnn

SEQ ID NO: 176 - DNA, 24 bp, synthetic construct
  misc_feature 1..24: note = polynucleotide
  variation 1..2: note = n is a, c, g, t or u
  source 1..24: mol_type = other DNA
nngtttactcatatatactttaga

SEQ ID NO: 177 - DNA, 24 bp, synthetic construct
  misc_feature 1..24: note = polynucloeitde
  variation 1..2: note = n is a, c, g, t or u
  source 1..24: mol_type = other DNA
nnaatcagtgaggcacctatctca

SEQ ID NO: 178 - DNA, 24 bp, synthetic construct
  misc_feature 1..24: note = polynucleotide
  variation 23..24: note = n is a, c, g, t or u
  source 1..24: mol_type = other DNA
tctaaagtatatatgagtaaacnn

SEQ ID NO: 179 - DNA, 1902 bp, synthetic construct
  misc_feature 1..1902: note = polynucleotide
  source 1..1902: mol_type = other DNA
gagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgaattaattctcatgtttgacagcttatcatcgattagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttacatgcaccattccttgcggcggcggtgctcaacggcctcaacctactactgggctgcttcctaatgcaggagtcgcataagggagagcgccgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttgtatcggggaagaacagtatgtcgagctattttttgacttactggggatcaagcctgattgggagaaaataaaatattatattttactggatgaattgttttagtacctagatgtggcgcaacgatgccggcgaca

SEQ ID NO: 180 - DNA, 204 bp, synthetic construct
  misc_feature 1..204: note = polynucleotide
  source 1..204: mol_type = other DNA
ggactgtagaggttccgggtgacagccctccgacgggtgacagccctccgacgggtgacagccctccgaattctagaggatccgggtgacagccctccgacgggtgacagccctccgacgggtgacagccctccgaattcgagctcggtacccggggatctgtcgacctcgatcgagatcttcgcaagacccttcctctatata

SEQ ID NO: 181 - DNA, 1875 bp, synthetic construct; identical to SEQ ID NO: 15
  misc_feature 1..1875: note = polynucleotide
  source 1..1875: mol_type = other DNA

SEQ ID NO: 182 - DNA, 22 bp, synthetic construct
  misc_feature 1..22: note = polynucleotide
  source 1..22: mol_type = other DNA
atctcaagtctctaggactggt

SEQ ID NO: 183 - DNA, 22 bp, synthetic construct
  misc_feature 1..22: note = polynucleotide
  source 1..22: mol_type = other DNA
atctgtgagcaaaggcgaggag

SEQ ID NO: 184 - DNA, 68 bp, synthetic construct
  misc_feature 1..68: note = polynucleotide
  source 1..68: mol_type = other DNA
ccatgggatctcaagtctctaggactggtcttcaaaatctttctcactagtttctacgatcttggcca

SEQ ID NO: 185 - DNA, 62 bp, synthetic construct
  misc_feature 1..62: note = polynucleotide
  source 1..62: mol_type = other DNA
ccatgggatctcaagtctctaggactggtcaaaatctttctcactagtttctacgctggcca

SEQ ID NO: 186 - DNA, 56 bp, synthetic construct
  misc_feature 1..56: note = polynucleotide
  source 1..56: mol_type = other DNA
ccatgggatctcaagtctctaggactggtaatctttctcactagttacgctggcca

SEQ ID NO: 187 - DNA, 53 bp, synthetic construct
  misc_feature 1..53: note = polynucleotide
  source 1..53: mol_type = other DNA
ccatgggatctcaagtctctaggactggtaatctttcttgatcagtctggcca

SEQ ID NO: 188 - DNA, 52 bp, synthetic construct
  misc_feature 1..52: note = polynucleotide
  source 1..52: mol_type = other DNA
ccatgggatctcaagtctctaggactggtaatctttcttgatcagctggcca

SEQ ID NO: 189 - DNA, 51 bp, synthetic construct
  misc_feature 1..51: note = polynucleotide
  source 1..51: mol_type = other DNA
ccatgggatctcaagtctctaggactggtaatctttcttgatcactggcca

SEQ ID NO: 190 - DNA, 50 bp, synthetic construct
  misc_feature 1..50: note = polynucleotide
  source 1..50: mol_type = other DNA
ccatgggatctcaagtctctaggactggtctttctcactagttctggcca

SEQ ID NO: 191 - DNA, 44 bp, synthetic construct
  misc_feature 1..44: note = polynucleotide
  source 1..44: mol_type = other DNA
ccatgggatctcaagtctctaggactggtcttcactagtggcca

SEQ ID NO: 192 - DNA, 37 bp, synthetic construct
  misc_feature 1..37: note = polynucleotide
  source 1..37: mol_type = other DNA
aggaagttcatttcatttggrgaggacacgctgaacc

SEQ ID NO: 193 - DNA, 720 bp, synthetic construct
  misc_feature 1..720: note = polynucleotide
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 194 - DNA, 1809 bp, synthetic construct
  misc_feature 1..1809: note = polynucleotide
  source 1..1809: mol_type = other DNA
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 195 - DNA, 211 bp, synthetic construct
  misc_feature 1..211: note = polynucleotide
  source 1..211: mol_type = other DNA
gtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgac

SEQ ID NO: 196 - DNA, 24 bp, synthetic construct
  misc_feature 1..24: note = polynucleotide
  variation 19..24: note = n is a, c, g, t or u
  source 1..24: mol_type = other DNA
cgggtgacagccctccgannnnnn

SEQ ID NO: 197 - DNA, 24 bp, synthetic construct
  misc_feature 1..24: note = polynucleotide
  variation 1..6: note = n is a, c, g, t or u
  source 1..24: mol_type = other DNA
nnnnnntcggagggctgtcacccg

SEQ ID NO: 198 - DNA, 152 bp, synthetic construct
  misc_feature 1..152: note = polynucleotide
  source 1..152: mol_type = other DNA
tcttcgctattacgccagctggcgaaagggggatgtgctgcaaggcgattaagttgggtaacgccagggttttcccagtcacgacgttgtaaaacgacggccagtgccacccataatacccataatagctgtttgccaaccggttctatata

SEQ ID NO: 199 - DNA, 37 bp, synthetic construct; identical to SEQ ID NO: 192
  misc_feature 1..37: note = polynucleotide
  source 1..37: mol_type = other DNA

SEQ ID NO: 200 - DNA, 720 bp, synthetic construct; identical to SEQ ID NO: 193
  misc_feature 1..720: note = polynucleotide
  source 1..720: mol_type = other DNA

SEQ ID NO: 201 - DNA, 1809 bp, synthetic construct; identical to SEQ ID NO: 194
  misc_feature 1..1809: note = polynucleotide
  source 1..1809: mol_type = other DNA

SEQ ID NO: 202 - DNA, 211 bp, synthetic construct; identical to SEQ ID NO: 195
  misc_feature 1..211: note = polynucloeitde
  source 1..211: mol_type = other DNA

SEQ ID NO: 203 - DNA, 28 bp, synthetic construct
  misc_feature 1..28: note = polynucleotide
  variation 23..28: note = n is a, c, g, t or u
  source 1..28: mol_type = other DNA
gccagggttttcccagtcacgannnnnn

SEQ ID NO: 204 - DNA, 28 bp, synthetic construct
  misc_feature 1..28: note = polynucleotide
  variation 1..6: note = n is a, c, g, t or u
  source 1..28: mol_type = other DNA
nnnnnntcgtgactgggaaaaccctggc

SEQ ID NO: 205 - protein, 238 aa, synthetic construct
  REGION 1..238: note = polypeptide
MHSRSALLYRFLRPASRCFSSSSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFGSGGSGPRPRGTRGKGRRIRR

SEQ ID NO: 206 - DNA, 717 bp, synthetic construct
  misc_feature 1..717: note = polynucleotide
  source 1..717: mol_type = other DNA
atgcattctagatctgctcttctttatagatttcttagaccagcttctagatgtttttcttcttcttctgttaagtctgaacttgaagaaaagaagtctgaacttagacataagcttaagtatgttccacatgaatatattgaacttattgaaattgctagaaattctactcaagatagaattcttgaaatgaaggttatggaattttttatgaaggtttatggatatagaggaaagcatcttggaggatctagaaagccagatggagctatttatactgttggatctccaattgattatggagttattgttgatactaaggcttattctggaggatataatcttccaattggacaagctgatgaaatgcaaagatatgttgaagaaaatcaaactagaaataagcatattaatccaaatgaatggtggaaggtttatccatcttctgttactgaatttaagtttctttttgtttctggacattttaagggaaattataaggctcaacttactagacttaatcatattactaattgtaatggagctgttctttctgttgaagaacttcttattggaggagaaatgattaaggctggaactcttactcttgaagaagttagaagaaagtttaataatggagaaattaattttggatctggaggatctggaccaagaccaagaggaactagaggaaagggaagaagaattagaagataa

SEQ ID NO: 207 - DNA, 43 bp, synthetic construct
  misc_feature 1..43: note = polynucleotide
  variation 21: note = n is a, c, g, t or u
  source 1..43: mol_type = other DNA
ttcacctctaaccgggtgagntccaagggcgaggagctgttca

SEQ ID NO: 208 - DNA, 43 bp, synthetic construct
  misc_feature 1..43: note = polynucleotide
  variation 23: note = n is a, c, g, t or u
  source 1..43: mol_type = other DNA
accatttacgaacgatagccatnttcacctctaaccgggtgag

SEQ ID NO: 209 - DNA, 43 bp, synthetic construct
  misc_feature 1..43: note = polynucleotide
  variation 21: note = n is a, c, g, t or u
  source 1..43: mol_type = other DNA
ttcacctctaaccgggtgagnatggctatcgttcgtaaatggt

SEQ ID NO: 210 - DNA, 43 bp, synthetic construct
  misc_feature 1..43: note = polynucleotide
  variation 23: note = n is a, c, g, t or u
  source 1..43: mol_type = other DNA
tgaacagctcctcgcccttgganttcacctctaaccgggtgag

SEQ ID NO: 211 - DNA, 24 bp, synthetic construct
  misc_feature 1..24: note = polynucleoitde
  variation 1..2: note = n is a, c, g, t or u
  source 1..24: mol_type = other DNA
nntgtccaagggcgaggagctgtt

SEQ ID NO: 212 - DNA, 24 bp, synthetic construct
  misc_feature 1..24: note = polynucleotide
  variation 23..24: note = n is a, c, g, t or u
  source 1..24: mol_type = other DNA
catttacgaacgatagccatggnn

SEQ ID NO: 213 - DNA, 24 bp, synthetic construct
  misc_feature 1..24: note = polynucleotide
  variation 1..2: note = n is a, c, g, t or u
  source 1..24: mol_type = other DNA
nnccatggctatcgttcgtaaatg

SEQ ID NO: 214 - DNA, 24 bp, synthetic construct
  misc_feature 1..24: note = polynucleotide
  variation 23..24: note = n is a, c, g, t or u
  source 1..24: mol_type = other DNA
aacagctcctcgcccttggacann

SEQ ID NO: 215 - protein, 6 aa, synthetic construct
  REGION 1..6: note = peptide
ACTAGT

SEQ ID NO: 216 - protein, 6 aa, synthetic construct
  REGION 1..6: note = peptide
TGATCA

SEQ ID NO: 217 - DNA, 28 bp, synthetic construct
  misc_feature 1..28: note = polynucleotide
  variation 23..28: note = n is a, c, g, t or u
  source 1..28: mol_type = other DNA
ataccagcaccagcaattacaannnnnn